SEQUENCE LISTING (102 sequences)

SEQ ID NO: 1 - protein, 4 aa, Artificial Sequence
  Description of Artificial Sequence Synthetic peptide
GDIR

SEQ ID NO: 2 - protein, 6 aa, Artificial Sequence
  Description of Artificial Sequence Synthetic peptide
LPSTGG

SEQ ID NO: 3 - protein, 5 aa, Artificial Sequence
  Description of Artificial Sequence Synthetic peptide
LPSTG

SEQ ID NO: 4 - protein, 4 aa, Human immunodeficiency virus
REKR

SEQ ID NO: 5 - protein, 9 aa, Human immunodeficiency virus
RRVVEREKR

SEQ ID NO: 6 - protein, 9 aa, Artificial Sequence
  Description of Artificial Sequence Synthetic peptide
ERVVEREKE

SEQ ID NO: 7 - protein, 4 aa, Artificial Sequence
  Description of Artificial Sequence Synthetic peptide
SEKS

SEQ ID NO: 8 - protein, 43 aa, Artificial Sequence
  Description of Artificial Sequence Synthetic polypeptide
MGSLQPLATLYLLGMLVASVLAAENLWVTVYYGVPVWKEANTT

SEQ ID NO: 9 - protein, 33 aa, Artificial Sequence
  Description of Artificial Sequence Synthetic polypeptide
TRPNNNTRKSIRIGPGQTFYATGDIIGNIRQAH

SEQ ID NO: 10 - protein, 24 aa, Homo sapiens
MPMGSLQPLATLYLLGMLVASVLA

SEQ ID NO: 11 - protein, 6 aa, Artificial Sequence
  Description of Artificial Sequence Synthetic peptide
  MOD_RES (3)..(3): Any amino acid
  MOD_RES (6)..(6): Any amino acid
  See specification as filed for detailed description of substitutions and preferred embodiments
LPXTGX

SEQ ID NO: 12 - protein, 13 aa, Artificial Sequence
  Description of Artificial Sequence Synthetic peptide
EADTTLFCASDAK

SEQ ID NO: 13 - protein, 38 aa, Artificial Sequence
  Description of Artificial Sequence Synthetic polypeptide
AYDTEVHNVWATHACVPTDPNPQEIVLEDVTENFNMWK

SEQ ID NO: 14 - protein, 21 aa, Artificial Sequence
  Description of Artificial Sequence Synthetic peptide
QIINMWQGVGQCMYAPPIEGK

SEQ ID NO: 15 - protein, 24 aa, Artificial Sequence
  Description of Artificial Sequence Synthetic peptide
NNMVEQMHEDIISLWDQSLKPCVK

SEQ ID NO: 16 - protein, 15 aa, Artificial Sequence
  Description of Artificial Sequence Synthetic peptide
LINCDTSACTQACPK

SEQ ID NO: 17 - protein, 14 aa, Artificial Sequence
  Description of Artificial Sequence Synthetic peptide
LTPLCVTLDCTNVK

SEQ ID NO: 18 - protein, 11 aa, Artificial Sequence
  Description of Artificial Sequence Synthetic peptide
DCSFDITTEIR

SEQ ID NO: 19 - protein, 13 aa, Artificial Sequence
  Description of Artificial Sequence Synthetic peptide
CNDKKFDGTGPCK

SEQ ID NO: 20 - protein, 33 aa, Artificial Sequence
  Description of Artificial Sequence Synthetic polypeptide
DVSTVQCTHGIKPVVSTQLLLDGSLAEEEIIIR

SEQ ID NO: 21 - protein, 20 aa, Artificial Sequence
  Description of Artificial Sequence Synthetic peptide
VSFEPIPIHYCAPAGFAILK

SEQ ID NO: 22 - protein, 22 aa, Artificial Sequence
  Description of Artificial Sequence Synthetic peptide
TIIVQLDESVEIDCTRPNDNTR

SEQ ID NO: 23 - protein, 10 aa, Artificial Sequence
  Description of Artificial Sequence Synthetic peptide
QAHCDISGTK

SEQ ID NO: 24 - protein, 22 aa, Artificial Sequence
  Description of Artificial Sequence Synthetic peptide
TIIFKPSSGGDLEITTHSFNCR

SEQ ID NO: 25 - protein, 4 aa, Artificial Sequence
  Description of Artificial Sequence Synthetic peptide
ITCK

SEQ ID NO: 26 - protein, 22 aa, Artificial Sequence
  Description of Artificial Sequence Synthetic peptide
GEFFYCDTSGLFDSTWIGDGTK

SEQ ID NO: 27 - protein, 14 aa, Artificial Sequence
  Description of Artificial Sequence Synthetic peptide
NNDNTDDTITLPCR

SEQ ID NO: 28 - protein, 16 aa, Artificial Sequence
  Description of Artificial Sequence Synthetic peptide
LICCTNVPWDSSWSNR

SEQ ID NO: 29 - protein, 13 aa, Artificial Sequence
  Description of Artificial Sequence Synthetic peptide
DQQLLGIWGCSGK

SEQ ID NO: 30 - DNA, 2007 bp, Artificial Sequence
  Description of Artificial Sequence Synthetic polynucleotide
gtcgacaagcttgccaccatgagggtccggggaatccagcgcaactgccagcacctctggaggtggggcacgctgatcctggggatgctgatgatctgcagcgcggctgagaacctgtgggtgacagtgtactacggcgtgcctgtgtggaaggaggccaacaccaccctgttctgcgcctcggacgccaaggcctacgacacggaggtccacaacgtgtgggctacccacgcctgcgtgcccaccgaccccaatcctcaggagatcgtcctggagaacgtgaccgagaacttcaacatgtggaagaacaacatggtggagcagatgcacgaggacatcatcagcctgtgggaccagagcctgaagccctgcgtgaagctgacccccctgtgcgtgaccctgaactgcacgaacgtgaacgtgaccaacaccacgaacaacacggaggagaagggggagatcaagaactgcagcttcaacatcaccaccgagatccgggacaagaagcagaaggtgtacgccctgttctaccggctggacgtcgtgccgatcgacgacaacaacaacaactccagcaactacaggctgatcaactgcaacaccagcgcgatcacccaggcctgccctaaggtgtcgttcgagcccatccccatccactactgcgcgcctgccggcttcgccatcctgaagtgcaacgacaagaagttcaacggcaccggcccctgcaagaacgtcagcaccgtccagtgcacccacggcatcaagcctgtggtgtccacccagctgctcctgaacggcagcctggccgaggaggagatcatcatcaggagcgagaacatcaccaacaacgccaagacgatcatcgtgcagctgaacgagtcggtggagatcaactgcacccggcccaacaacaacacgcggaagagcatccggatcggccctggacaggcgttctacgccacgggcgacatcatcggcgacatcaggcaggcccactgcaacatctcggggacgaagtggaacaagaccctgcagcaggtcgcgaagaagctgagggagcacttcaacaacaagaccatcatcttcaagccgagcagcggcggagacctggagatcaccacgcactcgttcaactgccggggcgagttcttctactgtaacacgtcgggcctgttcaacagcacctggatcggcaacggcacgaagaacaacaacaacactaacgacaccatcaccctgccctgccggatcaagcagatcatcaacatgtggcagggcgtgggccaggctatgtacgcccctcccatcgagggcaagatcacgtgcaagagcaacatcaccggcctgctgctgaccagggacggcgggaacaacaacacgaacgagaccgagatcttcagacctggcggcggagacatgagagacaactggcggagcgagctgtacaagtacaaggtcgtgaagatcgagcccctgggcgtcgcacccaccaagtgcaaggagagggtggtgggcaggcgacgccgtaggcgggcggtcggcatcggcgccgtgttcctgggcttcctgggagcagccggcagcaccatgggagccgcctcgatcaccctgaccgtgcaggcgaggcagctgctgtccggcatcgtgcagcagcagtcgaacctgctgagggcccccgaggcccagcagcacctgctccagctgaccgtgtggggcatcaagcagctccaggccagggtgctggccgtcgagcgctacctgaaggaccagcagctgctcggcatctggggctgcagcggcaagctgatctgctgcaccaccgtgccctggaacagcagctggagcaacaagagccaggacgagatctgggacaacatgacctggatggagtgggagcgggagatcaacaactacaccgacatcatctacagcctgatcgaggagagccagaaccagcaggagaagaacgagcaggagctgctggcgctggactgatctagaggatcc

SEQ ID NO: 31 - DNA, 2007 bp, Artificial Sequence
  Description of Artificial Sequence Synthetic polynucleotide
gtcgacaagcttgccaccatgagggtccggggaatccagcgcaactgccagcacctctggaggtggggcacgctgatcctggggatgctgatgatctgcagcgcggctgagaacctgtgggtgacagtgtactacggcgtgcctgtgtggaaggaggccaacaccaccctgttctgcgcctcggacgccaaggcctacgacacggaggtccacaacgtgtgggctacccacgcctgcgtgcccaccgaccccaatcctcaggagatcgtcctggagaacgtgaccgagaacttcaacatgtggaagaacaacatggtggagcagatgcacgaggacatcatcagcctgtgggaccagagcctgaagccctgcgtgaagctgacccccctgtgcgtgaccctgaactgcacgaacgtgaacgtgaccaacaccacgaacaacacggaggagaagggggagatcaagaactgcagcttcaacatcaccaccgagatccgggacaagaagcagaaggtgtacgccctgttctaccggctggacgtcgtgccgatcgacgacaacaacaacaactccagcaactacaggctgatcaactgcaacaccagcgcgtgcacccaggcctgccctaaggtgtcgttcgagcccatccccatccactactgcgcgcctgccggcttcgccatcctgaagtgcaacgacaagaagttcaacggcaccggcccctgcaagaacgtcagcaccgtccagtgcacccacggcatcaagcctgtggtgtccacccagctgctcctgaacggcagcctggccgaggaggagatcatcatcaggagcgagaacatcaccaacaacgccaagacgatcatcgtgcagctgaacgagtcggtggagatcaactgcacccggcccaacaacaacacgcggaagagcatccggatcggccctggacaggcgttctacgccacgggcgacatcatcggcgacatcaggcaggcccactgcaacatctcggggacgaagtggaacaagaccctgcagcaggtcgcgaagaagctgagggagcacttcaacaacaagaccatcatcttcaagccgagcagcggcggagacctggagatcaccacgcactcgttcaactgccggggcgagttcttctactgtaacacgtcgggcctgttcaacagcacctggatcggcaacggcacgaagaacaacaacaacactaacgacaccatcaccctgccctgccggatcaagcagatcatcaacatgtggcagggcgtgggccagtgtatgtacgcccctcccatcgagggcaagatcacgtgcaagagcaacatcaccggcctgctgctgaccagggacggcgggaacaacaacacgaacgagaccgagatcttcagacctggcggcggagacatgagagacaactggcggagcgagctgtacaagtacaaggtcgtgaagatcgagcccctgggcgtcgcacccaccaagtgcaaggagagggtggtgggcaggcgacgccgtaggcgggcggtcggcatcggcgccgtgttcctgggcttcctgggagcagccggcagcaccatgggagccgcctcgatcaccctgaccgtgcaggcgaggcagctgctgtccggcatcgtgcagcagcagtcgaacctgctgagggcccccgaggcccagcagcacctgctccagctgaccgtgtggggcatcaagcagctccaggccagggtgctggccgtcgagcgctacctgaaggaccagcagctgctcggcatctggggctgcagcggcaagctgatctgctgcaccaccgtgccctggaacagcagctggagcaacaagagccaggacgagatctgggacaacatgacctggatggagtgggagcgggagatcaacaactacaccgacatcatctacagcctgatcgaggagagccagaaccagcaggagaagaacgagcaggagctgctggcgctggactgatctagaggatcc

SEQ ID NO: 32 - DNA, 2007 bp, Artificial Sequence
  Description of Artificial Sequence Synthetic polynucleotide
gtcgacaagcttgccaccatgagggtccggggaatccagcgcaactgccagcacctctggaggtggggcacgctgatcctggggatgctgatgatctgcagcgcggctgagaacctgtgggtgacagtgtactacggcgtgcctgtgtggaaggaggccaacaccaccctgttctgcgcctcggacgccaaggcctacgacacggaggtccacaacgtgtgggctacccacgcctgcgtgcccaccgaccccaatcctcaggagatcgtcctggagaacgtgaccgagaacttcaacatgtggaagaacaacatggtggagcagatgcacgaggacatcatcagcctgtgggaccagagcctgaagccctgcgtgaagctgacccccctgtgcgtgaccctgaactgcacgaacgtgaacgtgaccaacaccacgaacaacacggaggagaagggggagatcaagaactgcagcttcaacatcaccaccgagatccgggacaagaagcagaaggtgtacgccctgttctaccggctggacgtcgtgccgatcgacgacaacaacaacaactccagcaactacaggctgatcaactgcaacaccagcgcgatcacccaggcctgccctaaggtgtcgttcgagcccatccccatccactactgcgcgcctgccggcttcgccatcctgaagtgcaacgacaagaagttcaacggcaccggcccctgcaagaacgtcagcaccgtccagtgcacccacggcatcaagcctgtggtgtccacccagctgctcctgaacggcagcctggccgaggaggagatcatcatcaggagcgagaacatcaccaacaacgccaagacgatcatcgtgcagctgaacgagtcggtggagatcaactgcacccggcccaacaacaacacgcggaagagcatccggatcggccctggacaggcgttctacgccacgggcgacatcatcggcgacatcaggcaggcccactgcaacatctcggggacgaagtggaacaagaccctgcagcaggtcgcgaagaagctgagggagcacttcaacaacaagaccatcatcttcaagccgagcagcggcggagacctggagatcaccacgcactcgttcaactgccggggcgagttcttctactgtaacacgtcgggcctgttcaacagcacctggatcggcaacggcacgaagaacaacaacaacactaacgacaccatcaccctgccctgccggatcaagcagatcatcaacatgtggcagggcgtgggccaggctatgtacgcccctcccatcgagggcaagatcacgtgcaagagcaacatcaccggcctgctgctgaccagggacggcgggaacaacaacacgaacgagaccgagatcttcagacctggcggcggagacatgagagacaactggcggagcgagctgtacaagtacaaggtcgtgaagatcgagcccctgggcgtcgcacccaccaagtgcaaggagagggtggtgggcaggcgacgccgtaggcgggcggtcggcatcggcgccgtgttcctgggcttcctgggagcagccggcagcaccatgggagccgcctcgatgaccctgaccgtgcaggcgaggcagctgctgtccggcatcgtgcagcagcagtcgaacctgctgagggcccccgaggcccagcagcacctgctccagctgaccgtgtggggcatcaagcagctccaggccagggtgctggccgtcgagcgctacctgaaggaccagcagctgctcggcatctggggctgcagcggcaagctgatctgctgcaccaccgtgccctggaacagcagctggagcaacaagagccaggacgagatctgggacaacatgacctggatggagtgggagcgggagatcaacaactacaccgacatcatctacagcctgatcgaggagagccagaaccagcaggagaagaacgagcaggagctgctggcgctggactgatctagaggatcc

SEQ ID NO: 33 - DNA, 2007 bp, Artificial Sequence
  Description of Artificial Sequence Synthetic polynucleotide
gtcgacaagcttgccaccatgagggtccggggaatccagcgcaactgccagcacctctggaggtggggcacgctgatcctggggatgctgatgatctgcagcgcggctgagaacctgtgggtgacagtgtactacggcgtgcctgtgtggaaggaggccaacaccaccctgttctgcgcctcggacgccaaggcctacgacacgaaggtccacaacgtgtgggctacccacgcctgcgtgcccaccgaccccaatcctcaggagatcgtcctggagaacgtgaccgagaacttcaacatgtggaagaacaacatggtggagcagatgcacgaggacatcatcagcctgtgggaccagagcctgaagccctgcgtgaagctgacccccctgtgcgtgaccctgaactgcacgaacgtgaacgtgaccaacaccacgaacaacacggaggagaagggggagatcaagaactgcagcttcaacatcaccaccgagatccgggacaagaagcagaaggtgtacgccctgttctaccggctggacgtcgtgccgatcgacgacaacaacaacaactccagcaactacaggctgatcaactgcaacaccagcgcgatcacccaggcctgccctaaggtgtcgttcgagcccatccccatccactactgcgcgcctgccggcttcgccatcctgaagtgcaacgacaagaagttcaacggcaccggcccctgcaagaacgtcagcaccgtccagtgcacccacggcatcaagcctgtggtgtccacccagctgctcctgaacggcagcctggccgaggaggagatcatcatcaggagcgagaacatcaccaacaacgccaagacgatcatcgtgcagctgaacgagtcggtggagatcaactgcacccggcccaacaacaacacgcggaagagcatccggatcggccctggacagtggttctacgccacgggcgacatcatcggcgacatcaggcaggcccactgcaacatctcggggacgaagtggaacaagaccctgcagcaggtcgcgaagaagctgagggagcacttcaacaacaagaccatcatcttcaagccgagcagcggcggagacctggagatcaccacgcactcgttcaactgccggggcgagttcttctactgtaacacgtcgggcctgttcaacagcacctggatcggcaacggcacgaagaacaacaacaacactaacgacaccatcaccctgccctgccggatcaagcagatcatcaacatgtggcagggcgtgggccaggctatgtacgcccctcccatcgagggcaagatcacgtgcaagagcaacatcaccggcctgctgctgaccagggacggcgggaacaacaacacgaacgagaccgagatcttcagacctggcggcggagacatgagagacaactggcggagcgagctgtacaagtacaaggtcgtgaagatcgagcccctgggcgtcgcacccaccaagtgcaaggagagggtggtgggcaggcgacgccgtaggcgggcggtcggcatcggcgccgtgttcctgggcttcctgggagcagccggcagcaccatgggagccgcctcgatgaccctgaccgtgcaggcgaggcagctgctgtccggcatcgtgcagcagcagtcgaacctgctgagggcccccgaggcccagcagcacctgctccagctgaccgtgtggggcatcaagcagctccaggccagggtgctggccgtcgagcgctacctgaaggaccagcagctgctcggcatctggggctgcagcggcaagctgatctgctgcaccaccgtgccctggaacagcagctggagcaacaagagccaggacgagatctgggacaacatgacctggatggagtgggagcgggagatcaacaactacaccgacatcatctacagcctgatcgaggagagccagaaccagcaggagaagaacgagcaggagctgctggcgctggactgatctagaggatcc

SEQ ID NO: 34 - DNA, 2007 bp, Artificial Sequence
  Description of Artificial Sequence Synthetic polynucleotide
gtcgacaagcttgccaccatgagggtccggggaatccagcgcaactgccagcacctctggaggtggggcacgctgatcctggggatgctgatgatctgcagcgcggctgagaacctgtgggtgacagtgtactacggcgtgcctgtgtggaaggaggccaacaccaccctgttctgcgcctcggacgccaaggcctacgacacggaggtcagaaacgtgtgggctacccacgcctgcgtgcccaccgaccccaatcctcaggagatcgtcctggagaacgtgaccgagaacttcaacatgtggaagaacaacatggtggagcagatgcacgaggacatcatcagcctgtgggaccagagcctgaagccctgcgtgaagctgacccccctgtgcgtgaccctgaactgcacgaacgtgaacgtgaccaacaccacgaacaacacggaggagaagggggagatcaagaactgcagcttcaacatcaccaccgagatccgggacaagaagcagaaggtgtacgccctgttctaccggctggacgtcgtgccgatcgacgacaacaacaacaactccagcaactacaggctgatcaactgcaacaccagcgcgatcacccaggcctgccctaaggtgtcgttcgagcccatccccatccactactgcgcgcctgccggcttcgccatcctgaagtgcaacgacaagaagttcaacggcaccggcccctgcaagaacgtcagcaccgtccagtgcacccacggcatcaagcctgtggtgtccacccagctgctcctgaacggcagcctggccgaggaggagatcatcatcaggagcgagaacatcaccaacaacgccaagacgatcatcgtgcagctgaacgagtcggtggagatcaactgcacccggcccaacaacaacacgcggaagagcatccggatcggccctggacagtggttctacgccacgggcgacatcatcggcgacatcaggcaggcccactgcaacatctcggggacgaagtggaacaagaccctgcagcaggtcgcgaagaagctgagggagcacttcaacaacaagaccatcatcttcaagccgagcagcggcggagacctggagatcaccacgcactcgttcaactgccggggcgagttcttctactgtaacacgtcgggcctgttcaacagcacctggatcggcaacggcacgaagaacaacaacaacactaacgacaccatcaccctgccctgccggatcaagcagatcatcaacatgtggcagggcgtgggccaggctatgtacgcccctcccatcgagggcaagatcacgtgcaagagcaacatcaccggcctgctgctgaccagggacggcgggaacaacaacacgaacgagaccgagatcttcagacctggcggcggagacatgagagacaactggcggagcgagctgtacaagtacaaggtcgtgaagatcgagcccctgggcgtcgcacccaccaagtgcaaggagagggtggtgggcaggcgacgccgtaggcgggcggtcggcatcggcgccgtgttcctgggcttcctgggagcagccggcagcaccatgggagccgcctcgatgaccctgaccgtgcaggcgaggcagctgctgtccggcatcgtgcagcagcagtcgaacctgctgagggcccccgaggcccagcagcacctgctccagctgaccgtgtggggcatcaagcagctccaggccagggtgctggccgtcgagcgctacctgaaggaccagcagctgctcggcatctggggctgcagcggcaagctgatctgctgcaccaccgtgccctggaacagcagctggagcaacaagagccaggacgagatctgggacaacatgacctggatggagtgggagcgggagatcaacaactacaccgacatcatctacagcctgatcgaggagagccagaaccagcaggagaagaacgagcaggagctgctggcgctggactgatctagaggatcc

SEQ ID NO: 35 - DNA, 2025 bp, Artificial Sequence
  Description of Artificial Sequence Synthetic polynucleotide
gtcgacgccaccatgggctccctgcagcccctggccaccctgtacctgctgggcatgctggtggcctccgtgctggccgccgagaacctgtgggtgaccgtgtactacggcgtgcccgtgtggaaggaggccaacaccaccctgttctgcgcctccgacgccaaggcctacgacaccgaggtgcacaacgtgtgggccacccacgcctgcgtgcccaccgaccccaacccccaggagatcgtgctggagaacgtgaccgagaacttcaacatgtggaagaacaacatggtggagcagatgcacgaggacatcatctccctgtgggaccagtccctgaagccctgcgtgaagctgacccccctgtgcgtgaccctgaactgcaccaacgtgaacgtgaccaacaccaccaacaacaccgaggagaagggcgagatcaagaactgctccttcaacatcaccaccgagatccgcgacaagaagcagaaggtgtacgccctgttctaccgcctggacgtggtgcccatcgacgacaacaacaacaactcctccaactaccgcctgatcaactgcaacacctccgccatcacccaggcctgccccaaggtgtccttcgagcccatccccatccactactgcgcccccgccggcttcgccatcctgaagtgcaacgacaagaagttcaacggcaccggcccctgcaagaacgtgtccaccgtgcagtgcacccacggcatcaagcccgtggtgtccacccagctgctgctgaacggctccctggccgaggaggagatcatcatccgctccgagaacatcaccaacaacgccaagaccatcatcgtgcagctgaacgagtccgtggagatcaactgcacccgccccaacaacaacacccgcaagtccatccgcatcggccccggccaggccttctacgccaccggcgacatcatcggcgacatccgccaggcccactgcaacatctccggcaccaagtggaacaagaccctgcagcaggtggccaagaagctgcgcgagcacttcaacaacaagaccatcatcttcaagccctcctccggcggcgacctggagatcaccacccactccttcaactgccgcggcgagttcttctactgcaacacctccggcctgttcaactccacctggatcggcaacggcaccaagaacaacaacaacaccaacgacaccatcaccctgccctgccgcatcaagcagatcatcaacatgtggcagggcgtgggccaggccatgtacgccccccccatcgagggcaagatcacctgcaagtccaacatcaccggcctgctgctgacccgcgacggcggcaacaacaacaccaacgagaccgagatcttccgccccggcggcggcgacatgcgcgacaactggcgctccgagctgtacaagtacaaggtggtgaagatcgagcccctgggcgtggcccccacccgctgcaagcgccgcgtggtgggccgccgccgccgccgccgcgccgtgggcatcggcgccgtgttcctgggcttcctgggcgccgccggctccaccatgggcgccgcctccatgaccctgaccgtgcaggcccgcaacctgctgtccggcatcgtgcagcagcagtccaacctgctgcgcgcccccgaggcccagcagcacctgctgaagctgaccgtgtggggcatcaagcagctgcaggcccgcgtgctggccgtggagcgctacctgcgcgaccagcagctgctgggcatctggggctgctccggcaagctgatctgctgcaccaacgtgccctggaactcctcctggtccaaccgcaacctgtccgagatctgggacaacatgacctggctgcagtgggacaaggagatctccaactacacccagatcatctacggcctgctggaggagtcccagaaccagcaggagaagaacgagcaggacctgctggccctggacggctccggcctgaacgacatcttcgaggcccagaagatcgagtggcacgagtagggatcc

SEQ ID NO: 36 - DNA, 2025 bp, Artificial Sequence
  Description of Artificial Sequence Synthetic polynucleotide
gtcgacgccaccatgggctccctgcagcccctggccaccctgtacctgctgggcatgctggtggcctccgtgctggccgccgagaacctgtgggtgaccgtgtactacggcgtgcccgtgtggaaggaggccaacaccaccctgttctgcgcctccgacgccaaggcctacgacaccgaggtgcacaacgtgtgggccacccacgcctgcgtgcccaccgaccccaacccccaggagatcgtgctggagaacgtgaccgagaacttcaacatgtggaagaacaacatggtggagcagatgcacgaggacatcatctccctgtgggaccagtccctgaagccctgcgtgaagctgacccccctgtgcgtgaccctgaactgcaccaacgtgaacgtgaccaacaccaccaacaacaccgaggagaagggcgagatcaagaactgctccttcaacatcaccaccgagatccgcgacaagaagcagaaggtgtacgccctgttctaccgcctggacgtggtgcccatcgacgacaacaacaacaactcctccaactaccgcctgatcaactgcaacacctccgcctgcacccaggcctgccccaaggtgtccttcgagcccatccccatccactactgcgcccccgccggcttcgccatcctgaagtgcaacgacaagaagttcaacggcaccggcccctgcaagaacgtgtccaccgtgcagtgcacccacggcatcaagcccgtggtgtccacccagctgctgctgaacggctccctggccgaggaggagatcatcatccgctccgagaacatcaccaacaacgccaagaccatcatcgtgcagctgaacgagtccgtggagatcaactgcacccgccccaacaacaacacccgcaagtccatccgcatcggccccggccaggccttctacgccaccggcgacatcatcggcgacatccgccaggcccactgcaacatctccggcaccaagtggaacaagaccctgcagcaggtggccaagaagctgcgcgagcacttcaacaacaagaccatcatcttcaagccctcctccggcggcgacctggagatcaccacccactccttcaactgccgcggcgagttcttctactgcaacacctccggcctgttcaactccacctggatcggcaacggcaccaagaacaacaacaacaccaacgacaccatcaccctgccctgccgcatcaagcagatcatcaacatgtggcagggcgtgggccagtgcatgtacgccccccccatcgagggcaagatcacctgcaagtccaacatcaccggcctgctgctgacccgcgacggcggcaacaacaacaccaacgagaccgagatcttccgccccggcggcggcgacatgcgcgacaactggcgctccgagctgtacaagtacaaggtggtgaagatcgagcccctgggcgtggcccccacccgctgcaagcgccgcgtggtgggccgccgccgccgccgccgcgccgtgggcatcggcgccgtgttcctgggcttcctgggcgccgccggctccaccatgggcgccgcctccatgaccctgaccgtgcaggcccgcaacctgctgtccggcatcgtgcagcagcagtccaacctgctgcgcgcccccgaggcccagcagcacctgctgaagctgaccgtgtggggcatcaagcagctgcaggcccgcgtgctggccgtggagcgctacctgcgcgaccagcagctgctgggcatctggggctgctccggcaagctgatctgctgcaccaacgtgccctggaactcctcctggtccaaccgcaacctgtccgagatctgggacaacatgacctggctgcagtgggacaaggagatctccaactacacccagatcatctacggcctgctggaggagtcccagaaccagcaggagaagaacgagcaggacctgctggccctggacggctccggcctgaacgacatcttcgaggcccagaagatcgagtggcacgagtagggatcc

SEQ ID NO: 37 - DNA, 2025 bp, Artificial Sequence
  Description of Artificial Sequence Synthetic polynucleotide
gtcgacgccaccatgggctccctgcagcccctggccaccctgtacctgctgggcatgctggtggcctccgtgctggccgccgagaacctgtgggtgaccgtgtactacggcgtgcccgtgtggaaggaggccaacaccaccctgttctgcgcctccgacgccaaggcctacgacaccaaggtgcacaacgtgtgggccacccacgcctgcgtgcccaccgaccccaacccccaggagatcgtgctggagaacgtgaccgagaacttcaacatgtggaagaacaacatggtggagcagatgcacgaggacatcatctccctgtgggaccagtccctgaagccctgcgtgaagctgacccccctgtgcgtgaccctgaactgcaccaacgtgaacgtgaccaacaccaccaacaacaccgaggagaagggcgagatcaagaactgctccttcaacatcaccaccgagatccgcgacaagaagcagaaggtgtacgccctgttctaccgcctggacgtggtgcccatcgacgacaacaacaacaactcctccaactaccgcctgatcaactgcaacacctccgccatcacccaggcctgccccaaggtgtccttcgagcccatccccatccactactgcgcccccgccggcttcgccatcctgaagtgcaacgacaagaagttcaacggcaccggcccctgcaagaacgtgtccaccgtgcagtgcacccacggcatcaagcccgtggtgtccacccagctgctgctgaacggctccctggccgaggaggagatcatcatccgctccgagaacatcaccaacaacgccaagaccatcatcgtgcagctgaacgagtccgtggagatcaactgcacccgccccaacaacaacacccgcaagtccatccgcatcggccccggccagtggttctacgccaccggcgacatcatcggcgacatccgccaggcccactgcaacatctccggcaccaagtggaacaagaccctgcagcaggtggccaagaagctgcgcgagcacttcaacaacaagaccatcatcttcaagccctcctccggcggcgacctggagatcaccacccactccttcaactgccgcggcgagttcttctactgcaacacctccggcctgttcaactccacctggatcggcaacggcaccaagaacaacaacaacaccaacgacaccatcaccctgccctgccgcatcaagcagatcatcaacatgtggcagggcgtgggccaggccatgtacgccccccccatcgagggcaagatcacctgcaagtccaacatcaccggcctgctgctgacccgcgacggcggcaacaacaacaccaacgagaccgagatcttccgccccggcggcggcgacatgcgcgacaactggcgctccgagctgtacaagtacaaggtggtgaagatcgagcccctgggcgtggcccccacccgctgcaagcgccgcgtggtgggccgccgccgccgccgccgcgccgtgggcatcggcgccgtgttcctgggcttcctgggcgccgccggctccaccatgggcgccgcctccatgaccctgaccgtgcaggcccgcaacctgctgtccggcatcgtgcagcagcagtccaacctgctgcgcgcccccgaggcccagcagcacctgctgaagctgaccgtgtggggcatcaagcagctgcaggcccgcgtgctggccgtggagcgctacctgcgcgaccagcagctgctgggcatctggggctgctccggcaagctgatctgctgcaccaacgtgccctggaactcctcctggtccaaccgcaacctgtccgagatctgggacaacatgacctggctgcagtgggacaaggagatctccaactacacccagatcatctacggcctgctggaggagtcccagaaccagcaggagaagaacgagcaggacctgctggccctggacggctccggcctgaacgacatcttcgaggcccagaagatcgagtggcacgagtagggatcc

SEQ ID NO: 38 - DNA, 2025 bp, Artificial Sequence
  Description of Artificial Sequence Synthetic polynucleotide
gtcgacgccaccatgggctccctgcagcccctggccaccctgtacctgctgggcatgctggtggcctccgtgctggccgccgagaacctgtgggtgaccgtgtactacggcgtgcccgtgtggaaggaggccaacaccaccctgttctgcgcctccgacgccaaggcctacgacaccgaggtgcgcaacgtgtgggccacccacgcctgcgtgcccaccgaccccaacccccaggagatcgtgctggagaacgtgaccgagaacttcaacatgtggaagaacaacatggtggagcagatgcacgaggacatcatctccctgtgggaccagtccctgaagccctgcgtgaagctgacccccctgtgcgtgaccctgaactgcaccaacgtgaacgtgaccaacaccaccaacaacaccgaggagaagggcgagatcaagaactgctccttcaacatcaccaccgagatccgcgacaagaagcagaaggtgtacgccctgttctaccgcctggacgtggtgcccatcgacgacaacaacaacaactcctccaactaccgcctgatcaactgcaacacctccgccatcacccaggcctgccccaaggtgtccttcgagcccatccccatccactactgcgcccccgccggcttcgccatcctgaagtgcaacgacaagaagttcaacggcaccggcccctgcaagaacgtgtccaccgtgcagtgcacccacggcatcaagcccgtggtgtccacccagctgctgctgaacggctccctggccgaggaggagatcatcatccgctccgagaacatcaccaacaacgccaagaccatcatcgtgcagctgaacgagtccgtggagatcaactgcacccgccccaacaacaacacccgcaagtccatccgcatcggccccggccagtggttctacgccaccggcgacatcatcggcgacatccgccaggcccactgcaacatctccggcaccaagtggaacaagaccctgcagcaggtggccaagaagctgcgcgagcacttcaacaacaagaccatcatcttcaagccctcctccggcggcgacctggagatcaccacccactccttcaactgccgcggcgagttcttctactgcaacacctccggcctgttcaactccacctggatcggcaacggcaccaagaacaacaacaacaccaacgacaccatcaccctgccctgccgcatcaagcagatcatcaacatgtggcagggcgtgggccaggccatgtacgccccccccatcgagggcaagatcacctgcaagtccaacatcaccggcctgctgctgacccgcgacggcggcaacaacaacaccaacgagaccgagatcttccgccccggcggcggcgacatgcgcgacaactggcgctccgagctgtacaagtacaaggtggtgaagatcgagcccctgggcgtggcccccacccgctgcaagcgccgcgtggtgggccgccgccgccgccgccgcgccgtgggcatcggcgccgtgttcctgggcttcctgggcgccgccggctccaccatgggcgccgcctccatgaccctgaccgtgcaggcccgcaacctgctgtccggcatcgtgcagcagcagtccaacctgctgcgcgcccccgaggcccagcagcacctgctgaagctgaccgtgtggggcatcaagcagctgcaggcccgcgtgctggccgtggagcgctacctgcgcgaccagcagctgctgggcatctggggctgctccggcaagctgatctgctgcaccaacgtgccctggaactcctcctggtccaaccgcaacctgtccgagatctgggacaacatgacctggctgcagtgggacaaggagatctccaactacacccagatcatctacggcctgctggaggagtcccagaaccagcaggagaagaacgagcaggacctgctggccctggacggctccggcctgaacgacatcttcgaggcccagaagatcgagtggcacgagtagggatcc

SEQ ID NO: 39 - DNA, 2499 bp, Artificial Sequence
  Description of Artificial Sequence Synthetic polynucleotide
gtcaccgtcgtcgacgctagcaccatgggctcgctccagccgctcgcgacgctgtacctcctgggcatgctcgtggcgtccgtgctggcggccgagaacctgtgggtgacggtgtactacggcgtgcccgtgtggaaggaggccaacaccacgctgttctgcgccagcgacgccaaggcctacgacaccgaggtgcacaacgtgtgggcgacccacgcctgcgtgccgacggaccccaacccccaggagatcgtgctggagaacgtgaccgagaacttcaacatgtggaagaacaacatggtggagcagatgcacgaggacatcatctcgctgtgggaccagtccctgaagccgtgcgtgaagctgacgcccctgtgcgtgaccctggactgcaccaacgtgaaggtgacgtccaccacgtccaacacggaggagaagggggagatcaagaactgctccttcaacatcaccaccgagatccgcgacaagaagcagaaggtgtacgcgctgttctaccggctggacgtggtgccgatcgacgacaacaacaacaactccagcaactaccgcctgatcaactgcaacaccagcgcctgcacccaggcctgcccgaaggtgtccttcgagcccatccccatccactactgcgcgccggccggcttcgccatcctgaagtgcaacgacaagaagttcaacggcaccggcccctgcaagaacgtgtccaccgtgcagtgcacccacgggatcaagcccgtggtgtccacgcagctgctgctgaacggctccctggccgaggaggagatcatcatccgctccgagaacatcacgaacaacgccaagaccatcatcgtgcagctgaacgagtccgtggagatcaactgcaccaggcccaacaacaacacccgcaagtccatccggatcggccctggccaggcgttctacgccaccggcgacatcatcggcgacatccgccaggcgcactgcaacatctcgggcacgaagtggaacaagaccctgcagcaggtggcgaagaagctgcgcgagcacttcaacaacaagaccatcatcttcaagcccagctccggcggcgacctggagatcacgacccactccttcaactgccgcggcgagttcttctactgcaacacctccggcctgttcaactcgacgtggatcgggaacggcacgaagaacaacaacaacaccaacgacaccatcaccctgccctgccgcatcaagcagatcatcaacatgtggcagggcgtgggccagtgcatgtacgcgccgcccatcgagggcaagatcacctgcaagtccaacatcaccggcctgctcctgacgcgcgacggcggcaacaacaacaccaacgagaccgagatcttcaggccgggcggcggcgacatgcgcgacaactggcgctcggagctgtacaagtacaaggtggtgaagatcgagcccctgggcgtggcgccgacgcgctgcaagagacgcgtggtgggccgcagacgaaggagacgggccgtgggcatcggcgcggtgttcctgggcttcctgggagcagctggttcgacgatgggcgcagcttccatgaccctgacagtgcaggcacgcaacctgctctccggcatcgtccagcagcagtcgaacctgcttcgagcccccgaggcgcagcagcacctcctcaagctgaccgtgtggggcatcaagcagctgcaggcacgcgtgctagccgtggagcgctacctccgcgaccagcagctgctcggaatctggggctgctcgggcaagctgatctgctgcaccaacgtgccgtggaacagctcctggtccaaccgcaacctctcggagatctgggacaacatgacctggctccagtgggacaaggagatctcgaactacacccagatcatctacggcctgctggaggagtcccagaaccagcaggagaagaacgagcaggacctgctggccctggacggcggaggatctggcgacattatcaagctgctgaacgagcaagtgaacaaagagatgaacagctccaacctgtacatgagcatgagcagctggtgttacacccacagccttgatggcgccggactgttcctgtttgatcacgccgccgaggaatacgagcacgccaagaagctgatcatcttcctgaacgagaacaatgtgcccgtgcagctgaccagcattagcgccccagagcacaagttcgagggcctgacacagatctttcagaaggcctacgaacacgagcagcacatctccgagagcatcaacaacatcgtggaccacgccattaagagcaaggatcacgccaccttcaattttctgcagtggtacgtggccgaacagcacgaggaagaagtgctgttcaaggacatcctggacaagattgagctgatcggcaacgagaaccacggcctgtatctggccgaccagtacgtgaagggaatcgccaagagccggaagtcctgataatctagaggatcc

SEQ ID NO: 40 - DNA, 1986 bp, Artificial Sequence
  Description of Artificial Sequence Synthetic polynucleotide
gtcgacgctagcaccatgggctcgctccagccgctcgcgacgctgtacctcctgggcatgctcgtggcgtccgtgctggcggccgagaacctgtgggtgacggtgtactacggcgtgcccgtgtggaaggaggccaacaccacgctgttctgcgccagcgacgccaaggcctacgacaccgaggtgcacaacgtgtgggcgacccacgcctgcgtgccgacggaccccaacccccaggagatcgtgctggagaacgtgaccgagaacttcaacatgtggaagaacaacatggtggagcagatgcacgaggacatcatctcgctgtgggaccagtccctgaagccgtgcgtgaagctgacgcccctgtgcgtgaccctggactgcaccaacgtgaaggtgacgtccaccacgtccaacacggaggagaagggggagatcaagaactgctccttcaacatcaccaccgagatccgcgacaagaagcagaaggtgtacgcgctgttctaccggctggacgtggtgccgatcgacgacaacaacaacaactccagcaactaccgcctgatcaactgcaacaccagcgcctgcacccaggcctgcccgaaggtgtccttcgagcccatccccatccactactgcgcgccggccggcttcgccatcctgaagtgcaacgacaagaagttcaacggcaccggcccctgcaagaacgtgtccaccgtgcagtgcacccacgggatcaagcccgtggtgtccacgcagctgctgctgaacggctccctggccgaggaggagatcatcatccgctccgagaacatcacgaacaacgccaagaccatcatcgtgcagctgaacgagtccgtggagatcaactgcaccaggcccaacaacaacacccgcaagtccatccggatcggccctggccaggcgttctacgccaccggcgacatcatcggcgacatccgccaggcgcactgcaacatctcgggcacgaagtggaacaagaccctgcagcaggtggcgaagaagctgcgcgagcacttcaacaacaagaccatcatcttcaagcccagctccggcggcgacctggagatcacgacccactccttcaactgccgcggcgagttcttctactgcaacacctccggcctgttcaactcgacgtggatcgggaacggcacgaagaacaacaacaacaccaacgacaccatcaccctgccctgccgcatcaagcagatcatcaacatgtggcagggcgtgggccagtgcatgtacgcgccgcccatcgagggcaagatcacctgcaagtccaacatcaccggcctgctcctgacgcgcgacggcggcaacaacaacaccaacgagaccgagatcttcaggccgggcggcggcgacatgcgcgacaactggcgctcggagctgtacaagtacaaggtggtgaagatcgagcccctgggcgtggcgccgacgcgctgcaagagacgcgtggtgggccgcagacgaaggagacgggccgtgggcatcggcgcggtgttcctgggcttcctgggagcagctggttcgacgatgggcgcagcttccatgaccctgacagtgcaggcacgcaacctgctctccggcatcgtccagcagcagtcgaacctgcttcgagcccccgaggcgcagcagcacctcctcaagctgaccgtgtggggcatcaagcagctgcaggcacgcgtgctagccgtggagcgctacctccgcgaccagcagctgctcggaatctggggctgctcgggcaagctgatctgctgcaccaacgtgccgtggaacagctcctggtccaaccgcaacctctcggagatctgggacaacatgacctggctccagtgggacaaggagatctcgaactacacccagatcatctacggcctgctggaggagtcccagaaccagcaggagaagaacgagcaggacctgctggccctggactgataatctagaggatcc

SEQ ID NO: 41 - DNA, 1986 bp, Artificial Sequence
  Description of Artificial Sequence Synthetic polynucleotide
gtcgacgctagcaccatgggctcgctccagccgctcgcgacgctgtacctcctgggcatgctcgtggcgtccgtgctggcggccgagaacctgtgggtgacggtgtactacggcgtgcccgtgtggaaggaggccaacaccacgctgttctgcgccagcgacgccaaggcctacgacaccgaggtgcacaacgtgtgggcgacccacgcctgcgtgccgacggaccccaacccccaggagatcgtgctggagaacgtgaccgagaacttcaacatgtggaagaacaacatggtggagcagatgcacgaggacatcatctcgctgtgggaccagtccctgaagccgtgcgtgaagctgacgcccctgtgcgtgaccctgaactgcaccaacgtgaacgtgacgtccaccacgtccaacacggaggagaagggggagatcaagaactgctccttcaacatcaccaccgagatccgcgacaagaagcagaaggtgtacgcgctgttctaccggctggacgtggtgccgatcgacgacaacaacaacaactccagcaactaccgcctgatcaactgcaacaccagcgcctgcacccaggcctgcccgaaggtgtccttcgagcccatccccatccactactgcgcgccggccggcttcgccatcctgaagtgcaacgacaagaagttcaacggcaccggcccctgcaagaacgtgtccaccgtgcagtgcacccacgggatcaagcccgtggtgtccacgcagctgctgctgaacggctccctggccgaggaggagatcatcatccgctccgagaacatcacgaacaacgccaagaccatcatcgtgcagctgaacgagtccgtggagatcaactgcaccaggcccaacaacaacacccgcaagtccatccggatcggccctggccaggcgttctacgccaccggcgacatcatcggcgacatccgccaggcgcactgcaacatctcgggcacgaagtggaacaagaccctgcagcaggtggcgaagaagctgcgcgagcacttcaacaacaagaccatcatcttcaagcccagctccggcggcgacctggagatcacgacccactccttcaactgccgcggcgagttcttctactgcaacacctccggcctgttcaactcgacgtggatcgggaacggcacgaagaacaacaacaacaccaacgacaccatcaccctgccctgccgcatcaagcagatcatcaacatgtggcagggcgtgggccagtgcatgtacgcgccgcccatcgagggcaagatcacctgcaagtccaacatcaccggcctgctcctgacgcgcgacggcggcaacaacaacaccaacgagaccgagatcttcaggccgggcggcggcgacatgcgcgacaactggcgctcggagctgtacaagtacaaggtggtgaagatcgagcccctgggcgtggcgccgacgcgctgcaagagacgcgtggtgggccgcagacgaaggagacgggccgtgggcatcggcgcggtgttcctgggcttcctgggagcagctggttcgacgatgggcgcagcttccatgaccctgacagtgcaggcacgcaacctgctctccggcatcgtccagcagcagtcgaacctgcttcgagcccccgaggcgcagcagcacctcctcaagctgaccgtgtggggcatcaagcagctgcaggcacgcgtgctagccgtggagcgctacctccgcgaccagcagctgctcggaatctggggctgctcgggcaagctgatctgctgcaccaacgtgccgtggaacagctcctggtccaaccgcaacctctcggagatctgggacaacatgacctggctccagtgggacaaggagatctcgaactacacccagatcatctacggcctgctggaggagtcccagaaccagcaggagaagaacgagcaggacctgctggccctggactgataatctagaggatcc

SEQ ID NO: 42 - DNA, 1986 bp, Artificial Sequence
  Description of Artificial Sequence Synthetic polynucleotide
gtcgacgctagcaccatgggctcgctccagccgctcgcgacgctgtacctcctgggcatgctcgtggcgtccgtgctggcggccgagaacctgtgggtgacggtgtactacggcgtgcccgtgtggaaggaggccaacaccacgctgttctgcgccagcgacgccaaggcctacgacaccgaggtgcacaacgtgtgggcgacccacgcctgcgtgccgacggaccccaacccccaggagatcgtgctggagaacgtgaccgagaacttcaacatgtggaagaacaacatggtggagcagatgcacgaggacatcatctcgctgtgggaccagtccctgaagccgtgcgtgaagctgacgcccctgtgcgtgaccctgaactgcaccaacgtgaacgtgacgaacaccacgaacaacacggaggagaagggggagatcaagaactgctccttcaacatcaccaccgagatccgcgacaagaagcagaaggtgtacgcgctgttctaccggctggacgtggtgccgatcgacgacaacaacaacaactccagcaactaccgcctgatcaactgcaacaccagcgcctgcacccaggcctgcccgaaggtgtccttcgagcccatccccatccactactgcgcgccggccggcttcgccatcctgaagtgcaacgacaagaagttcaacggcaccggcccctgcaagaacgtgtccaccgtgcagtgcacccacgggatcaagcccgtggtgtccacgcagctgctgctgaacggctccctggccgaggaggagatcatcatccgctccgagaacatcacgaacaacgccaagaccatcatcgtgcagctgaacgagtccgtggagatcaactgcaccaggcccaacaacaacacccgcaagtccatccggatcggccctggccaggcgttctacgccaccggcgacatcatcggcgacatccgccaggcgcactgcaacatctcgggcacgaagtggaacaagaccctgcagcaggtggcgaagaagctgcgcgagcacttcaacaacaagaccatcatcttcaagcccagctccggcggcgacctggagatcacgacccactccttcaactgccgcggcgagttcttctactgcaacacctccggcctgttcaactcgacgtggatcgggaacggcacgaagaacaacaacaacaccaacgacaccatcaccctgccctgccgcatcaagcagatcatcaacatgtggcagggcgtgggccagtgcatgtacgcgccgcccatcgagggcaagatcacctgcaagtccaacatcaccggcctgctcctgacgcgcgacggcggcaacaacaacaccaacgagaccgagatcttcaggccgggcggcggcgacatgcgcgacaactggcgctcggagctgtacaagtacaaggtggtgaagatcgagcccctgggcgtggcgccgacgcgctgcaagagacgcgtggtgggccgcagacgaaggagacgggccgtgggcatcggcgcggtgttcctgggcttcctgggagcagctggttcgacgatgggcgcagcttccatgaccctgacagtgcaggcacgcaacctgctctccggcatcgtccagcagcagtcgaacctgcttcgagcccccgaggcgcagcagcacctcctcaagctgaccgtgtggggcatcaagcagctgcaggcacgcgtgctagccgtggagcgctacctccgcgaccagcagctgctcggaatctggggctgctcgggcaagctgatctgctgcaccaacgtgccgtggaacagctcctggtccaaccgcaacctctcggagatctgggacaacatgacctggctccagtgggacaaggagatctcgaactacacccagatcatctacggcctgctggaggagtcccagaaccagcaggagaagaacgagcaggacctgctggccctggactgataatctagaggatcc

SEQ ID NO: 43 - DNA, 2025 bp, Artificial Sequence
  Description of Artificial Sequence Synthetic polynucleotide
gtcgacgccaccatgggctccctgcagcccctggccaccctgtacctgctgggcatgctggtggcctccgtgctggccgccgagaacctgtgggtgaccgtgtactacggcgtgcccgtgtggaaggaggccaacaccaccctgttctgcgcctccgacgccaaggcctacgacaccgaggtgcacaacgtgtgggccacccacgcctgcgtgcccaccgaccccaacccccaggagatcgtgctggagaacgtgaccgagaacttcaacatgtggaagaacaacatggtggagcagatgcacgaggacatcatctccctgtgggaccagtccctgaagccctgcgtgaagctgacccccctgtgcgtgaccctgaactgcaccaacgtgaacgtgaccaacaccaccaacaacaccgaggagaagggcgagatcaagaactgctccttcaacatcaccaccgagatccgcgacaagaagcagaaggtgtacgccctgttctaccgcctggacgtggtgcccatcgacgacaacaacaacaactcctccaactaccgcctgatcaactgcaacacctccgcctgcacccaggcctgccccaaggtgtccttcgagcccatccccatccactactgcgcccccgccggcttcgccatcctgaagtgcaacgacaagaagttcaacggcaccggcccctgcaagaacgtgtccaccgtgcagtgcacccacggcatcaagcccgtggtgtccacccagctgctgctgaacggctccctggccgaggaggagatcatcatccgctccgagaacatcaccaacaacgccaagaccatcatcgtgcagctgaacgagtccgtggagatcaactgcacccgccccaacgccaacacccgcaagtccatccgcatcggccccggccaggccttctacgccaccggcgacatcatcggcgacatccgccaggcccactgcaacatctccggcaccaagtggaacaagaccctgcagcaggtggccaagaagctgcgcgagcacttcaacaacaagaccatcatcttcaagccctcctccggcggcgacctggagatcaccacccactccttcaactgccgcggcgagttcttctactgcaacacctccggcctgttcaactccacctggatcggcaacggcaccaagaacaacaacaacaccaacgacaccatcaccctgccctgccgcatcaagcagatcatcaacatgtggcagggcgtgggccagtgcatgtacgccccccccatcgagggcaagatcacctgcaagtccaacatcaccggcctgctgctgacccgcgacggcggcaacaacaacaccaacgagaccgagatcttccgccccggcggcggcgacatgcgcgacaactggcgctccgagctgtacaagtacaaggtggtgaagatcgagcccctgggcgtggcccccacccgctgcaagcgccgcgtggtgggccgccgccgccgccgccgcgccgtgggcatcggcgccgtgttcctgggcttcctgggcgccgccggctccaccatgggcgccgcctccatgaccctgaccgtgcaggcccgcaacctgctgtccggcatcgtgcagcagcagtccaacctgctgcgcgcccccgaggcccagcagcacctgctgaagctgaccgtgtggggcatcaagcagctgcaggcccgcgtgctggccgtggagcgctacctgcgcgaccagcagctgctgggcatctggggctgctccggcaagctgatctgctgcaccaacgtgccctggaactcctcctggtccaaccgcaacctgtccgagatctgggacaacatgacctggctgcagtgggacaaggagatctccaactacacccagatcatctacggcctgctggaggagtcccagaaccagcaggagaagaacgagcaggacctgctggccctggacggctccggcctgaacgacatcttcgaggcccagaagatcgagtggcacgagtagggatcc

SEQ ID NO: 44 - DNA, 2025 bp, Artificial Sequence
  Description of Artificial Sequence Synthetic polynucleotide
gtcgacgccaccatgggctccctgcagcccctggccaccctgtacctgctgggcatgctggtggcctccgtgctggccgccgagaacctgtgggtgaccgtgtactacggcgtgcccgtgtggaaggaggccaacaccaccctgttctgcgcctccgacgccaaggcctacgacaccgaggtgcacaacgtgtgggccacccacgcctgcgtgcccaccgaccccaacccccaggagatcgtgctggagaacgtgaccgagaacttcaacatgtggaagaacaacatggtggagcagatgcacgaggacatcatctccctgtgggaccagtccctgaagccctgcgtgaagctgacccccctgtgcgtgaccctgaactgcaccaacgtgaacgtgaccaacaccaccaacaacaccgaggagaagggcgagatcaagaactgctccttcaacatcaccaccgagatccgcgacaagaagcagaaggtgtacgccctgttctaccgcctggacgtggtgcccatcgacgacaacaacaacaactcctccaactaccgcctgatcaactgcaacacctccgcctgcacccaggcctgccccaaggtgtccttcgagcccatccccatccactactgcgcccccgccggcttcgccatcctgaagtgcaacgacaagaagttcaacggcaccggcccctgcaagaacgtgtccaccgtgcagtgcacccacggcatcaagcccgtggtgtccacccagctgctgctgaacggctccctggccgaggaggagatcatcatccgctccgagaacatcaccaacaacgccaagaccatcatcgtgcagctgaacgagtccgtggagatcaactgcacccgccccaacaacaacacccgcaagtccatccgcatcggccccggccaggccttctacgccaccggcgacatcatcggcgacatccgccaggcccactgcgccatctccggcaccaagtggaacaagaccctgcagcaggtggccaagaagctgcgcgagcacttcaacaacaagaccatcatcttcaagccctcctccggcggcgacctggagatcaccacccactccttcaactgccgcggcgagttcttctactgcaacacctccggcctgttcaactccacctggatcggcaacggcaccaagaacaacaacaacaccaacgacaccatcaccctgccctgccgcatcaagcagatcatcaacatgtggcagggcgtgggccagtgcatgtacgccccccccatcgagggcaagatcacctgcaagtccaacatcaccggcctgctgctgacccgcgacggcggcaacaacaacaccaacgagaccgagatcttccgccccggcggcggcgacatgcgcgacaactggcgctccgagctgtacaagtacaaggtggtgaagatcgagcccctgggcgtggcccccacccgctgcaagcgccgcgtggtgggccgccgccgccgccgccgcgccgtgggcatcggcgccgtgttcctgggcttcctgggcgccgccggctccaccatgggcgccgcctccatgaccctgaccgtgcaggcccgcaacctgctgtccggcatcgtgcagcagcagtccaacctgctgcgcgcccccgaggcccagcagcacctgctgaagctgaccgtgtggggcatcaagcagctgcaggcccgcgtgctggccgtggagcgctacctgcgcgaccagcagctgctgggcatctggggctgctccggcaagctgatctgctgcaccaacgtgccctggaactcctcctggtccaaccgcaacctgtccgagatctgggacaacatgacctggctgcagtgggacaaggagatctccaactacacccagatcatctacggcctgctggaggagtcccagaaccagcaggagaagaacgagcaggacctgctggccctggacggctccggcctgaacgacatcttcgaggcccagaagatcgagtggcacgagtagggatcc

SEQ ID NO: 45 - DNA, 2004 bp, Artificial Sequence
  Description of Artificial Sequence Synthetic polynucleotide
gtcgacgctagcaccatgggctcgctccagccgctcgcgacgctgtacctcctgggcatgctcgtggcgtccgtgctggcggccgagaacctgtgggtgacggtgtactacggcgtgcccgtgtggaaggaggccaacaccacgctgttctgcgccagcgacgccaaggcctacgacaccgaggtgcacaacgtgtgggcgacccacgcctgcgtgccgacggaccccaacccccaggagatcgtgctggagaacgtgaccgagaacttcaacatgtggaagaacaacatggtggagcagatgcacgaggacatcatctcgctgtgggaccagtccctgaagccgtgcgtgaagctgacgcccctgtgcgtgaccctggactgcaccaacgtgaaggtgacgtccaccacgtccaacacggaggagaagggggagatcaagaactgctccttcaacatcaccaccgagatccgcgacaagaagcagaaggtgtacgcgctgttctaccggctggacgtggtgccgatcgacgacaacaacaacaactccagcaactaccgcctgatcaactgcaacaccagcgcctgcacccaggcctgcccgaaggtgtccttcgagcccatccccatccactactgcgcgccggccggcttcgccatcctgaagtgcaacgacaagaagttcaacggcaccggcccctgcaagaacgtgtccaccgtgcagtgcacccacgggatcaagcccgtggtgtccacgcagctgctgctgaacggctccctggccgaggaggagatcatcatccgctccgagaacatcacgaacaacgccaagaccatcatcgtgcagctgaacgagtccgtggagatcaactgcaccaggcccaacaacaacacccgcaagtccatccggatcggccctggccaggcgttctacgccaccggcgacatcatcggcgacatccgccaggcgcactgcaacatctcgggcacgaagtggaacaagaccctgcagcaggtggcgaagaagctgcgcgagcacttcaacaacaagaccatcatcttcaagcccagctccggcggcgacctggagatcacgacccactccttcaactgccgcggcgagttcttctactgcaacacctccggcctgttcaactcgacgtggatcgggaacggcacgaagaacaacaacaacaccaacgacaccatcaccctgccctgccgcatcaagcagatcatcaacatgtggcagggcgtgggccagtgcatgtacgcgccgcccatcgagggcaagatcacctgcaagtccaacatcaccggcctgctcctgacgcgcgacggcggcaacaacaacaccaacgagaccgagatcttcaggccgggcggcggcgacatgcgcgacaactggcgctcggagctgtacaagtacaaggtggtgaagatcgagcccctgggcgtggcgccgacgcgctgcaagagacgcgtggtgggccgcagacgaaggagacgggccgtgggcatcggcgcggtgttcctgggcttcctgggagcagctggttcgacgatgggcgcagcttccatgaccctgacagtgcaggcacgcaacctgctctccggcatcgtccagcagcagtcgaacctgcttcgagcccccgaggcgcagcagcacctcctcaagctgaccgtgtggggcatcaagcagctgcaggcacgcgtgctagccgtggagcgctacctccgcgaccagcagctgctcggaatctggggctgctcgggcaagctgatctgctgcaccaacgtgccgtggaacagctcctggtccaaccgcaacctctcggagatctgggacaacatgacctggctccagtgggacaaggagatctcgaactacacccagatcatctacggcctgctggaggagtcccagaaccagcaggagaagaacgagcaggacctgctggccctggacctgcctagcaccggaggatgataatctagaggatcc

SEQ ID NO: 46 - DNA, 2013 bp, Artificial Sequence
  Description of Artificial Sequence Synthetic polynucleotide
atgggctccctgcagcccctggccaccctgtacctgctgggcatgctggtggcctccgtgctggccgccgagaacctgtgggtgaccgtgtactacggcgtgcccgtgtggaaggaggccaacaccaccctgttctgcgcctccgacgccaaggcctacgacaccgaggtgcacaacgtgtgggccacccacgcctgcgtgcccaccgaccccaacccccaggagatcgtgctggagaacgtgaccgagaacttcaacatgtggaagaacaacatggtggagcagatgcacgaggacatcatctccctgtgggaccagtccctgaagccctgcgtgaagctgacccccctgtgcgtgaccctggactgcaccaacgtgaaggtgaccaacaccaccaacaacaccgaggagaagggcgagatcaagaactgctccttcaacatcaccaccgagatccgcgacaagaagcagaaggtgtacgccctgttctaccgcctggacgtggtgcccatcgacgacaacaacaacaactcctccaactaccgcctgatcaactgcaacacctccgcctgcacccaggcctgccccaaggtgtccttcgagcccatccccatccactactgcgcccccgccggcttcgccatcctgaagtgcaacgacaagaagttcaacggcaccggcccctgcaagaacgtgtccaccgtgcagtgcacccacggcatcaagcccgtggtgtccacccagctgctgctgaacggctccctggccgaggaggagatcatcatccgctccgagaacatcaccaacaacgccaagaccatcatcgtgcagctgaacgagtccgtggagatcaactgcacccgccccaacaacaacacccgcaagtccatccgcatcggccccggccaggccttctacgccaccggcgacatcatcggcgacatccgccaggcccactgcaacatctccggcaccaagtggaacaagaccctgcagcaggtggccaagaagctgcgcgagcacttcaacaacaagaccatcatcttcaagccctcctccggcggcgacctggagatcaccacccactccttcaactgccgcggcgagttcttctactgcaacacctccggcctgttcaactccacctggatcggcaacggcaccaagaacaacaacaacaccaacgacaccatcaccctgccctgccgcatcaagcagatcatcaacatgtggcagggcgtgggccagtgcatgtacgccccccccatcgagggcaagatcacctgcaagtccaacatcaccggcctgctgctgacccgcgacggcggcaacaacaacaccaacgagaccgagatcttccgccccggcggcggcgacatgcgcgacaactggcgctccgagctgtacaagtacaaggtggtgaagatcgagcccctgggcgtggcccccacccgctgcaagcgccgcgtggtgggccgccgccgccgccgccgcgccgtgggcatcggcgccgtgttcctgggcttcctgggcgccgccggctccaccatgggcgccgcctccatgaccctgaccgtgcaggcccgcaacctgctgtccggcatcgtgcagcagcagtccaacctgctgcgcgcccccgaggcccagcagcacctgctgaagctgaccgtgtggggcatcaagcagctgcaggcccgcgtgctggccgtggagcgctacctgcgcgaccagcagctgctgggcatctggggctgctccggcaagctgatctgctgcaccaacgtgccctggaactcctcctggtccaaccgcaacctgtccgagatctgggacaacatgacctggctgcagtgggacaaggagatctccaactacacccagatcatctacggcctgctggaggagtcccagaaccagcaggagaagaacgagcaggacctgctggccctggacggctccggcctgaacgacatcttcgaggcccagaagatcgagtggcacgagtagggatcc

SEQ ID NO: 47 - DNA, 2013 bp, Artificial Sequence
  Description of Artificial Sequence Synthetic polynucleotide
atgggctccctgcagcccctggccaccctgtacctgctgggcatgctggtggcctccgtgctggccgccgagaacctgtgggtgaccgtgtactacggcgtgcccgtgtggaaggaggccaacaccaccctgttctgcgcctccgacgccaaggcctacgacaccgaggtgcacaacgtgtgggccacccacgcctgcgtgcccaccgaccccaacccccaggagatcgtgctggagaacgtgaccgagaacttcaacatgtggaagaacaacatggtggagcagatgcacgaggacatcatctccctgtgggaccagtccctgaagccctgcgtgaagctgacccccctgtgcgtgaccctgaactgcaccaacgtgaacgtgaccagcaccaccagcaacaccgaggagaagggcgagatcaagaactgctccttcaacatcaccaccgagatccgcgacaagaagcagaaggtgtacgccctgttctaccgcctggacgtggtgcccatcgacgacaacaacaacaactcctccaactaccgcctgatcaactgcaacacctccgcctgcacccaggcctgccccaaggtgtccttcgagcccatccccatccactactgcgcccccgccggcttcgccatcctgaagtgcaacgacaagaagttcaacggcaccggcccctgcaagaacgtgtccaccgtgcagtgcacccacggcatcaagcccgtggtgtccacccagctgctgctgaacggctccctggccgaggaggagatcatcatccgctccgagaacatcaccaacaacgccaagaccatcatcgtgcagctgaacgagtccgtggagatcaactgcacccgccccaacaacaacacccgcaagtccatccgcatcggccccggccaggccttctacgccaccggcgacatcatcggcgacatccgccaggcccactgcaacatctccggcaccaagtggaacaagaccctgcagcaggtggccaagaagctgcgcgagcacttcaacaacaagaccatcatcttcaagccctcctccggcggcgacctggagatcaccacccactccttcaactgccgcggcgagttcttctactgcaacacctccggcctgttcaactccacctggatcggcaacggcaccaagaacaacaacaacaccaacgacaccatcaccctgccctgccgcatcaagcagatcatcaacatgtggcagggcgtgggccagtgcatgtacgccccccccatcgagggcaagatcacctgcaagtccaacatcaccggcctgctgctgacccgcgacggcggcaacaacaacaccaacgagaccgagatcttccgccccggcggcggcgacatgcgcgacaactggcgctccgagctgtacaagtacaaggtggtgaagatcgagcccctgggcgtggcccccacccgctgcaagcgccgcgtggtgggccgccgccgccgccgccgcgccgtgggcatcggcgccgtgttcctgggcttcctgggcgccgccggctccaccatgggcgccgcctccatgaccctgaccgtgcaggcccgcaacctgctgtccggcatcgtgcagcagcagtccaacctgctgcgcgcccccgaggcccagcagcacctgctgaagctgaccgtgtggggcatcaagcagctgcaggcccgcgtgctggccgtggagcgctacctgcgcgaccagcagctgctgggcatctggggctgctccggcaagctgatctgctgcaccaacgtgccctggaactcctcctggtccaaccgcaacctgtccgagatctgggacaacatgacctggctgcagtgggacaaggagatctccaactacacccagatcatctacggcctgctggaggagtcccagaaccagcaggagaagaacgagcaggacctgctggccctggacggctccggcctgaacgacatcttcgaggcccagaagatcgagtggcacgagtagggatcc

SEQ ID NO: 48 - DNA, 2013 bp, Artificial Sequence
  Description of Artificial Sequence Synthetic polynucleotide
atgggctccctgcagcccctggccaccctgtacctgctgggcatgctggtggcctccgtgctggccgccgagaacctgtgggtgaccgtgtactacggcgtgcccgtgtggaaggaggccaacaccaccctgttctgcgcctccgacgccaaggcctacgacaccgaggtgcacaacgtgtgggccacccacgcctgcgtgcccaccgaccccaacccccaggagatcgtgctggagaacgtgaccgagaacttcaacatgtggaagaacaacatggtggagcagatgcacgaggacatcatctccctgtgggaccagtccctgaagccctgcgtgaagctgacccccctgtgcgtgaccctggactgcaccaacgtgaaggtgaccagcaccaccagcaacaccgaggagaagggcgagatcaagaactgctccttcaacatcaccaccgagatccgcgacaagaagcagaaggtgtacgccctgttctaccgcctggacgtggtgcccatcgacgacaacaacaacaactcctccaactaccgcctgatcaactgcaacacctccgcctgcacccaggcctgccccaaggtgtccttcgagcccatccccatccactactgcgcccccgccggcttcgccatcctgaagtgcaacgacaagaagttcaacggcaccggcccctgcaagaacgtgtccaccgtgcagtgcacccacggcatcaagcccgtggtgtccacccagctgctgctgaacggctccctggccgaggaggagatcatcatccgctccgagaacatcaccaacaacgccaagaccatcatcgtgcagctgaacgagtccgtggagatcaactgcacccgccccaacaacaacacccgcaagtccatccgcatcggccccggccaggccttctacgccaccggcgacatcatcggcgacatccgccaggcccactgcaacatctccggcaccaagtggaacaagaccctgcagcaggtggccaagaagctgcgcgagcacttcaacaacaagaccatcatcttcaagccctcctccggcggcgacctggagatcaccacccactccttcaactgccgcggcgagttcttctactgcaacacctccggcctgttcaactccacctggatcggcaacggcaccaagaacaacaacaacaccaacgacaccatcaccctgccctgccgcatcaagcagatcatcaacatgtggcagggcgtgggccagtgcatgtacgccccccccatcgagggcaagatcacctgcaagtccaacatcaccggcctgctgctgacccgcgacggcggcaacaacaacaccaacgagaccgagatcttccgccccggcggcggcgacatgcgcgacaactggcgctccgagctgtacaagtacaaggtggtgaagatcgagcccctgggcgtggcccccacccgctgcaagcgccgcgtggtgggccgccgccgccgccgccgcgccgtgggcatcggcgccgtgttcctgggcttcctgggcgccgccggctccaccatgggcgccgcctccatgaccctgaccgtgcaggcccgcaacctgctgtccggcatcgtgcagcagcagtccaacctgctgcgcgcccccgaggcccagcagcacctgctgaagctgaccgtgtggggcatcaagcagctgcaggcccgcgtgctggccgtggagcgctacctgcgcgaccagcagctgctgggcatctggggctgctccggcaagctgatctgctgcaccaacgtgccctggaactcctcctggtccaaccgcaacctgtccgagatctgggacaacatgacctggctgcagtgggacaaggagatctccaactacacccagatcatctacggcctgctggaggagtcccagaaccagcaggagaagaacgagcaggacctgctggccctggacggctccggcctgaacgacatcttcgaggcccagaagatcgagtggcacgagtagggatcc

SEQ ID NO: 49 - DNA, 1973 bp, Artificial Sequence
  Description of Artificial Sequence Synthetic polynucleotide
atgggctcgctccagccgctcgcgacgctgtacctcctgggcatgctcgtggcgtccgtgctggcggccgagaacctgtgggtgacggtgtactacggcgtgcccgtgtggaaggaggccaacaccacgctgttctgcgccagcgacgccaaggcctacgacaccaaggtgcacaacgtgtgggcgacccacgcctgcgtgccgacggaccccaacccccaggagatcgtgctggagaacgtgaccgagaacttcaacatgtggaagaacaacatggtggagcagatgcacgaggacatcatctcgctgtgggaccagtccctgaagccgtgcgtgaagctgacgcccctgtgcgtgaccctgaactgcaccaacgtgaacgtgacgaacaccacgaacaacacggaggagaagggggagatcaagaactgctccttcaacatcaccaccgagatccgcgacaagaagcagaaggtgtacgcgctgttctaccggctggacgtggtgccgatcgacgacaacaacaacaactccagcaactaccgcctgatcaactgcaacaccagcgcctgcacccaggcctgcccgaaggtgtccttcgagcccatccccatccactactgcgcgccggccggcttcgccatcctgaagtgcaacgacaagaagttcaacggcaccggcccctgcaagaacgtgtccaccgtgcagtgcacccacgggatcaagcccgtggtgtccacgcagctgctgctgaacggctccctggccgaggaggagatcatcatccgctccgagaacatcacgaacaacgccaagaccatcatcgtgcagctgaacgagtccgtggagatcaactgcaccaggcccaacaacaacacccgcaagtccatccggatcggccctggccagtggttctacgccaccggcgacatcatcggcgacatccgccaggcgcactgcaacatctcgggcacgaagtggaacaagaccctgcagcaggtggcgaagaagctgcgcgagcacttcaacaacaagaccatcatcttcaagcccagctccggcggcgacctggagatcacgacccactccttcaactgccgcggcgagttcttctactgcaacacctccggcctgttcaactcgacgtggatcgggaacggcacgaagaacaacaacaacaccaacgacaccatcaccctgccctgccgcatcaagcagatcatcaacatgtggcagggcgtgggccagtgcatgtacgcgccgcccatcgagggcaagatcacctgcaagtccaacatcaccggcctgctcctgacgcgcgacggcggcaacaacaacaccaacgagaccgagatcttcaggccgggcggcggcgacatgcgcgacaactggcgctcggagctgtacaagtacaaggtggtgaagatcgagcccctgggcgtggcgccgacgcgctgcaagagacgcgtggtgggccgcagacgaaggagacgggccgtgggcatcggcgcggtgttcctgggcttcctgggagcagctggttcgacgatgggcgcagcttccatgaccctgacagtgcaggcacgcaacctgctctccggcatcgtccagcagcagtcgaacctgcttcgagcccccgaggcgcagcagcacctcctcaagctgaccgtgtggggcatcaagcagctgcaggcacgcgtgctagccgtggagcgctacctccgcgaccagcagctgctcggaatctggggctgctcgggcaagctgatctgctgcaccaacgtgccgtggaacagctcctggtccaaccgcaacctctcggagatctgggacaacatgacctggctccagtgggacaaggagatctcgaactacacccagatcatctacggcctgctggaggagtcccagaaccagcaggagaagaacgagcaggacctgctggccctggactgataatctagaggatccag

SEQ ID NO: 50 - DNA, 1905 bp, Artificial Sequence
  Description of Artificial Sequence Synthetic polynucleotide
aagcttgtcgacaccatgcgcgtgcgcggcatccagcgcaactgccagcacctgtggcgctggggcaccctgatcctgggcatgctgatgatctgctccgccgccgagaacctgtgggtgaccgtgtactacggcgtgcccgtgtggaaggaggccaacaccaccctgttctgcgcctccgacgccaaggcctacgacaccgaggtgcacaacgtgtgggccacccacgcctgcgtgcccaccgaccccaacccccaggagatcgtgctggagaacgtgaccgagaacttcaacatgtggaagaacaacatggtggagcagatgcacgaggacatcatctccctgtgggaccagtccctgaagccctgcgtgaagctgacccccctgtgcgtgaccctgaactgcaccaacgtgaacgtgaccgccaccaccaacaacaccgaggagaagggcgagatcaagaactgctccttcaacatcaccaccgagatccgcgacaagaagcagaaggtgtacgccctgttctaccgcctggacgtggtgcccatcgacgacaacaacaacaactcctccaactaccgcctgatcaactgcaacacctccgccatcacccaggcctgccccaaggtgtccttcgagcccatccccatccactactgcgcccccgccggcttcgccatcctgaagtgcaacgacaagaagttcaacggcaccggcccctgcaagaacgtgtccaccgtgcagtgcacccacggcatcaagcccgtggtgtccacccagctgctgctgaacggctccctggccgaggaggagatcatcatccgctccgagaacatcaccaacaacgccaagaccatcatcgtgcagctgaacgagtccgtggagatcaactgcacccgccccaacaacaacacccgcaagtccatccgcatcggccccggccaggccttctacgccaccggcgacatcatcggcgacatccgccaggcccactgcaacatctccggcaccaagtggaacaagaccctgcagcaggtggccaagaagctgcgcgagcacttcaacaacaagaccatcatcttcaagccctcctccggcggcgacctggagatcaccacccactccttcaactgccgcggcgagttcttctactgcaacacctccggcctgttcaactccacctggatcggcaacggcaccaagaacaacaacaacaccaacgacaccatcaccctgccctgccgcatcaagcagatcatcaacatgtggcagggcgtgggccaggccatgtacgccccccccatcgagggcaagatcacctgcaagtccaacatcaccggcctgctgctgacccgcgacggcggcaacaacaacaccaacgagaccgagatcttccgccccggcggcggcgacatgcgcgacaactggcgctccgagctgtacaagtacaaggtggtgaagatcgagcccctgggcgtggcccccaccaaggccaagctgaccgtgcaggcccgccagctgctgtccggcatcgtgcagcagcagtccaacctgctgcgcgccatcgaggcccagcagcacctgctgcagctgaccgtgtggggcatcaagcagctgcaggcccgcgtgctggccgtggagcgctacctgaaggaccagcagctggagatctgggacaacatgacctggatggagtgggagcgcgagatcaacaactacaccgacatcatctactccctgatcgaggagtcccagaaccagcaggagaagaacgagcaggagctgctggccctggacaagtgggcctccctgtggaactggttcgacatcaccaactggctgtggggcctgaacgacatcttcgaggcccagaagatcgagtggcacgagtagggatcctctaga

SEQ ID NO: 51 - DNA, 1905 bp, Artificial Sequence
  Description of Artificial Sequence Synthetic polynucleotide
aagcttgtcgacaccatgcgcgtgcgcggcatccagcgcaactgccagcacctgtggcgctggggcaccctgatcctgggcatgctgatgatctgctccgccgccgagaacctgtgggtgaccgtgtactacggcgtgcccgtgtggaaggaggccaacaccaccctgttctgcgcctccgacgccaaggcctacgacaccgaggtgcacaacgtgtgggccacccacgcctgcgtgcccaccgaccccaacccccaggagatcgtgctggagaacgtgaccgagaacttcaacatgtggaagaacaacatggtggagcagatgcacgaggacatcatctccctgtgggaccagtccctgaagccctgcgtgaagctgacccccctgtgcgtgaccctgaactgcaccaacgtgaacgtgaccaacaccaccgccaacaccgaggagaagggcgagatcaagaactgctccttcaacatcaccaccgagatccgcgacaagaagcagaaggtgtacgccctgttctaccgcctggacgtggtgcccatcgacgacaacaacaacaactcctccaactaccgcctgatcaactgcaacacctccgccatcacccaggcctgccccaaggtgtccttcgagcccatccccatccactactgcgcccccgccggcttcgccatcctgaagtgcaacgacaagaagttcaacggcaccggcccctgcaagaacgtgtccaccgtgcagtgcacccacggcatcaagcccgtggtgtccacccagctgctgctgaacggctccctggccgaggaggagatcatcatccgctccgagaacatcaccaacaacgccaagaccatcatcgtgcagctgaacgagtccgtggagatcaactgcacccgccccaacaacaacacccgcaagtccatccgcatcggccccggccaggccttctacgccaccggcgacatcatcggcgacatccgccaggcccactgcaacatctccggcaccaagtggaacaagaccctgcagcaggtggccaagaagctgcgcgagcacttcaacaacaagaccatcatcttcaagccctcctccggcggcgacctggagatcaccacccactccttcaactgccgcggcgagttcttctactgcaacacctccggcctgttcaactccacctggatcggcaacggcaccaagaacaacaacaacaccaacgacaccatcaccctgccctgccgcatcaagcagatcatcaacatgtggcagggcgtgggccaggccatgtacgccccccccatcgagggcaagatcacctgcaagtccaacatcaccggcctgctgctgacccgcgacggcggcaacaacaacaccaacgagaccgagatcttccgccccggcggcggcgacatgcgcgacaactggcgctccgagctgtacaagtacaaggtggtgaagatcgagcccctgggcgtggcccccaccaaggccaagctgaccgtgcaggcccgccagctgctgtccggcatcgtgcagcagcagtccaacctgctgcgcgccatcgaggcccagcagcacctgctgcagctgaccgtgtggggcatcaagcagctgcaggcccgcgtgctggccgtggagcgctacctgaaggaccagcagctggagatctgggacaacatgacctggatggagtgggagcgcgagatcaacaactacaccgacatcatctactccctgatcgaggagtcccagaaccagcaggagaagaacgagcaggagctgctggccctggacaagtgggcctccctgtggaactggttcgacatcaccaactggctgtggggcctgaacgacatcttcgaggcccagaagatcgagtggcacgagtagggatcctctaga

SEQ ID NO: 52 - DNA, 1905 bp, Artificial Sequence
  Description of Artificial Sequence Synthetic polynucleotide
aagcttgtcgacaccatgcgcgtgcgcggcatccagcgcaactgccagcacctgtggcgctggggcaccctgatcctgggcatgctgatgatctgctccgccgccgagaacctgtgggtgaccgtgtactacggcgtgcccgtgtggaaggaggccaacaccaccctgttctgcgcctccgacgccaaggcctacgacaccgaggtgcacaacgtgtgggccacccacgcctgcgtgcccaccgaccccaacccccaggagatcgtgctggagaacgtgaccgagaacttcaacatgtggaagaacaacatggtggagcagatgcacgaggacatcatctccctgtgggaccagtccctgaagccctgcgtgaagctgacccccctgtgcgtgaccctgcaatgcaccaacgtgcaagtgacccaaaccacccaaaacaccgaggagaagggcgagatcaagaactgctccttcaacatcaccaccgagatccgcgacaagaagcagaaggtgtacgccctgttctaccgcctggacgtggtgcccatcgacgacaacaacaacaactcctccaactaccgcctgatcaactgcaacacctccgccatcacccaggcctgccccaaggtgtccttcgagcccatccccatccactactgcgcccccgccggcttcgccatcctgaagtgcaacgacaagaagttcaacggcaccggcccctgcaagaacgtgtccaccgtgcagtgcacccacggcatcaagcccgtggtgtccacccagctgctgctgaacggctccctggccgaggaggagatcatcatccgctccgagaacatcaccaacaacgccaagaccatcatcgtgcagctgaacgagtccgtggagatcaactgcacccgccccaacaacaacacccgcaagtccatccgcatcggccccggccaggccttctacgccaccggcgacatcatcggcgacatccgccaggcccactgcaacatctccggcaccaagtggaacaagaccctgcagcaggtggccaagaagctgcgcgagcacttcaacaacaagaccatcatcttcaagccctcctccggcggcgacctggagatcaccacccactccttcaactgccgcggcgagttcttctactgcaacacctccggcctgttcaactccacctggatcggcaacggcaccaagaacaacaacaacaccaacgacaccatcaccctgccctgccgcatcaagcagatcatcaacatgtggcagggcgtgggccaggccatgtacgccccccccatcgagggcaagatcacctgcaagtccaacatcaccggcctgctgctgacccgcgacggcggcaacaacaacaccaacgagaccgagatcttccgccccggcggcggcgacatgcgcgacaactggcgctccgagctgtacaagtacaaggtggtgaagatcgagcccctgggcgtggcccccaccaaggccaagctgaccgtgcaggcccgccagctgctgtccggcatcgtgcagcagcagtccaacctgctgcgcgccatcgaggcccagcagcacctgctgcagctgaccgtgtggggcatcaagcagctgcaggcccgcgtgctggccgtggagcgctacctgaaggaccagcagctggagatctgggacaacatgacctggatggagtgggagcgcgagatcaacaactacaccgacatcatctactccctgatcgaggagtcccagaaccagcaggagaagaacgagcaggagctgctggccctggacaagtgggcctccctgtggaactggttcgacatcaccaactggctgtggggcctgaacgacatcttcgaggcccagaagatcgagtggcacgagtagggatcctctaga

SEQ ID NO: 53 - protein, 658 aa, Artificial Sequence
  Description of Artificial Sequence Synthetic polypeptide
MRVRGIQRNCQHLWRWGTLILGMLMICSAAENLWVTVYYGVPVWKEANTTLFCASDAKAYDTEVHNVWATHACVPTDPNPQEIVLENVTENFNMWKNNMVEQMHEDIISLWDQSLKPCVKLTPLCVTLNCTNVNVTNTTNNTEEKGEIKNCSFNITTEIRDKKQKVYALFYRLDVVPIDDNNNNSSNYRLINCNTSAITQACPKVSFEPIPIHYCAPAGFAILKCNDKKFNGTGPCKNVSTVQCTHGIKPVVSTQLLLNGSLAEEEIIIRSENITNNAKTIIVQLNESVEINCTRPNNNTRKSIRIGPGQAFYATGDIIGDIRQAHCNISGTKWNKTLQQVAKKLREHFNNKTIIFKPSSGGDLEITTHSFNCRGEFFYCNTSGLFNSTWIGNGTKNNNNTNDTITLPCRIKQIINMWQGVGQAMYAPPIEGKITCKSNITGLLLTRDGGNNNTNETEIFRPGGGDMRDNWRSELYKYKVVKIEPLGVAPTKCKERVVGRRRRRRAVGIGAVFLGFLGAAGSTMGAASITLTVQARQLLSGIVQQQSNLLRAPEAQQHLLQLTVWGIKQLQARVLAVERYLKDQQLLGIWGCSGKLICCTTVPWNSSWSNKSQDEIWDNMTWMEWEREINNYTDIIYSLIEESQNQQEKNEQELLALD

SEQ ID NO: 54 - protein, 658 aa, Artificial Sequence
  Description of Artificial Sequence Synthetic polypeptide
MRVRGIQRNCQHLWRWGTLILGMLMICSAAENLWVTVYYGVPVWKEANTTLFCASDAKAYDTEVHNVWATHACVPTDPNPQEIVLENVTENFNMWKNNMVEQMHEDIISLWDQSLKPCVKLTPLCVTLNCTNVNVTNTTNNTEEKGEIKNCSFNITTEIRDKKQKVYALFYRLDVVPIDDNNNNSSNYRLINCNTSACTQACPKVSFEPIPIHYCAPAGFAILKCNDKKFNGTGPCKNVSTVQCTHGIKPVVSTQLLLNGSLAEEEIIIRSENITNNAKTIIVQLNESVEINCTRPNNNTRKSIRIGPGQAFYATGDIIGDIRQAHCNISGTKWNKTLQQVAKKLREHFNNKTIIFKPSSGGDLEITTHSFNCRGEFFYCNTSGLFNSTWIGNGTKNNNNTNDTITLPCRIKQIINMWQGVGQCMYAPPIEGKITCKSNITGLLLTRDGGNNNTNETEIFRPGGGDMRDNWRSELYKYKVVKIEPLGVAPTKCKERVVGRRRRRRAVGIGAVFLGFLGAAGSTMGAASITLTVQARQLLSGIVQQQSNLLRAPEAQQHLLQLTVWGIKQLQARVLAVERYLKDQQLLGIWGCSGKLICCTTVPWNSSWSNKSQDEIWDNMTWMEWEREINNYTDIIYSLIEESQNQQEKNEQELLALD

SEQ ID NO: 55 - protein, 658 aa, Artificial Sequence
  Description of Artificial Sequence Synthetic polypeptide
MRVRGIQRNCQHLWRWGTLILGMLMICSAAENLWVTVYYGVPVWKEANTTLFCASDAKAYDTEVHNVWATHACVPTDPNPQEIVLENVTENFNMWKNNMVEQMHEDIISLWDQSLKPCVKLTPLCVTLNCTNVNVTNTTNNTEEKGEIKNCSFNITTEIRDKKQKVYALFYRLDVVPIDDNNNNSSNYRLINCNTSAITQACPKVSFEPIPIHYCAPAGFAILKCNDKKFNGTGPCKNVSTVQCTHGIKPVVSTQLLLNGSLAEEEIIIRSENITNNAKTIIVQLNESVEINCTRPNNNTRKSIRIGPGQAFYATGDIIGDIRQAHCNISGTKWNKTLQQVAKKLREHFNNKTIIFKPSSGGDLEITTHSFNCRGEFFYCNTSGLFNSTWIGNGTKNNNNTNDTITLPCRIKQIINMWQGVGQAMYAPPIEGKITCKSNITGLLLTRDGGNNNTNETEIFRPGGGDMRDNWRSELYKYKVVKIEPLGVAPTKCKERVVGRRRRRRAVGIGAVFLGFLGAAGSTMGAASMTLTVQARQLLSGIVQQQSNLLRAPEAQQHLLQLTVWGIKQLQARVLAVERYLKDQQLLGIWGCSGKLICCTTVPWNSSWSNKSQDEIWDNMTWMEWEREINNYTDIIYSLIEESQNQQEKNEQELLALD

SEQ ID NO: 56 - protein, 658 aa, Artificial Sequence
  Description of Artificial Sequence Synthetic polypeptide
MRVRGIQRNCQHLWRWGTLILGMLMICSAAENLWVTVYYGVPVWKEANTTLFCASDAKAYDTKVHNVWATHACVPTDPNPQEIVLENVTENFNMWKNNMVEQMHEDIISLWDQSLKPCVKLTPLCVTLNCTNVNVTNTTNNTEEKGEIKNCSFNITTEIRDKKQKVYALFYRLDVVPIDDNNNNSSNYRLINCNTSAITQACPKVSFEPIPIHYCAPAGFAILKCNDKKFNGTGPCKNVSTVQCTHGIKPVVSTQLLLNGSLAEEEIIIRSENITNNAKTIIVQLNESVEINCTRPNNNTRKSIRIGPGQWFYATGDIIGDIRQAHCNISGTKWNKTLQQVAKKLREHFNNKTIIFKPSSGGDLEITTHSFNCRGEFFYCNTSGLFNSTWIGNGTKNNNNTNDTITLPCRIKQIINMWQGVGQAMYAPPIEGKITCKSNITGLLLTRDGGNNNTNETEIFRPGGGDMRDNWRSELYKYKVVKIEPLGVAPTKCKERVVGRRRRRRAVGIGAVFLGFLGAAGSTMGAASMTLTVQARQLLSGIVQQQSNLLRAPEAQQHLLQLTVWGIKQLQARVLAVERYLKDQQLLGIWGCSGKLICCTTVPWNSSWSNKSQDEIWDNMTWMEWEREINNYTDIIYSLIEESQNQQEKNEQELLALD

SEQ ID NO: 57 - protein, 658 aa, Artificial Sequence
  Description of Artificial Sequence Synthetic polypeptide
MRVRGIQRNCQHLWRWGTLILGMLMICSAAENLWVTVYYGVPVWKEANTTLFCASDAKAYDTEVRNVWATHACVPTDPNPQEIVLENVTENFNMWKNNMVEQMHEDIISLWDQSLKPCVKLTPLCVTLNCTNVNVTNTTNNTEEKGEIKNCSFNITTEIRDKKQKVYALFYRLDVVPIDDNNNNSSNYRLINCNTSAITQACPKVSFEPIPIHYCAPAGFAILKCNDKKFNGTGPCKNVSTVQCTHGIKPVVSTQLLLNGSLAEEEIIIRSENITNNAKTIIVQLNESVEINCTRPNNNTRKSIRIGPGQWFYATGDIIGDIRQAHCNISGTKWNKTLQQVAKKLREHFNNKTIIFKPSSGGDLEITTHSFNCRGEFFYCNTSGLFNSTWIGNGTKNNNNTNDTITLPCRIKQIINMWQGVGQAMYAPPIEGKITCKSNITGLLLTRDGGNNNTNETEIFRPGGGDMRDNWRSELYKYKVVKIEPLGVAPTKCKERVVGRRRRRRAVGIGAVFLGFLGAAGSTMGAASMTLTVQARQLLSGIVQQQSNLLRAPEAQQHLLQLTVWGIKQLQARVLAVERYLKDQQLLGIWGCSGKLICCTTVPWNSSWSNKSQDEIWDNMTWMEWEREINNYTDIIYSLIEESQNQQEKNEQELLALD

SEQ ID NO: 58 - protein, 672 aa, Artificial Sequence
  Description of Artificial Sequence Synthetic polypeptide
VDATMGSLQPLATLYLLGMLVASVLAAENLWVTVYYGVPVWKEANTTLFCASDAKAYDTEVHNVWATHACVPTDPNPQEIVLENVTENFNMWKNNMVEQMHEDIISLWDQSLKPCVKLTPLCVTLNCTNVNVTNTTNNTEEKGEIKNCSFNITTEIRDKKQKVYALFYRLDVVPIDDNNNNSSNYRLINCNTSAITQACPKVSFEPIPIHYCAPAGFAILKCNDKKFNGTGPCKNVSTVQCTHGIKPVVSTQLLLNGSLAEEEIIIRSENITNNAKTIIVQLNESVEINCTRPNNNTRKSIRIGPGQAFYATGDIIGDIRQAHCNISGTKWNKTLQQVAKKLREHFNNKTIIFKPSSGGDLEITTHSFNCRGEFFYCNTSGLFNSTWIGNGTKNNNNTNDTITLPCRIKQIINMWQGVGQAMYAPPIEGKITCKSNITGLLLTRDGGNNNTNETEIFRPGGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGSGLNDIFEAQKIEWHE

SEQ ID NO: 59 - protein, 672 aa, Artificial Sequence
  Description of Artificial Sequence Synthetic polypeptide
VDATMGSLQPLATLYLLGMLVASVLAAENLWVTVYYGVPVWKEANTTLFCASDAKAYDTEVHNVWATHACVPTDPNPQEIVLENVTENFNMWKNNMVEQMHEDIISLWDQSLKPCVKLTPLCVTLNCTNVNVTNTTNNTEEKGEIKNCSFNITTEIRDKKQKVYALFYRLDVVPIDDNNNNSSNYRLINCNTSACTQACPKVSFEPIPIHYCAPAGFAILKCNDKKFNGTGPCKNVSTVQCTHGIKPVVSTQLLLNGSLAEEEIIIRSENITNNAKTIIVQLNESVEINCTRPNNNTRKSIRIGPGQAFYATGDIIGDIRQAHCNISGTKWNKTLQQVAKKLREHFNNKTIIFKPSSGGDLEITTHSFNCRGEFFYCNTSGLFNSTWIGNGTKNNNNTNDTITLPCRIKQIINMWQGVGQCMYAPPIEGKITCKSNITGLLLTRDGGNNNTNETEIFRPGGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGSGLNDIFEAQKIEWHE

SEQ ID NO: 60 - protein, 672 aa, Artificial Sequence
  Description of Artificial Sequence Synthetic polypeptide
VDATMGSLQPLATLYLLGMLVASVLAAENLWVTVYYGVPVWKEANTTLFCASDAKAYDTKVHNVWATHACVPTDPNPQEIVLENVTENFNMWKNNMVEQMHEDIISLWDQSLKPCVKLTPLCVTLNCTNVNVTNTTNNTEEKGEIKNCSFNITTEIRDKKQKVYALFYRLDVVPIDDNNNNSSNYRLINCNTSAITQACPKVSFEPIPIHYCAPAGFAILKCNDKKFNGTGPCKNVSTVQCTHGIKPVVSTQLLLNGSLAEEEIIIRSENITNNAKTIIVQLNESVEINCTRPNNNTRKSIRIGPGQWFYATGDIIGDIRQAHCNISGTKWNKTLQQVAKKLREHFNNKTIIFKPSSGGDLEITTHSFNCRGEFFYCNTSGLFNSTWIGNGTKNNNNTNDTITLPCRIKQIINMWQGVGQAMYAPPIEGKITCKSNITGLLLTRDGGNNNTNETEIFRPGGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGSGLNDIFEAQKIEWHE

SEQ ID NO: 61 - protein, 672 aa, Artificial Sequence
  Description of Artificial Sequence Synthetic polypeptide
VDATMGSLQPLATLYLLGMLVASVLAAENLWVTVYYGVPVWKEANTTLFCASDAKAYDTEVRNVWATHACVPTDPNPQEIVLENVTENFNMWKNNMVEQMHEDIISLWDQSLKPCVKLTPLCVTLNCTNVNVTNTTNNTEEKGEIKNCSFNITTEIRDKKQKVYALFYRLDVVPIDDNNNNSSNYRLINCNTSAITQACPKVSFEPIPIHYCAPAGFAILKCNDKKFNGTGPCKNVSTVQCTHGIKPVVSTQLLLNGSLAEEEIIIRSENITNNAKTIIVQLNESVEINCTRPNNNTRKSIRIGPGQWFYATGDIIGDIRQAHCNISGTKWNKTLQQVAKKLREHFNNKTIIFKPSSGGDLEITTHSFNCRGEFFYCNTSGLFNSTWIGNGTKNNNNTNDTITLPCRIKQIINMWQGVGQAMYAPPIEGKITCKSNITGLLLTRDGGNNNTNETEIFRPGGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGSGLNDIFEAQKIEWHE

SEQ ID NO: 62 - protein, 827 aa, Artificial Sequence
  Description of Artificial Sequence Synthetic polypeptide
VTVVDASTMGSLQPLATLYLLGMLVASVLAAENLWVTVYYGVPVWKEANTTLFCASDAKAYDTEVHNVWATHACVPTDPNPQEIVLENVTENFNMWKNNMVEQMHEDIISLWDQSLKPCVKLTPLCVTLDCTNVKVTSTTSNTEEKGEIKNCSFNITTEIRDKKQKVYALFYRLDVVPIDDNNNNSSNYRLINCNTSACTQACPKVSFEPIPIHYCAPAGFAILKCNDKKFNGTGPCKNVSTVQCTHGIKPVVSTQLLLNGSLAEEEIIIRSENITNNAKTIIVQLNESVEINCTRPNNNTRKSIRIGPGQAFYATGDIIGDIRQAHCNISGTKWNKTLQQVAKKLREHFNNKTIIFKPSSGGDLEITTHSFNCRGEFFYCNTSGLFNSTWIGNGTKNNNNTNDTITLPCRIKQIINMWQGVGQCMYAPPIEGKITCKSNITGLLLTRDGGNNNTNETEIFRPGGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGGGSGDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 63 - protein, 656 aa, Artificial Sequence
  Description of Artificial Sequence Synthetic polypeptide
VDASTMGSLQPLATLYLLGMLVASVLAAENLWVTVYYGVPVWKEANTTLFCASDAKAYDTEVHNVWATHACVPTDPNPQEIVLENVTENFNMWKNNMVEQMHEDIISLWDQSLKPCVKLTPLCVTLDCTNVKVTSTTSNTEEKGEIKNCSFNITTEIRDKKQKVYALFYRLDVVPIDDNNNNSSNYRLINCNTSACTQACPKVSFEPIPIHYCAPAGFAILKCNDKKFNGTGPCKNVSTVQCTHGIKPVVSTQLLLNGSLAEEEIIIRSENITNNAKTIIVQLNESVEINCTRPNNNTRKSIRIGPGQAFYATGDIIGDIRQAHCNISGTKWNKTLQQVAKKLREHFNNKTIIFKPSSGGDLEITTHSFNCRGEFFYCNTSGLFNSTWIGNGTKNNNNTNDTITLPCRIKQIINMWQGVGQCMYAPPIEGKITCKSNITGLLLTRDGGNNNTNETEIFRPGGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALD

SEQ ID NO: 64 - protein, 656 aa, Artificial Sequence
  Description of Artificial Sequence Synthetic polypeptide
VDASTMGSLQPLATLYLLGMLVASVLAAENLWVTVYYGVPVWKEANTTLFCASDAKAYDTEVHNVWATHACVPTDPNPQEIVLENVTENFNMWKNNMVEQMHEDIISLWDQSLKPCVKLTPLCVTLNCTNVNVTSTTSNTEEKGEIKNCSFNITTEIRDKKQKVYALFYRLDVVPIDDNNNNSSNYRLINCNTSACTQACPKVSFEPIPIHYCAPAGFAILKCNDKKFNGTGPCKNVSTVQCTHGIKPVVSTQLLLNGSLAEEEIIIRSENITNNAKTIIVQLNESVEINCTRPNNNTRKSIRIGPGQAFYATGDIIGDIRQAHCNISGTKWNKTLQQVAKKLREHFNNKTIIFKPSSGGDLEITTHSFNCRGEFFYCNTSGLFNSTWIGNGTKNNNNTNDTITLPCRIKQIINMWQGVGQCMYAPPIEGKITCKSNITGLLLTRDGGNNNTNETEIFRPGGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALD

SEQ ID NO: 65 - protein, 656 aa, Artificial Sequence
  Description of Artificial Sequence Synthetic polypeptide
VDASTMGSLQPLATLYLLGMLVASVLAAENLWVTVYYGVPVWKEANTTLFCASDAKAYDTEVHNVWATHACVPTDPNPQEIVLENVTENFNMWKNNMVEQMHEDIISLWDQSLKPCVKLTPLCVTLNCTNVNVTNTTNNTEEKGEIKNCSFNITTEIRDKKQKVYALFYRLDVVPIDDNNNNSSNYRLINCNTSACTQACPKVSFEPIPIHYCAPAGFAILKCNDKKFNGTGPCKNVSTVQCTHGIKPVVSTQLLLNGSLAEEEIIIRSENITNNAKTIIVQLNESVEINCTRPNNNTRKSIRIGPGQAFYATGDIIGDIRQAHCNISGTKWNKTLQQVAKKLREHFNNKTIIFKPSSGGDLEITTHSFNCRGEFFYCNTSGLFNSTWIGNGTKNNNNTNDTITLPCRIKQIINMWQGVGQCMYAPPIEGKITCKSNITGLLLTRDGGNNNTNETEIFRPGGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALD

SEQ ID NO: 66 - protein, 672 aa, Artificial Sequence
  Description of Artificial Sequence Synthetic polypeptide
VDATMGSLQPLATLYLLGMLVASVLAAENLWVTVYYGVPVWKEANTTLFCASDAKAYDTEVHNVWATHACVPTDPNPQEIVLENVTENFNMWKNNMVEQMHEDIISLWDQSLKPCVKLTPLCVTLNCTNVNVTNTTNNTEEKGEIKNCSFNITTEIRDKKQKVYALFYRLDVVPIDDNNNNSSNYRLINCNTSACTQACPKVSFEPIPIHYCAPAGFAILKCNDKKFNGTGPCKNVSTVQCTHGIKPVVSTQLLLNGSLAEEEIIIRSENITNNAKTIIVQLNESVEINCTRPNANTRKSIRIGPGQAFYATGDIIGDIRQAHCNISGTKWNKTLQQVAKKLREHFNNKTIIFKPSSGGDLEITTHSFNCRGEFFYCNTSGLFNSTWIGNGTKNNNNTNDTITLPCRIKQIINMWQGVGQCMYAPPIEGKITCKSNITGLLLTRDGGNNNTNETEIFRPGGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGSGLNDIFEAQKIEWHE

SEQ ID NO: 67 - protein, 672 aa, Artificial Sequence
  Description of Artificial Sequence Synthetic polypeptide
VDATMGSLQPLATLYLLGMLVASVLAAENLWVTVYYGVPVWKEANTTLFCASDAKAYDTEVHNVWATHACVPTDPNPQEIVLENVTENFNMWKNNMVEQMHEDIISLWDQSLKPCVKLTPLCVTLNCTNVNVTNTTNNTEEKGEIKNCSFNITTEIRDKKQKVYALFYRLDVVPIDDNNNNSSNYRLINCNTSACTQACPKVSFEPIPIHYCAPAGFAILKCNDKKFNGTGPCKNVSTVQCTHGIKPVVSTQLLLNGSLAEEEIIIRSENITNNAKTIIVQLNESVEINCTRPNNNTRKSIRIGPGQAFYATGDIIGDIRQAHCAISGTKWNKTLQQVAKKLREHFNNKTIIFKPSSGGDLEITTHSFNCRGEFFYCNTSGLFNSTWIGNGTKNNNNTNDTITLPCRIKQIINMWQGVGQCMYAPPIEGKITCKSNITGLLLTRDGGNNNTNETEIFRPGGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGSGLNDIFEAQKIEWHE

SEQ ID NO: 68 - protein, 662 aa, Artificial Sequence
  Description of Artificial Sequence Synthetic polypeptide
VDASTMGSLQPLATLYLLGMLVASVLAAENLWVTVYYGVPVWKEANTTLFCASDAKAYDTEVHNVWATHACVPTDPNPQEIVLENVTENFNMWKNNMVEQMHEDIISLWDQSLKPCVKLTPLCVTLDCTNVKVTSTTSNTEEKGEIKNCSFNITTEIRDKKQKVYALFYRLDVVPIDDNNNNSSNYRLINCNTSACTQACPKVSFEPIPIHYCAPAGFAILKCNDKKFNGTGPCKNVSTVQCTHGIKPVVSTQLLLNGSLAEEEIIIRSENITNNAKTIIVQLNESVEINCTRPNNNTRKSIRIGPGQAFYATGDIIGDIRQAHCNISGTKWNKTLQQVAKKLREHFNNKTIIFKPSSGGDLEITTHSFNCRGEFFYCNTSGLFNSTWIGNGTKNNNNTNDTITLPCRIKQIINMWQGVGQCMYAPPIEGKITCKSNITGLLLTRDGGNNNTNETEIFRPGGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDLPSTGG

SEQ ID NO: 69 - protein, 668 aa, Artificial Sequence
  Description of Artificial Sequence Synthetic polypeptide
MGSLQPLATLYLLGMLVASVLAAENLWVTVYYGVPVWKEANTTLFCASDAKAYDTEVHNVWATHACVPTDPNPQEIVLENVTENFNMWKNNMVEQMHEDIISLWDQSLKPCVKLTPLCVTLDCTNVKVTNTTNNTEEKGEIKNCSFNITTEIRDKKQKVYALFYRLDVVPIDDNNNNSSNYRLINCNTSACTQACPKVSFEPIPIHYCAPAGFAILKCNDKKFNGTGPCKNVSTVQCTHGIKPVVSTQLLLNGSLAEEEIIIRSENITNNAKTIIVQLNESVEINCTRPNNNTRKSIRIGPGQAFYATGDIIGDIRQAHCNISGTKWNKTLQQVAKKLREHFNNKTIIFKPSSGGDLEITTHSFNCRGEFFYCNTSGLFNSTWIGNGTKNNNNTNDTITLPCRIKQIINMWQGVGQCMYAPPIEGKITCKSNITGLLLTRDGGNNNTNETEIFRPGGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGSGLNDIFEAQKIEWHE

SEQ ID NO: 70 - protein, 668 aa, Artificial Sequence
  Description of Artificial Sequence Synthetic polypeptide
MGSLQPLATLYLLGMLVASVLAAENLWVTVYYGVPVWKEANTTLFCASDAKAYDTEVHNVWATHACVPTDPNPQEIVLENVTENFNMWKNNMVEQMHEDIISLWDQSLKPCVKLTPLCVTLNCTNVNVTSTTSNTEEKGEIKNCSFNITTEIRDKKQKVYALFYRLDVVPIDDNNNNSSNYRLINCNTSACTQACPKVSFEPIPIHYCAPAGFAILKCNDKKFNGTGPCKNVSTVQCTHGIKPVVSTQLLLNGSLAEEEIIIRSENITNNAKTIIVQLNESVEINCTRPNNNTRKSIRIGPGQAFYATGDIIGDIRQAHCNISGTKWNKTLQQVAKKLREHFNNKTIIFKPSSGGDLEITTHSFNCRGEFFYCNTSGLFNSTWIGNGTKNNNNTNDTITLPCRIKQIINMWQGVGQCMYAPPIEGKITCKSNITGLLLTRDGGNNNTNETEIFRPGGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGSGLNDIFEAQKIEWHE

SEQ ID NO: 71 - protein, 668 aa, Artificial Sequence
  Description of Artificial Sequence Synthetic polypeptide
MGSLQPLATLYLLGMLVASVLAAENLWVTVYYGVPVWKEANTTLFCASDAKAYDTEVHNVWATHACVPTDPNPQEIVLENVTENFNMWKNNMVEQMHEDIISLWDQSLKPCVKLTPLCVTLDCTNVKVTSTTSNTEEKGEIKNCSFNITTEIRDKKQKVYALFYRLDVVPIDDNNNNSSNYRLINCNTSACTQACPKVSFEPIPIHYCAPAGFAILKCNDKKFNGTGPCKNVSTVQCTHGIKPVVSTQLLLNGSLAEEEIIIRSENITNNAKTIIVQLNESVEINCTRPNNNTRKSIRIGPGQAFYATGDIIGDIRQAHCNISGTKWNKTLQQVAKKLREHFNNKTIIFKPSSGGDLEITTHSFNCRGEFFYCNTSGLFNSTWIGNGTKNNNNTNDTITLPCRIKQIINMWQGVGQCMYAPPIEGKITCKSNITGLLLTRDGGNNNTNETEIFRPGGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGSGLNDIFEAQKIEWHE

SEQ ID NO: 72 - protein, 651 aa, Artificial Sequence
  Description of Artificial Sequence Synthetic polypeptide
MGSLQPLATLYLLGMLVASVLAAENLWVTVYYGVPVWKEANTTLFCASDAKAYDTKVHNVWATHACVPTDPNPQEIVLENVTENFNMWKNNMVEQMHEDIISLWDQSLKPCVKLTPLCVTLNCTNVNVTNTTNNTEEKGEIKNCSFNITTEIRDKKQKVYALFYRLDVVPIDDNNNNSSNYRLINCNTSACTQACPKVSFEPIPIHYCAPAGFAILKCNDKKFNGTGPCKNVSTVQCTHGIKPVVSTQLLLNGSLAEEEIIIRSENITNNAKTIIVQLNESVEINCTRPNNNTRKSIRIGPGQWFYATGDIIGDIRQAHCNISGTKWNKTLQQVAKKLREHFNNKTIIFKPSSGGDLEITTHSFNCRGEFFYCNTSGLFNSTWIGNGTKNNNNTNDTITLPCRIKQIINMWQGVGQCMYAPPIEGKITCKSNITGLLLTRDGGNNNTNETEIFRPGGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALD

SEQ ID NO: 73 - protein, 630 aa, Artificial Sequence
  Description of Artificial Sequence Synthetic polypeptide
KLVDTMRVRGIQRNCQHLWRWGTLILGMLMICSAAENLWVTVYYGVPVWKEANTTLFCASDAKAYDTEVHNVWATHACVPTDPNPQEIVLENVTENFNMWKNNMVEQMHEDIISLWDQSLKPCVKLTPLCVTLNCTNVNVTATTNNTEEKGEIKNCSFNITTEIRDKKQKVYALFYRLDVVPIDDNNNNSSNYRLINCNTSAITQACPKVSFEPIPIHYCAPAGFAILKCNDKKFNGTGPCKNVSTVQCTHGIKPVVSTQLLLNGSLAEEEIIIRSENITNNAKTIIVQLNESVEINCTRPNNNTRKSIRIGPGQAFYATGDIIGDIRQAHCNISGTKWNKTLQQVAKKLREHFNNKTIIFKPSSGGDLEITTHSFNCRGEFFYCNTSGLFNSTWIGNGTKNNNNTNDTITLPCRIKQIINMWQGVGQAMYAPPIEGKITCKSNITGLLLTRDGGNNNTNETEIFRPGGGDMRDNWRSELYKYKVVKIEPLGVAPTKAKLTVQARQLLSGIVQQQSNLLRAIEAQQHLLQLTVWGIKQLQARVLAVERYLKDQQLEIWDNMTWMEWEREINNYTDIIYSLIEESQNQQEKNEQELLALDKWASLWNWFDITNWLWGLNDIFEAQKIEWHE

SEQ ID NO: 74 - protein, 630 aa, Artificial Sequence
  Description of Artificial Sequence Synthetic polypeptide
KLVDTMRVRGIQRNCQHLWRWGTLILGMLMICSAAENLWVTVYYGVPVWKEANTTLFCASDAKAYDTEVHNVWATHACVPTDPNPQEIVLENVTENFNMWKNNMVEQMHEDIISLWDQSLKPCVKLTPLCVTLNCTNVNVTNTTANTEEKGEIKNCSFNITTEIRDKKQKVYALFYRLDVVPIDDNNNNSSNYRLINCNTSAITQACPKVSFEPIPIHYCAPAGFAILKCNDKKFNGTGPCKNVSTVQCTHGIKPVVSTQLLLNGSLAEEEIIIRSENITNNAKTIIVQLNESVEINCTRPNNNTRKSIRIGPGQAFYATGDIIGDIRQAHCNISGTKWNKTLQQVAKKLREHFNNKTIIFKPSSGGDLEITTHSFNCRGEFFYCNTSGLFNSTWIGNGTKNNNNTNDTITLPCRIKQIINMWQGVGQAMYAPPIEGKITCKSNITGLLLTRDGGNNNTNETEIFRPGGGDMRDNWRSELYKYKVVKIEPLGVAPTKAKLTVQARQLLSGIVQQQSNLLRAIEAQQHLLQLTVWGIKQLQARVLAVERYLKDQQLEIWDNMTWMEWEREINNYTDIIYSLIEESQNQQEKNEQELLALDKWASLWNWFDITNWLWGLNDIFEAQKIEWHE

SEQ ID NO: 75 - protein, 630 aa, Artificial Sequence
  Description of Artificial Sequence Synthetic polypeptide
KLVDTMRVRGIQRNCQHLWRWGTLILGMLMICSAAENLWVTVYYGVPVWKEANTTLFCASDAKAYDTEVHNVWATHACVPTDPNPQEIVLENVTENFNMWKNNMVEQMHEDIISLWDQSLKPCVKLTPLCVTLQCTNVQVTQTTQNTEEKGEIKNCSFNITTEIRDKKQKVYALFYRLDVVPIDDNNNNSSNYRLINCNTSAITQACPKVSFEPIPIHYCAPAGFAILKCNDKKFNGTGPCKNVSTVQCTHGIKPVVSTQLLLNGSLAEEEIIIRSENITNNAKTIIVQLNESVEINCTRPNNNTRKSIRIGPGQAFYATGDIIGDIRQAHCNISGTKWNKTLQQVAKKLREHFNNKTIIFKPSSGGDLEITTHSFNCRGEFFYCNTSGLFNSTWIGNGTKNNNNTNDTITLPCRIKQIINMWQGVGQAMYAPPIEGKITCKSNITGLLLTRDGGNNNTNETEIFRPGGGDMRDNWRSELYKYKVVKIEPLGVAPTKAKLTVQARQLLSGIVQQQSNLLRAIEAQQHLLQLTVWGIKQLQARVLAVERYLKDQQLEIWDNMTWMEWEREINNYTDIIYSLIEESQNQQEKNEQELLALDKWASLWNWFDITNWLWGLNDIFEAQKIEWHE

SEQ ID NO: 76 - protein, 646 aa, Artificial Sequence
  Description of Artificial Sequence Synthetic polypeptide
MGSLQPLATLYLLGMLVASVLAAENLWVTVYYGVPVWKEANTTLFCASDAKAYDTEVHNVWATHACVPTDPNPQEIVLENVTENFNMWKNNMVEQMHEDIISLWDQSLKPCVKLTPLCVTLNCSTATVNNRAVDEMKNCSFNITTEIRDKKQKVYALFYRLDVVPIDDNNNNSSNYRLINCNTSACTQACPKVSFEPIPIHYCAPAGFAILKCNDKKFNGTGPCKNVSTVQCTHGIKPVVSTQLLLNGSLAEEEIIIRSENITNNAKTIIVQLNESVEINCTRPNNNTRKSIRIGPGQAFYATGDIIGDIRQAHCNISGTKWNKTLQQVAKKLREHFNNKTIIFKPSSGGDLEITTHSFNCRGEFFYCNTSGLFNSTWIGNGTKNNNNTNDTITLPCRIKQIINMWQGVGQCMYAPPIEGKITCKSNITGLLLTRDGGNNNTNETEIFRPGGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALD

SEQ ID NO: 77 - protein, 827 aa, Artificial Sequence; identical to SEQ ID NO: 62
  Description of Artificial Sequence Synthetic polypeptide

SEQ ID NO: 78 - protein, 4 aa, Artificial Sequence
  Description of Artificial Sequence Synthetic peptide
GGGS

SEQ ID NO: 79 - DNA, 1947 bp, Artificial Sequence
  Description of Artificial Sequence Synthetic polynucleotide
atgggatctctgcaacctctggccacactgtacctgctgggaatgctggtggcttctgtgctggccgccgagaatctgtgggtcacagtgtactatggcgtgcccgtgtggaaagaggccaagaccacactgttctgtgccagcgacgccaaggcctacaagaaagaggtgcacaacgtctgggccacacacgcctgtgtgcctaccgatccatctcctcaagagctgttcctggaaaacgtgaccgagaacttcaacatgtggaagaacgacatggtggaccagatgcacgaggacatcatcagcctgtgggaccagagcctgaagccttgcgtgaagctgacccctctgtgcgtgaccctgatctgtagcaccgccaccgtgaacaacagagccgtggacgagatgaagaactgcagcttcaacaccaccaccgagatccgggacaagaagaagaaagagtacgccctgttctatcggagcgacgtggtgcccctggacgagacaaacaacaccagcgagtaccggctgatcaactgcaacacctccgcctgcactcaggcctgtcctaaagtgaccttcgagcccattcctatccactactgtgcccctgccggctacgccatcctgaagtgcaacaacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcattgtgcatctgaacacctctgtggaaatcgtgtgcacccggcctaacaacaacacccggaagtctgtgcggatcggccctggccagacattctatgccaccggcgatatcatcggcgacatcaagcaggcccactgcaacatcagcgaggaaaagtggaacgagacactgcagaaagtgggcatcgagctgcagaagcacttccccaacaagaccatcaagtacaaccagagcgctggcggcgacatggaaatcaccacacacagcttcaattgtggcggcgagttcttctactgcaataccagcaagctgttcaacagcacctacaacggcacctatatcagcaccaactccaccaatagcaccagctacatcaccctgcagtgccggatcaagcagatcatcaatatgtggcaaggcgtcggccggtgtatgtacgcccctcctatcgccggcaacatcacctgtcggagcaatatcacaggcctgctgctcaccagagatggcggcatcaacaacgtgtccaacgagacagaaaccttccggcctgccggcggagacatgagagacaattggagaagcgagctgtacaagtacaaggtggtcaagatcgagcccctgggcgtcgcaccaacacggtgcaagagaagagtcgtgggccgtcgtagaaggcggagagccgttggaattggcgccgtgttcctgggctttctgggagccgctggatctacaatgggcgctgccagcatgaccctgacagtgcaggctagaaatctgctgagcggcatcgtgcagcagcagagcaatctgctcagagcccctgaggctcagcagcacctcctgaaactgacagtgtggggaatcaagcagctgcaggccagagtgctggcagtggaaagatacctgagggaccagcagctcctcggaatctggggatgtagcggcaagctgatctgctgcaccaacgtgccctggaactccagctggtccaaccggaatctgagcgagatctgggataacatgacctggctgcagtgggacaaagagatcagcaactacacccagatcatctacggcctgctggaagagagccagaaccagcaagagaaaaacgagcaggacctgctggccctggactgataa

SEQ ID NO: 80 - DNA, 1932 bp, Artificial Sequence
  Description of Artificial Sequence Synthetic polynucleotide
atgggatctctgcagcctctggccacactgtacctgctgggaatgctggtggcctcttgtctgggcgttgagaagctgtgggtcaccgtgtactatggcgtgcccgtgtggaaagaagcctgcaccacactgttctgtgccagcgacgccaaggcctacgataccaaggtgcgcaatgtgtgggccactcactgctgcgtgcccaccgatcctaatcctcaagaggtggtgctggaaaacgtgaccgagcacttcaacatgtggaagaacaacatggtcgagcagatgcaagaggacatcatcagcctgtgggaccagagcctgaagccttgcgtgaagctgacccctctgtgcgtgaccctgaactgtagcaccgccaccgtgaacaacagagccgtggacgagatgaagaactgcagcttcaacatcaccacctccatcagagacaaggtgcagaaagagtacgccctgttctacaagctggacgtggtgcccatcgacaacaacaacaccagctacagactgatcagctgcgacaccagcgtgatcacccaggcctgtcctaagatcagcttcgagcccattcctatccactactgtgcccctgccggcttcgccatcctgaagtgcaacgacaagaccttcaacggcaagggcccctgcaagaacgtgtccaccgtgcagtgtacccacggcatcagaccagtggtgtctacccagctgctgctgaatggctctctggccgaggaagaagtggtcatcagaagcgacaacttcaccaacaacgccaagaccatcatcgtgcagctgaaagagagcgtcgagatcaactgcacccggcctaacaacaatacccggaagtccatccacatcggccctggcagatggttttacaccaccggcgagattatcggcgacatcagacaggcccactgcaacatcagccgggccaagtggaacgacaccctgaagcagatcgtgatcaagctgagagagcagttcgagaacaagacgatcgtgttcaaccacagctctggcggcgaccccgagattgtgatgcactcctttaactgtggcggcgagttcttctactgcaacagcacccagctgttcaactccacctggaacaacaacacagagggcagcaacaataccgagggcaacaccatcacactgccctgccggatcaagcagatcatcaatatgtggcaagagatcggcaaggctatgtacgcccctcctatccggggccagatcagatgcagcagcaatatcacaggcctgctgctcaccagagatggcggcatcaacgagaacggcaccgagatcttcagacccggcggaggcgacatgcgggacaattggagaagcgagctgtacaagtacaaggtggtcaagatcgagcccctgggcgtcgcccctaccaagtgcaagagaagagtcgtgggccgtagaaggcggcggagagctgttggaattggcgccgtgttcctgggctttctgggagccgctggatctacaatgggcgctgccagcatgaccctgacagtgcaggctagacagctgctgtccggcattgtccagcagcagaacaactgcctgagagcccctgagtgtcaacaaagaatgctgcagctgaccgtgtggggcatcaaacagctgcaggccagagttctggccgtggaaagatacctgggcgaccagcagctcctcggcatctggggatgttctggcaagctgatttgctgcaccgccgtgccttggaatgccagctggtctaacaagagcctggaccggatctggaacaatatgacctggatggaatgggagcgcgagatcgacaactacacctccgagatctacaccctgatcgaggaaagccagaaccagcaagagaaaaacgagcaagagctgctcgagctggattaatga

SEQ ID NO: 81 - DNA, 1944 bp, Artificial Sequence
  Description of Artificial Sequence Synthetic polynucleotide
atgggctcgctccagccgctcgcgacgctgtacctcctgggcatgctcgtggcgtccgtgctggcggccgagaacctgtgggtgacggtgtactacggcgtgcccgtgtggaaggaggccaacaccacgctgttctgcgccagcgacgccaaggcctacgacaccgaggtgcacaacgtgtgggcgacccacgcctgcgtgccgacggaccccaacccccaggagatcgtgctggagaacgtgaccgagaacttcaacatgtggaagaacaacatggtggagcagatgcacgaggacatcatctcgctgtgggaccagtccctgaagccgtgcgtgaagctgacgcccctgtgcgtgaccctgaactgtagcaccgccaccgtgaacaacagagccgtggacgagatgaagaactgctccttcaacatcaccaccgagatccgcgacaagaagcagaaggtgtacgcgctgttctaccggctggacgtggtgccgatcgacgacaacaacaacaactccagcaactaccgcctgatcaactgcaacaccagcgcctgcacccaggcctgcccgaaggtgtccttcgagcccatccccatccactactgcgcgccggccggcttcgccatcctgaagtgcaacgacaagaagttcaacggcaccggcccctgcaagaacgtgtccaccgtgcagtgcacccacgggatcaagcccgtggtgtccacgcagctgctgctgaacggctccctggccgaggaggagatcatcatccgctccgagaacatcacgaacaacgccaagaccatcatcgtgcagctgaacgagtccgtggagatcaactgcaccaggcccaacaacaacacccgcaagtccatccggatcggccctggccaggcgttctacgccaccggcgacatcatcggcgacatccgccaggcgcactgcaacatctcgggcacgaagtggaacaagaccctgcagcaggtggcgaagaagctgcgcgagcacttcaacaacaagaccatcatcttcaagcccagctccggcggcgacctggagatcacgacccactccttcaactgccgcggcgagttcttctactgcaacacctccggcctgttcaactcgacgtggatcgggaacggcacgaagaacaacaacaacaccaacgacaccatcaccctgccctgccgcatcaagcagatcatcaacatgtggcagggcgtgggccagtgcatgtacgcgccgcccatcgagggcaagatcacctgcaagtccaacatcaccggcctgctcctgacgcgcgacggcggcaacaacaacaccaacgagaccgagatcttcaggccgggcggcggcgacatgcgcgacaactggcgctcggagctgtacaagtacaaggtggtgaagatcgagcccctgggcgtggcgccgacgcgctgcaagagacgcgtggtgggccgcagacgaaggagacgggccgtgggcatcggcgcggtgttcctgggcttcctgggagcagctggttcgacgatgggcgcagcttccatgaccctgacagtgcaggcacgcaacctgctctccggcatcgtccagcagcagtcgaacctgcttcgagcccccgaggcgcagcagcacctcctcaagctgaccgtgtggggcatcaagcagctgcaggcacgcgtgctagccgtggagcgctacctccgcgaccagcagctgctcggaatctggggctgctcgggcaagctgatctgctgcaccaacgtgccgtggaacagctcctggtccaaccgcaacctctcggagatctgggacaacatgacctggctccagtgggacaaggagatctcgaactacacccagatcatctacggcctgctggaggagtcccagaaccagcaggagaagaacgagcaggacctgctggccctggactgataa

SEQ ID NO: 82 - DNA, 1947 bp, Artificial Sequence; identical to SEQ ID NO: 79
  Description of Artificial Sequence Synthetic polynucleotide

SEQ ID NO: 83 - DNA, 1947 bp, Artificial Sequence
  Description of Artificial Sequence Synthetic polynucleotide
atgggatctctgcaacctctggccacactgtacctgctgggaatgctggtggcttctgtgctggccgccgagaatctgtgggtcacagtgtactatggcgtgcccgtgtggaaagaggccaagaccacactgttctgtgccagcgacgccaaggcctacaagaaagaggtgcacaacgtctgggccacacacgcctgtgtgcctaccgatccatctcctcaagagctgttcctggaaaacgtgaccgagaacttcaacatgtggaagaacgacatggtggaccagatgcacgaggacatcatcagcctgtgggaccagagcctgaagccttgcgtgaagctgacccctctgtgcgtgaccctgatctgtagcaccgccaccgtgaacaacagagccgtggacgagatgaagaactgcagcttcaacaccaccaccgagatccgggacaagaagaagaaagagtacgccctgttctatcggagcgacgtggtgcccctggacgagacaaacaacaccagcgagtaccggctgatcaactgcaacacctccgcctgcactcaggcctgtcctaaagtgaccttcgagcccattcctatccactactgtgcccctgccggctacgccatcctgaagtgcaacgacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcattgtgcatctgcacacccctgtggaaatcgtgtgcacccggcctaacaacaacacccggaagtctgtgcggatcggccctggccagacattctatgccaccggcgatatcatcggcgacatcaagcaggcccactgcaacatcagcgaggaaaagtggaacgagacactgcagaaagtgggcatcgagctgcagaagcacttccccaacaagaccatcaagtacaaccagagcgctggcggcgacatggaaatcaccacacacagcttcaattgtggcggcgagttcttctactgcaataccagcaagctgttcaacagcacctacaacggcacctatatcagcaccaactccaccaatagcaccagctacatcaccctgcagtgccggatcaagcagatcatcaatatgtggcaaggcgtcggccggtgtatgtacgcccctcctatcgccggcaacatcacctgtcggagcaatatcacaggcctgctgctcaccagagatggcggcatcaacaacgtgtccaacgagacagaaaccttccggcctgccggcggagacatgagagacaattggagaagcgagctgtacaagtacaaggtggtcaagatcgagcccctgggcgtcgcaccaacacggtgcaagagaagagtcgtgggccgtcgtagaaggcggagagccgttggaattggcgccgtgttcctgggctttctgggagccgctggatctacaatgggcgctgccagcatgaccctgacagtgcaggctagaaatctgctgagcggcatcgtgcagcagcagagcaatctgctcagagcccctgaggctcagcagcacctcctgaaactgacagtgtggggaatcaagcagctgcaggccagagtgctggcagtggaaagatacctgagggaccagcagctcctcggaatctggggatgtagcggcaagctgatctgctgcaccaacgtgccctggaactccagctggtccaaccggaatctgagcgagatctgggataacatgacctggctgcagtgggacaaagagatcagcaactacacccagatcatctacggcctgctggaagagagccagaaccagcaagagaaaaacgagcaggacctgctggccctggactgataa

SEQ ID NO: 84 - DNA, 2559 bp, Artificial Sequence
  Description of Artificial Sequence Synthetic polynucleotide
atgagagtgacggggatactgaggaattatccacaatggtggatatggggcatcttaggcttttggatgctaatgacttgtaatggggaaggaaacttgtgggtcacagtctactatggggtaccagtatggaaagaagcaaaaactactctgttttgtgcatcagatgccaaagcgtataagaaagaagtgcataatgtctgggcgacacatgcctgtgtacccacagaccccagcccacaagaactgtttttggaaaatgtaacagaaaattttaacatgtggaaaaatgatatggtagatcagatgcatgaggatataatcagtctatgggatcaaagcctaaagccatgtgtaaagttgaccccactctgtgtcactttaatttgtagtactgctactgttaacaatagagcagttgatgaaatgaaaaattgctccttcaatacaaccacagaaataagagataagaaaaagaaggaatatgcacttttttatagatctgatgtagtaccacttgatgaaacaaacaataccagtgagtatagattaataaattgtaatacctcagccgtaacacaagcctgtccaaaggtcacttttgaaccaattcctatacattattgtgctccagctggttatgcgattctaaagtgcaatgatgagacattcaatggaacaggaccatgcagtaatgtcagcacagtacaatgtacacatggaattaggccagtggtatcaacccaactactgttaaatggtagtctggcagaaaaagagatagtaattagatctgaaaacctgacaaacaatgccaaaataataatagtccatcttcacacccctgtagaaattgtgtgtacaaggcccggccataatacaaggaaaagtgtgagaataggcccaggacaaacattctatgcaacaggagacataataggagatataagacaagcacattgtaacattaatgaaagtaaatggaatgaaactttacaaaaggtaggtatagaattgcaaaaacacttccctaataagacaataaagtataaccaatccgcaggaggagacatggaaattacaacacatagctttaattgtggaggagaatttttctattgcaatacatcaaagctgtttaatagtacatacaatggtacatacataagtacaaacagcacaaacagtacttcatacatcacgcttcaatgcagaataaaacaaattataaacatgtggcagggggtaggaagagcaatgtatgctcctcccattgcaggaaacataacatgtagatcaaatatcacagggctactattgacacgtgatggagggatcaataatgttagcaacgagacagagacattcaggcctgcaggaggagatatgagggataattggagaagtgaattatataaatataaagtagtagaagttcagccattaggaatagcaccaactggtgcaaaaaggagagtggtggagagagaaaaaagagcagcaggactaggagctttgttccttgggttcttgggagcagcaggaagcactatgggcgcagcatcaataacgctgacggtacaggccagacaattgttgtctggtatagtgcaacagcaaagcaatttgctgagggctatagaggcgcaacagcatatgttgcaactcacggtctggggcatcaagcagctccaggcaagagtcctggctctggaaagatacctaaaggatcaacagctcctagggatgtggggctgctctggaaaactcatctgcaccactaatgtgccttggaacactagttggagtaataaatctgaaatggatatttggaataacatgacatggatgcagtgggaaagagaaattagcaattacacagagacaatatacatgttgcttgaagactcgcaacgccagcaggagagaaatgaaaaagatttactagcattggacagttggaacagtctgtggaattggtttaacataacaaactggctgtggtatataaaaatattcataatgatagtagggggcttgataggtttaagaatagtttttgctgtgctatctatagtgaatagagtcaggcagggatactcacctttgtcgttgcagacccttaccccaaacccgagggaacccgacaggctcagaggaatcgaagaagaaggtggagagcaagacagagacagatccattcgattagtgagcggattcttgccaattgtctgggacgacctgcggagcctgtgcctcttcagttaccaccgattgagagactttctattgctggcagcgagagtggtggaacttctgggacacagcagtctcaggggactgcagagggggtgggaagtccttaagtatctgggaagtcttgtgcagtattggggtctggaactaaaaaggagtgctattagtctttttgataccctagcaatagcagtagctgaaggaacagataggattatagaattaatacaaggattttgtagagctatccgcaacatacctacaagaataagacaaggctttgaagcatctttgctataataa

SEQ ID NO: 85 - DNA, 1947 bp, Artificial Sequence
  Description of Artificial Sequence Synthetic polynucleotide
atgggatctctgcagcctctggccacactgtacctgctgggaatgctggtggcttctgtgctggccgccgagaatctgtgggtcacagtgtactatggcgtgcccgtgtggaaagaggccaagaccacactgttctgtgccagcgacgccaaggcctacaagaaagaggtgcacaacgtctgggccacacacgcctgtgtgcctaccgatccatctcctcaagagctgttcctggaaaacgtgaccgagaacttcaacatgtggaagaacgacatggtggaccagatgcacgaggacatcatcagcctgtgggaccagagcctgaagccttgcgtgaagctgacccctctgtgcgtgaccctgatctgtagcaccgccaccgtgaacaacagagccgtggacgagatgaagaactgcagcttcaacaccaccaccgagatccgggacaagaagaagaaagagtacgccctgttctatcggagcgacgtggtgcccctggacgagacaaacaacaccagcgagtaccggctgatcaactgcaacacctccgcctgcacacaggcttgccccaaagtgaccttcgagcccattcctatccactactgtgcccctgccggctacgccatcctgaagtgcaacgacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcattgtgcatctgcacacccctgtggaaatcgtgtgcaccagacctggccacaacacccggaagtctgtgcggattggacccggccagaccttttatgccaccggcgatatcatcggcgacatcagacaggcccactgtaacatcaacgagagcaagtggaacgagacactgcagaaagtgggcatcgagctgcagaagcacttccccaacaagaccatcaagtacaaccagagcgctggcggcgacatggaaatcaccacacacagcttcaattgtggcggcgagttcttctactgcaataccagcaagctgttcaacagcacctacaacggcacctatatcagcaccaactccaccaatagcaccagctacatcaccctgcagtgccggatcaagcagatcatcaatatgtggcaaggcgtgggcagatgcatgtacgcccctcctatcgccggcaacatcacctgtcggagcaatatcacaggcctgctgctcaccagagatggcggcatcaacaacgtgtccaacgagacagaaaccttccggcctgccggcggagacatgagagacaattggagaagcgagctgtacaagtacaaggtggtcaagatcgagcccctgggcgtcgcaccaacacggtgcaagagaagagtcgtgggacgtagacgaaggcggagagccgttggaatcggagccgtgttcctgggctttctgggagccgctggatctacaatgggcgctgccagcatgaccctgacagtgcaggctagaaatctgctgagcggcatcgtgcagcagcagagcaatctgctcagagcccctgaggctcagcagcacctcctgaaactgacagtgtggggcatcaagcagctgcaggcaagagtgctggcagtggaaagatacctgcgggaccagcagctcctcggaatctggggatgtagcggcaagctgatctgctgcaccaacgtgccctggaactccagctggtccaaccggaatctgagcgagatctgggataacatgacctggctgcagtgggacaaagagatcagcaactacacccagatcatctacggcctgctggaagagagccagaaccagcaagagaaaaacgagcaggacctgctggccctggactgataa

SEQ ID NO: 86 - DNA, 1947 bp, Artificial Sequence
  Description of Artificial Sequence Synthetic polynucleotide
atgggatctctgcagcctctggccacactgtacctgctgggaatgctggtggcttctgtgctggccgccgagaatctgtgggtcacagtgtactatggcgtgcccgtgtggaaagaggcctgtaccacactgttctgtgccagcgacgccaaggcctacaagaaaaaggtgcgcaacgtctgggccacacactgctgtgtgcctaccgatccatctcctcaagagctgttcctggaaaacgtgaccgagaacttcaacatgtggaagaacgacatggtggaccagatgcacgaggacatcatcagcctgtgggaccagagcctgaagccttgcgtgaagctgacccctctgtgcgtgaccctgatctgtagcaccgccaccgtgaacaacagagccgtggacgagatgaagaactgcagcttcaacaccaccaccgagatccgggacaagaagaagaaagagtacgccctgttctatcggagcgacgtggtgcccctggacgagacaaacaacaccagcgagtaccggctgatcaactgcaacacctccgccgtgacacaggcttgccccaaagtgaccttcgagcccattcctatccactactgtgcccctgccggctacgccatcctgaagtgcaacgacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcattgtgcatctgcacacccctgtggaaatcgtgtgcaccagacctggccacaacacccggaagtctgtgcggattggacccggccagtggttttatgccaccggcgatatcatcggcgacatcagacaggcccactgtaacatcaacgagagcaagtggaacgagacactgcagaaagtgggcatcgagctgcagaagcacttccccaacaagaccatcaagtacaaccagagcgctggcggcgacatggaaatcaccacacacagcttcaattgtggcggcgagttcttctactgcaataccagcaagctgttcaacagcacctacaacggcacctatatcagcaccaactccaccaatagcaccagctacatcaccctgcagtgccggatcaagcagatcatcaatatgtggcaaggcgtgggcagagctatgtacgcccctcctatcgccggcaacatcacctgtcggagcaatatcacaggcctgctgctcaccagagatggcggcatcaacaacgtgtccaacgagacagaaaccttccggcctgccggcggagacatgagagacaattggagaagcgagctgtacaagtacaaggtggtcaagatcgagcccctgggcgtcgcaccaacacggtgcaagagaagagtcgtgggacgtagacgaaggcggagagccgttggaatcggagccgtgttcctgggctttctgggagccgctggatctacaatgggcgctgccagcatgaccctgacagtgcaggctagaaatctgctgagcggcatcgtgcagcagcagagcaattgcctcagagcccctgagtgtcagcagcacctcctgaaactgacagtgtggggcatcaagcagctgcaggcaagagtgctggcagtggaaagatacctgcgggaccagcagctcctcggaatctggggatgtagcggcaagctgatctgctgcaccaacgtgccctggaactccagctggtccaaccggaatctgagcgagatctgggataacatgacctggctgcagtgggacaaagagatcagcaactacacccagatcatctacggcctgctggaagagagccagaaccagcaagagaaaaacgagcaggacctgctggccctggactgataa

SEQ ID NO: 87 - protein, 647 aa, Artificial Sequence
  Description of Artificial Sequence Synthetic polypeptide
MGSLQPLATLYLLGMLVASVLAAENLWVTVYYGVPVWKEAKTTLFCASDAKAYKKEVHNVWATHACVPTDPSPQELFLENVTENFNMWKNDMVDQMHEDIISLWDQSLKPCVKLTPLCVTLICSTATVNNRAVDEMKNCSFNTTTEIRDKKKKEYALFYRSDVVPLDETNNTSEYRLINCNTSACTQACPKVTFEPIPIHYCAPAGYAILKCNNETFNGTGPCSNVSTVQCTHGIRPVVSTQLLLNGSLAEKEIVIRSENLTNNAKIIIVHLNTSVEIVCTRPNNNTRKSVRIGPGQTFYATGDIIGDIKQAHCNISEEKWNETLQKVGIELQKHFPNKTIKYNQSAGGDMEITTHSFNCGGEFFYCNTSKLFNSTYNGTYISTNSTNSTSYITLQCRIKQIINMWQGVGRCMYAPPIAGNITCRSNITGLLLTRDGGINNVSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALD

SEQ ID NO: 88 - protein, 642 aa, Artificial Sequence
  Description of Artificial Sequence Synthetic polypeptide
MGSLQPLATLYLLGMLVASCLGVEKLWVTVYYGVPVWKEACTTLFCASDAKAYDTKVRNVWATHCCVPTDPNPQEVVLENVTEHFNMWKNNMVEQMQEDIISLWDQSLKPCVKLTPLCVTLNCSTATVNNRAVDEMKNCSFNITTSIRDKVQKEYALFYKLDVVPIDNNNTSYRLISCDTSVITQACPKISFEPIPIHYCAPAGFAILKCNDKTFNGKGPCKNVSTVQCTHGIRPVVSTQLLLNGSLAEEEVVIRSDNFTNNAKTIIVQLKESVEINCTRPNNNTRKSIHIGPGRWFYTTGEIIGDIRQAHCNISRAKWNDTLKQIVIKLREQFENKTIVFNHSSGGDPEIVMHSFNCGGEFFYCNSTQLFNSTWNNNTEGSNNTEGNTITLPCRIKQIINMWQEIGKAMYAPPIRGQIRCSSNITGLLLTRDGGINENGTEIFRPGGGDMRDNWRSELYKYKVVKIEPLGVAPTKCKRRVVGRRRRRRAVGIGAVFLGFLGAAGSTMGAASMTLTVQARQLLSGIVQQQNNCLRAPECQQRMLQLTVWGIKQLQARVLAVERYLGDQQLLGIWGCSGKLICCTAVPWNASWSNKSLDRIWNNMTWMEWEREIDNYTSEIYTLIEESQNQQEKNEQELLELD

SEQ ID NO: 89 - protein, 646 aa, Artificial Sequence; identical to SEQ ID NO: 76
  Description of Artificial Sequence Synthetic polypeptide

SEQ ID NO: 90 - protein, 647 aa, Artificial Sequence; identical to SEQ ID NO: 87
  Description of Artificial Sequence Synthetic polypeptide

SEQ ID NO: 91 - protein, 647 aa, Artificial Sequence
  Description of Artificial Sequence Synthetic polypeptide
MGSLQPLATLYLLGMLVASVLAAENLWVTVYYGVPVWKEAKTTLFCASDAKAYKKEVHNVWATHACVPTDPSPQELFLENVTENFNMWKNDMVDQMHEDIISLWDQSLKPCVKLTPLCVTLICSTATVNNRAVDEMKNCSFNTTTEIRDKKKKEYALFYRSDVVPLDETNNTSEYRLINCNTSACTQACPKVTFEPIPIHYCAPAGYAILKCNDETFNGTGPCSNVSTVQCTHGIRPVVSTQLLLNGSLAEKEIVIRSENLTNNAKIIIVHLHTPVEIVCTRPNNNTRKSVRIGPGQTFYATGDIIGDIKQAHCNISEEKWNETLQKVGIELQKHFPNKTIKYNQSAGGDMEITTHSFNCGGEFFYCNTSKLFNSTYNGTYISTNSTNSTSYITLQCRIKQIINMWQGVGRCMYAPPIAGNITCRSNITGLLLTRDGGINNVSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALD

SEQ ID NO: 92 - protein, 851 aa, Artificial Sequence
  Description of Artificial Sequence Synthetic polypeptide
MRVTGILRNYPQWWIWGILGFWMLMTCNGEGNLWVTVYYGVPVWKEAKTTLFCASDAKAYKKEVHNVWATHACVPTDPSPQELFLENVTENFNMWKNDMVDQMHEDIISLWDQSLKPCVKLTPLCVTLICSTATVNNRAVDEMKNCSFNTTTEIRDKKKKEYALFYRSDVVPLDETNNTSEYRLINCNTSAVTQACPKVTFEPIPIHYCAPAGYAILKCNDETFNGTGPCSNVSTVQCTHGIRPVVSTQLLLNGSLAEKEIVIRSENLTNNAKIIIVHLHTPVEIVCTRPGHNTRKSVRIGPGQTFYATGDIIGDIRQAHCNINESKWNETLQKVGIELQKHFPNKTIKYNQSAGGDMEITTHSFNCGGEFFYCNTSKLFNSTYNGTYISTNSTNSTSYITLQCRIKQIINMWQGVGRAMYAPPIAGNITCRSNITGLLLTRDGGINNVSNETETFRPAGGDMRDNWRSELYKYKVVEVQPLGIAPTGAKRRVVEREKRAAGLGALFLGFLGAAGSTMGAASITLTVQARQLLSGIVQQQSNLLRAIEAQQHMLQLTVWGIKQLQARVLALERYLKDQQLLGMWGCSGKLICTTNVPWNTSWSNKSEMDIWNNMTWMQWEREISNYTETIYMLLEDSQRQQERNEKDLLALDSWNSLWNWFNITNWLWYIKIFIMIVGGLIGLRIVFAVLSIVNRVRQGYSPLSLQTLTPNPREPDRLRGIEEEGGEQDRDRSIRLVSGFLPIVWDDLRSLCLFSYHRLRDFLLLAARVVELLGHSSLRGLQRGWEVLKYLGSLVQYWGLELKRSAISLFDTLAIAVAEGTDRIIELIQGFCRAIRNIPTRIRQGFEASLL

SEQ ID NO: 93 - protein, 647 aa, Artificial Sequence
  Description of Artificial Sequence Synthetic polypeptide
MGSLQPLATLYLLGMLVASVLAAENLWVTVYYGVPVWKEAKTTLFCASDAKAYKKEVHNVWATHACVPTDPSPQELFLENVTENFNMWKNDMVDQMHEDIISLWDQSLKPCVKLTPLCVTLICSTATVNNRAVDEMKNCSFNTTTEIRDKKKKEYALFYRSDVVPLDETNNTSEYRLINCNTSACTQACPKVTFEPIPIHYCAPAGYAILKCNDETFNGTGPCSNVSTVQCTHGIRPVVSTQLLLNGSLAEKEIVIRSENLTNNAKIIIVHLHTPVEIVCTRPGHNTRKSVRIGPGQTFYATGDIIGDIRQAHCNINESKWNETLQKVGIELQKHFPNKTIKYNQSAGGDMEITTHSFNCGGEFFYCNTSKLFNSTYNGTYISTNSTNSTSYITLQCRIKQIINMWQGVGRCMYAPPIAGNITCRSNITGLLLTRDGGINNVSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALD

SEQ ID NO: 94 - protein, 647 aa, Artificial Sequence
  Description of Artificial Sequence Synthetic polypeptide
MGSLQPLATLYLLGMLVASVLAAENLWVTVYYGVPVWKEACTTLFCASDAKAYKKKVRNVWATHCCVPTDPSPQELFLENVTENFNMWKNDMVDQMHEDIISLWDQSLKPCVKLTPLCVTLICSTATVNNRAVDEMKNCSFNTTTEIRDKKKKEYALFYRSDVVPLDETNNTSEYRLINCNTSAVTQACPKVTFEPIPIHYCAPAGYAILKCNDETFNGTGPCSNVSTVQCTHGIRPVVSTQLLLNGSLAEKEIVIRSENLTNNAKIIIVHLHTPVEIVCTRPGHNTRKSVRIGPGQWFYATGDIIGDIRQAHCNINESKWNETLQKVGIELQKHFPNKTIKYNQSAGGDMEITTHSFNCGGEFFYCNTSKLFNSTYNGTYISTNSTNSTSYITLQCRIKQIINMWQGVGRAMYAPPIAGNITCRSNITGLLLTRDGGINNVSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNCLRAPECQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALD

SEQ ID NO: 95 - protein, 651 aa, Artificial Sequence
  Description of Artificial Sequence Synthetic polypeptide
MGSLQPLATLYLLGMLVASVLAAENLWVTVYYGVPVWKEANTTLFCASDAKAYDTEVHNVWATHACVPTDPNPQEIVLENVTENFNMWKNNMVEQMHEDIISLWDQSLKPCVKLTPLCVTLDCTNVKVTSTTSNTEEKGEIKNCSFNITTEIRDKKQKVYALFYRLDVVPIDDNNNNSSNYRLINCNTSACTQACPKVSFEPIPIHYCAPAGFAILKCNDKKFNGTGPCKNVSTVQCTHGIKPVVSTQLLLNGSLAEEEIIIRSENITNNAKTIIVQLNESVEINCTRPNNNTRKSIRIGPGQAFYATGDIIGDIRQAHCNISGTKWNKTLQQVAKKLREHFNNKTIIFKPSSGGDLEITTHSFNCRGEFFYCNTSGLFNSTWIGNGTKNNNNTNDTITLPCRIKQIINMWQGVGQCMYAPPIEGKITCKSNITGLLLTRDGGNNNTNETEIFRPGGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALD

SEQ ID NO: 96 - protein, 651 aa, Artificial Sequence
  Description of Artificial Sequence Synthetic polypeptide
MGSLQPLATLYLLGMLVASVLAAENLWVTVYYGVPVWKEANTTLFCASDAKAYDTEVHNVWATHACVPTDPNPQEIVLENVTENFNMWKNNMVEQMHEDIISLWDQSLKPCVKLTPLCVTLNCTNVNVTSTTSNTEEKGEIKNCSFNITTEIRDKKQKVYALFYRLDVVPIDDNNNNSSNYRLINCNTSACTQACPKVSFEPIPIHYCAPAGFAILKCNDKKFNGTGPCKNVSTVQCTHGIKPVVSTQLLLNGSLAEEEIIIRSENITNNAKTIIVQLNESVEINCTRPNNNTRKSIRIGPGQAFYATGDIIGDIRQAHCNISGTKWNKTLQQVAKKLREHFNNKTIIFKPSSGGDLEITTHSFNCRGEFFYCNTSGLFNSTWIGNGTKNNNNTNDTITLPCRIKQIINMWQGVGQCMYAPPIEGKITCKSNITGLLLTRDGGNNNTNETEIFRPGGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALD

SEQ ID NO: 97 - protein, 655 aa, Artificial Sequence
  Description of Artificial Sequence Synthetic polypeptide
MPMGSLQPLATLYLLGMLVASVLAAENLWVTVYYGVPVWKEAKTTLFCASDAKAYKKEVHNVWATHACVPTDPSPQELFLENVTENFNMWKNDMVDQMHEDIISLWDQSLKPCVKLTPLCVTLICSTATVNNRAVDEMKNCSFNTTTEIRDKKKKEYALFYRSDVVPLDETNNTSEYRLINCNTSACTQACPKVTFEPIPIHYCAPAGYAILKCNDETFNGTGPCSNVSTVQCTHGIRPVVSTQLLLNGSLAEKEIVIRSENLTNNAKIIIVHLHTPVEIVCTRPNNNTRKSVRIGPGQTFYATGDIIGDIKQAHCNISEEKWNETLQKVGIELQKHFPNKTIKYNQSAGGDMEITTHSFNCGGEFFYCNTSKLFNSTYNGTYISTNSTNSTSYITLQCRIKQIINMWQGVGRCMYAPPIAGNITCRSNITGLLLTRDGGINNVSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDLPSTGG

SEQ ID NO: 98 - DNA, 1989 bp, Artificial Sequence
  Description of Artificial Sequence Synthetic polynucleotide
gtcgacgccaccatgcccatgggatctctgcaacctctggccacactgtacctgctgggaatgctggtggcttctgtgctggccgccgagaatctgtgggtcacagtgtactatggcgtgcccgtgtggaaagaggccaagaccacactgttctgtgccagcgacgccaaggcctacaagaaagaggtgcacaacgtctgggccacacacgcctgtgtgcctaccgatccatctcctcaagagctgttcctggaaaacgtgaccgagaacttcaacatgtggaagaacgacatggtggaccagatgcacgaggacatcatcagcctgtgggaccagagcctgaagccttgcgtgaagctgacccctctgtgcgtgaccctgatctgtagcaccgccaccgtgaacaacagagccgtggacgagatgaagaactgcagcttcaacaccaccaccgagatccgggacaagaagaagaaagagtacgccctgttctatcggagcgacgtggtgcccctggacgagacaaacaacaccagcgagtaccggctgatcaactgcaacacctccgcctgcactcaggcctgtcctaaagtgaccttcgagcccattcctatccactactgtgcccctgccggctacgccatcctgaagtgcaacgacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcattgtgcatctgcacacccctgtggaaatcgtgtgcacccggcctaacaacaacacccggaagtctgtgcggatcggccctggccagacattctatgccaccggcgatatcatcggcgacatcaagcaggcccactgcaacatcagcgaggaaaagtggaacgagacactgcagaaagtgggcatcgagctgcagaagcacttccccaacaagaccatcaagtacaaccagagcgctggcggcgacatggaaatcaccacacacagcttcaattgtggcggcgagttcttctactgcaataccagcaagctgttcaacagcacctacaacggcacctatatcagcaccaactccaccaatagcaccagctacatcaccctgcagtgccggatcaagcagatcatcaatatgtggcaaggcgtcggccggtgtatgtacgcccctcctatcgccggcaacatcacctgtcggagcaatatcacaggcctgctgctcaccagagatggcggcatcaacaacgtgtccaacgagacagaaaccttccggcctgccggcggagacatgagagacaattggagaagcgagctgtacaagtacaaggtggtcaagatcgagcccctgggcgtcgcaccaacacggtgcaagagaagagtcgtgggccgtcgtagaaggcggagagccgttggaattggcgccgtgttcctgggctttctgggagccgctggatctacaatgggcgctgccagcatgaccctgacagtgcaggctagaaatctgctgagcggcatcgtgcagcagcagagcaatctgctcagagcccctgaggctcagcagcacctcctgaaactgacagtgtggggaatcaagcagctgcaggccagagtgctggcagtggaaagatacctgagggaccagcagctcctcggaatctggggatgtagcggcaagctgatctgctgcaccaacgtgccctggaactccagctggtccaaccggaatctgagcgagatctgggataacatgacctggctgcagtgggacaaagagatcagcaactacacccagatcatctacggcctgctggaagagagccagaaccagcaagagaaaaacgagcaggacctgctggccctggacctgcctagcaccggaggatgataaggatcc

SEQ ID NO: 99 - protein, 852 aa, Artificial Sequence
  Description of Artificial Sequence Synthetic polypeptide
MRVRGIQRNCQHLWRWGTLILGMLMICSAAENLWVTVYYGVPVWKEANTTLFCASDAKAYDTEVHNVWATHACVPTDPNPQEIVLENVTENFNMWKNNMVEQMHEDIISLWDQSLKPCVKLTPLCVTLNCTNVNVTNTTNNTEEKGEIKNCSFNITTEIRDKKQKVYALFYRLDVVPIDDNNNNSSNYRLINCNTSAITQACPKVSFEPIPIHYCAPAGFAILKCNDKKFNGTGPCKNVSTVQCTHGIKPVVSTQLLLNGSLAEEEIIIRSENITNNAKTIIVQLNESVEINCTRPNNNTRKSIRIGPGQAFYATGDIIGDIRQAHCNISGTKWNKTLQQVAKKLREHFNNKTIIFKPSSGGDLEITTHSFNCRGEFFYCNTSGLFNSTWIGNGTKNNNNTNDTITLPCRIKQIINMWQGVGQAMYAPPIEGKITCKSNITGLLLTRDGGNNNTNETEIFRPGGGDMRDNWRSELYKYKVVKIEPLGVAPTKAKRRVVEREKRAVGIGAVFLGFLGAAGSTMGAASITLTVQARQLLSGIVQQQSNLLRAIEAQQHLLQLTVWGIKQLQARVLAVERYLKDQQLLGIWGCSGKLICTTTVPWNSSWSNKSQDEIWDNMTWMEWEREINNYTDIIYSLIEESQNQQEKNEQELLALDKWASLWNWFDITNWLWYIKIFIMIVGGLIGLRIVFAVLSIVNRVRQGYSPLSFQTLIPNPRGPDRPEGIEEEGGEQDRDRSIRLVNGFLALAWDDLRSLCLFSYHRLRDFILIAARTVELLGRKGLRRGWEALKYLWNLLQYWGQELKNSAISLLDTTAIAVAEGTDRVIEVVQRACRAILNIPRRIRQGLERALL

SEQ ID NO: 100 - protein, 5 aa, Artificial Sequence
  Description of Artificial Sequence Synthetic peptide
GGGGG

SEQ ID NO: 101 - protein, 6 aa, Artificial Sequence
  Description of Artificial Sequence Synthetic peptide
  MOD_RES (3)..(3): Any amino acid
LPXTGG

SEQ ID NO: 102 - protein, 2 aa, Artificial Sequence
  Description of Artificial Sequence Synthetic peptide
CK

(19) United States
(12) Patent Application Publication
(10) Pub. No.: US 2021/0009640 A1
(43) Pub. Date: Jan. 14, 2021

(54) COMPOSITIONS COMPRISING HIV ENVELOPES TO INDUCE HIV-1 ANTIBODIES

(71) Applicant: Duke University, Durham, NC (US)

(72) Inventor: Kevin O. SAUNDERS, Durham, NC (US)

(21) Appl. No.: 16/977,408

(22) PCT Filed: Mar. 1, 2019
(86) PCT No.: PCT/US2019/020436

§ 371 Date: Sep. 1, 2020

Related U.S. Application Data
(63) Continuation of application No. PCT/US18/20788, filed on Mar. 2, 2018.
(60) Provisional application No. 62/739,701, filed on Oct. 1, 2018.
(60) Provisional application No. 62/748,292, filed on Oct. 19, 2018.

(51) Int. Cl. C07K 14/16; A61K 38/16; C07K 17/00; A61K 47/69

(52) U.S. Cl. CPC C07K 14/16; A61K 47/6929; C07K 17/00; A61K 38/162

(57) ABSTRACT

The invention is directed to modified HIV-1 envelopes, compositions comprising these modified envelopes and methods of using these modified HIV-1 envelopes to induce immune responses.

This application claims the benefit and priority to International PCT application PCT/US2018/020788 filed Mar. 2, 2018, U.S. application Ser. No. 62/739,701 filed Oct. 1, 2018 and U.S. application Ser. No. 62/748,292 filed Oct. 19, 2018, the entire contents of each application are herein incorporated by reference.

This invention was made with government support under NIAID Research Grant (RO1AI120801). The government has certain rights in the invention.

### TECHNICAL FIELD

The present invention relates in general, to a composition suitable for use in inducing anti-HIV-1 antibodies, and, in particular, to immunogenic compositions comprising envelope proteins and nucleic acids to induce cross-reactive neutralizing antibodies and increase their breadth of coverage. The invention also relates to methods of inducing such broadly neutralizing anti-HIV-1 antibodies using such compositions.

### BACKGROUND

The development of a safe and effective HIV-1 vaccine is one of the highest priorities of the scientific community working on the HIV-1 epidemic. While anti-retroviral treatment (ART) has dramatically prolonged the lives of HIV-1 infected patients, ART is not routinely available in developing countries.

### SUMMARY OF THE INVENTION

In certain embodiments, the invention provides compositions and methods for induction of immune response, for example cross-reactive (broadly) neutralizing (bn) Ab induction. In certain embodiments, the methods use compositions comprising HIV-1 immunogens designed to bind to precursors, and/or UCAs of different HIV-1 bnAbs. In certain embodiments, these are UCAs of V1V2 glycan and V3 glycan antibodies.

In certain aspects, the invention provides compositions comprising a selection of HIV-1 envelopes and/or nucleic acids encoding these envelopes as described herein for example but not limited to selections as described herein. Without limitations, these selected combinations comprise envelopes which provide representation of the sequence (genetic) and antigenic diversity of the HIV-1 envelope variants which lead to the induction of V1V2 glycan and V3 glycan antibody lineages.

In certain embodiments the invention provides a recombinant CON-S HIV-1 envelope comprising substitutions at positions N138 and N141 (HXB2 numbering and FIG. 16) so that the envelope lacks glycosylation at these positions, and in some embodiments is glycosylated at N301 (HXB2 numbering and FIG. 16) and N332 (HXB2 numbering and FIG. 16). In certain embodiments the invention provides a recombinant CON-S HIV-1 envelope comprising substitutions at positions N130, N135, N138, and N141 (HXB2 numbering and FIG. 16) so that the envelope lacks glycosylation at these positions, and in some embodiments is glycosylated at N301 (HXB2 numbering and FIG. 16) and N332 (HXB2 numbering and FIG. 16).

In certain aspects, the invention provides a recombinant CON-S HIV-1 envelope comprising a V1 region, wherein the envelope lacks glycosylation at position N138 and N141 (HXB2 numbering and FIG. 16) and in some embodiments is glycosylated at N301 (HXB2 numbering and FIG. 16) and N332 (HXB2 numbering and FIG. 16).

In certain aspects, the invention provides a recombinant CON-S HIV-1 envelope comprising a V1 region, wherein the envelope lacks glycosylation at position N130, N135, N138, and N141 (HXB2 numbering and FIG. 16) and in some embodiments is glycosylated at N301 (HXB2 numbering) and N332 (HXB2 numbering and FIG. 16). The recombinant CON-S HIV-1 envelope further lacking glycosylation at position N138 and N141 (HXB2 numbering and FIG. 16) and in some embodiments is glycosylated at N301 (HXB2 numbering and FIG. 16) and N332 (HXB2 numbering and FIG. 16).

In certain embodiments, the CON-S HIV-1 envelope comprises the following substitutions at positions N130D, N135K, N138S, and N141S, and in some embodiments is glycosylated at N301 (HXB2 numbering) and N332 (HXB2 numbering). In certain embodiments, the CON-S HIV-1 envelope comprises the following substitutions at positions N138S and N141S, and in some embodiments is glycosylated at N301 (HXB2 numbering) and N332 (HXB2 numbering). Any other suitable amino acid substitution (X) is contemplated at these positions. In some embodiments, substitutions are made such that the structure and activity of CON-S is not substantially affected other than the glycosylation at the modified positions. In some embodiments, the substitutions could be selected from amino acids naturally occurring at these positions, wherein these positions are not glycosylated.

In certain aspects, the invention provides a recombinant CON-S HIV-1 envelope comprising a 17aa-long V1 region, wherein the envelope lacks glycosylation at positions N138 and N141 (HXB2 numbering) and in some embodiments is glycosylated at N301 (HXB2 numbering) and N332 (HXB2 numbering).

In certain aspects, the invention provides a recombinant CON-S HIV-1 envelope comprising a 17aa-long V1 region, wherein the envelope lacks glycosylation at positions N130, N135, N138, and N141 (HXB2 numbering) and in some embodiments is glycosylated at N301 (HXB2 numbering) and N332 (HXB2 numbering).

In certain embodiments, the recombinant envelope binds to precursors, and/or UCAs of different HIV-1 bnAbs. In certain embodiments, these are UCAs of V1V2 glycan and V3 glycan antibodies.

In certain embodiments, the envelope comprises all consecutive amino acids after the signal peptide of CON-Schim.6R.DS.SOSIP.664_OPT_N138X_N141X (Table 3).

In certain embodiments, the envelope comprises all consecutive amino acids after the signal peptide of CON-Schim.6R.DS.SOSIP.664_OPT_N138S_N141S (Table 3, FIG. 10).

In certain embodiments, the envelope comprises all consecutive amino acids after the signal peptide of CON-Schim.6R.DS.SOSIP.664_OPT_N130X_N135X_N138X_N141X_ (Table 3).

In certain embodiments, the envelope comprises all consecutive amino acids after the signal peptide of CON-Schim.6R.DS.SOSIP.664_OPT_N130D_N135K_N138S_N141S_ (Table 3, FIG. 10).

In certain embodiments, the envelope comprises all consecutive amino acids after the signal peptide of CON-Schim.6R.DS.SOSIP.664_OPT_N130X_N135X_N138X_N141X_ferritin (Table 3).

In certain embodiments, the envelope comprises all consecutive amino acids after the signal peptide of CON-Schim.6R.DS.SOSIP.664_OPT_N130D_N135K_N138S_N141S_ferritin (Table 3, FIG. 10, FIG. 11B).

In certain embodiments, the envelope comprises all consecutive amino acids after the signal peptide of CON-Schim.6R.DS.SOSIP.664_OPT_D1305V1 (Table 1, FIG. 13).

In certain aspects, the invention provides a recombinant CON-S envelope wherein the envelope is a protomer comprised in a stable trimer.

In certain embodiments, the envelope is any one of the envelopes of Table 1, 2 or 3. A skilled artisan appreciates that when recombinantly expressed the envelope proteins do not include a signal sequence. The CON-S recombinant of any of the embodiments, wherein the envelope is designed so that it forms a multimer, wherein the multimer is a trimer, or in some embodiment, the multimer comprises several trimers, e.g. but not limited to trimers arrayed in a ferritin nanoparticle.

In certain embodiments, the recombinant Con-S envelope is Man9GlcNAc-enriched. In some embodiments the envelopes are recombinantly produced under kif treatment.

In certain embodiments, the envelope comprises additional mutations stabilizing the trimer. In certain embodiments these including but are not limited to SOSIP mutations. In certain embodiments mutations are selected from sets F1-F14, VT1-VT8 mutations described herein, or any combination or subcombination within a set.

In certain aspects, the invention provides a composition comprising any one the recombinant CON-S envelopes and a carrier.

In certain aspects, the invention provides a nucleic acid encoding any one of the recombinant envelopes of the invention. In certain aspects, the invention provides a composition comprising a nucleic acid encoding the recombinant proteins of the invention and a carrier. In certain embodiments, the nucleic acid is a modified mRNA.

In certain aspects, the invention provides composition comprising the recombinant CON-S envelope of the invention, wherein the recombinant CON-S envelope is multimerized and wherein optionally in some embodiments the envelope is comprised in a nanoparticle.

In certain embodiments, the recombinant CON-S envelope is comprised in a nanoparticle that is a ferritin nanoparticle.

In certain aspects, the invention provides methods of inducing an immune response in a subject comprising administering an immunogenic composition comprising the recombinant CON-S envelopes of the invention or a composition comprising the inventive recombinant CON-S envelopes. In certain embodiments, the methods comprise administering a series of immunogenic compositions, a non-limiting embodiment shown in FIG. 3A.

In certain embodiments of the methods, the immunogenic composition comprises a first immunogen comprising all consecutive amino acids after the signal peptide of CON-Schim.6R.DS.SOSIP.664_OPT_N130D_N135K_N138S_N141S_ferritin (Table 3, FIG. 10, FIG. 11B), and wherein the immunogen is optionally Man9GlcNAc-enriched, optionally multimerized in a nanoparticle, wherein the nanoparticle is a ferritin nanoparticle.

In certain embodiments, the methods further comprise administering a second immunogenic composition comprising a second immunogen comprising all consecutive amino acids after the signal peptide of CON-Schim.6R.DS.SOSIP.664_OPT_N130D_N135K_N138S_N141S_ (Table 3, FIG. 10, FIG. 11B), and wherein the immunogen is optionally multimerized in a trimer.

In certain embodiments, the methods further comprise administering a third immunogenic composition comprising a third immunogen comprising all consecutive amino acids after the signal peptide of CON-Schim.6R.DS.SOSIP.664_OPT_N138S_N141S (Table 3, FIG. 10), wherein the immunogen is optionally Man9GlcNAc-enriched and optionally multimerized in a trimer.

In certain embodiments, the methods further comprise administering a fourth immunogenic composition comprising a fourth immunogen comprising all consecutive amino acids after the signal peptide of CON-Schim.6R.DS.SOSIP.664_OPT_N138S_N141S (Table 3, FIG. 10), wherein the immunogen is optionally multimerized in a trimer.

In certain embodiments, the methods further comprise administering a fifth immunogenic composition comprising a fifth immunogen comprising all consecutive amino acids after the signal peptide of CON-Schim.6R.DS.SOSIP.664_OPT_ (Table 3, FIG. 10), wherein the immunogen is optionally Man9GlcNAc-enriched and optionally multimerized in a trimer.

In certain embodiments, the methods further comprise comprising administering a sixth immunogenic composition comprising a sixth immunogen comprising all consecutive amino acids after the signal peptide of CON-Schim.6R.DS.SOSIP.664_OPT_ (Table 3, FIG. 10), wherein the immunogen is optionally multimerized in a trimer.

In certain embodiments, the methods further comprise administering an adjuvant.

In certain embodiments of the methods, the composition is administered as a prime and/or a boost. In certain embodiments, the composition comprises nanoparticles.

In certain aspects, the invention provides a recombinant HIV-1 envelope comprising 17aa V1 region, lacks glycosylation at position N133 and N138 (HXB2 numbering), includes glycosylation at N301 (HXB2 numbering) and N332 (HXB2 numbering), and optionally further comprises the “GDIR” motif

In certain embodiments, the recombinant envelope binds to precursors, and/or UCAs of different HIV-1 bnAb s. In certain embodiments, these are UCAs of V1V2 glycan and V3 glycan antibodies. In certain embodiments, the envelope is 19CV3. In certain embodiments, the envelope is not 10.17 DT variant described previously in WO/2018161049.

In certain embodiments, the envelope is a protomer which could be comprised in a stable trimer. In certain embodiments, the envelope comprises additional mutations stabilizing the trimer. In certain embodiments these including but are not limited to SOSIP mutations. In certain embodiments, mutations are selected from sets F1-F14, VT1-VT8 mutations described herein, or any combination or subcombination within a set.

In certain embodiments, recombinant HIV-1 envelopes are shown in FIGS. 10-16. A nucleic acid encoding any of the recombinant envelopes. In certain aspects, the invention provides a composition comprising any one of the inventive envelopes or nucleic acid sequences encoding the same.

Provides are compositions comprising a nanoparticle which comprises any one of the envelopes of the invention. The composition of any of the embodiments, wherein the nanoparticle is ferritin self assembling nanoparticle.

In certain aspects, the invention provides methods of inducing an immune response in a subject comprising administering an immunogenic composition comprising any one of the stabilized envelopes of the invention. In certain embodiments, the composition is administered as a prime and/or a boost. In certain embodiments, the composition comprises nanoparticles.

In certain aspects, the invention provides compositions comprising a plurality of nanoparticles comprising a plurality of the envelopes/trimers of the invention, and a carrier. In non-limiting embodiments, the envelopes/trimers of the invention are multimeric when comprised in a nano-particle. The nanoparticle size is suitable for delivery and purification. In non-liming embodiments, the nanoparticles are ferritin-based nano-particles.

In certain aspects, the invention provides a ConS envelope where four glycans are removed: N130, N135, N138 and N141. In some embodiments these glycans are removed by the following amino acid changes N130D, N135K, N138S, and N141S. Any ConS envelope comprising any one of these glycan site modifications, or combination thereof is contemplated.

In certain aspects the invention provides a ConS envelope where two glycans are removed: N138 and N141. In some embodiments these glycans are removed by the following amino acid changes N138S and N141S. Any ConS envelope comprising these glycan site modifications is contemplated.

In certain embodiments the inventive envelopes are produced recombinantly in the presence of kifunensine. Recombinant envelopes grown in kif treated cells are Man9GlcNAc2 enriched.

### BRIEF DESCRIPTION OF THE DRAWINGS

The patent or application file contains at least one drawing executed in color. To conform to the requirements for PCT patent applications, many of the figures presented herein are black and white representations of images originally created in color.

FIGS. 1A-1B show that Kif treatment improves V3-glycan bnAb neutralization. (A) Structural model of HIV-1 Env depicting bnAb epitopes in different colors. (B) Neutralization of untreated and kif-treated JRFL by the V3-glycan bnAb lineage DH270.

FIGS. 2A-2E show that V3-glycan bnAbs and precursor antibodies bind optimized CON-S SOSIP trimers. (A-B) 2D-class average of negative stain EM images of (A) kif-treated SOSIP nanoparticles and (B) free trimers. (C) Kif-treated CON-S nanoparticles can bind to unmutated common ancestors (UCAs) of V1V2 and V3-glycan bnAbs. (D) Kif-treated CON-S nanoparticle binds with a higher magnitude to affinity matured DH270 than DH270 UCA3. (E) Kif treatment and nanoparticle presentation enhances DH270 binding to CON-S.

FIGS. 3A-3C show rapid induction of CON-S binding antibodies in immunized macaques. (A) Vaccination Regimen. Sequential vaccine regimen aimed at eliciting V3-glycan antibodies. Immunization of rhesus macaques to determine immunogenicity of CON-S SOSIP nanoparticles. (B) 2D-class average negative stain EM images of HIV-1 Env immunogens and antigenicity profile. (C) Serum IgG binding titers to CON-S and CH848 envelopes and ferritin. Arrows indicate immunization time points.

FIG. 4 shows that vaccinated rhesus macaque serum blocks the binding of V3-glycan bnAb DH270 and glycan bnAb 2G12 to Env. Serum blocking was measured in a competition ELISA. Greater than 20% is considered positive. Blocking arose quicker on Envs where V1 glycans were removed.

FIG. 5 shows vaccination induced N301 glycan-dependent autologous tier 2 neutralization. Serum neutralization of autologous tier 2 viruses arose after three immunizations in all 4 rhesus macaques. A N301A mutation decreased neutralization in 2 of 4 macaques. Kifunensine-dependent serum neutralization of JRFL was also elicited.

FIG. 6 shows one embodiment of Con-S vaccine design.

FIG. 7 shows Preliminary ConS glycans.

FIG. 8 shows CON-S vs BG505 SOSIPs glycans.

FIGS. 9A-9B show Disulfide Bond Topology: CON-Schim.6R.DS.SOSIP.664_avi _OPT_N130D_N135K_N138S_N141S (mutant). FIG. 9A shows canonical Disulfide Bond Topology. FIG. 9B shows alternative Disulfide Bond Topology.

FIG. 10 shows non-limiting embodiments of sequences.

FIG. 11A shows a non-limiting embodiment of a Con-S sequence SOSIP design with a modified V1 loop. In one embodiment, the V1 loop is from a naturally occurring envelope CH848.3.D1305.10.19. Bolded is the position of the V1 loop; some amino acids are shared, i.e. are the same between the ConS V1 loop and 10.19 V1 loop, but the ones that are different are modified in ConS. In some embodiments of the invention, the 10.19 V1 loop can replace the V1 loop in any of the ConS sequences and designs.

FIG. 11B shows a non-limiting embodiment of an HIV-1 envelope comprising a ferritin sequence for multimerization. This sequence comprises as annotated: a cloning site at the beginning of the sequence, indicated by the italicized sequence in this figure, a signal peptide, indicated by the underlined position, and one embodiment of a liker between the envelope sequences and the ferritin protein, indicated by the bolded amino acids.

FIG. 12 shows non-limiting embodiments of sequences.

FIG. 13 shows non-limiting embodiments of sequences.

FIG. 14 shows non-limiting embodiments of sequences.

FIG. 15 shows the sequence the sortase A tagged SOSIP trimer HV1301580_C_SORTA;CH848.3.D1305.10.19_D949V3.DS.SOSIP_C_SORTA. The sequence is of sortase A tagged SOSIP trimer. The Sortase A tag is LPSTGG which is modified from LPSTG because an additional Gly residue helps accelerate the reaction rate.

FIG. 16 shows sequences of CON-S gp160 envelope with four N glycosylation sites (N130, N135, N138 and N141 bolded and underlined).

FIG. 17A-17B shows schematic of an HIV envelope SOSIP ferritin nanoparticles by sortase-A conjugation. FIG. 17A. Diagram of one non-limiting embodiment of HIV-1 envelope SOSIP trimer showing the orientation of sortase A linkage to ferritin. The sortase linker in this embodiment is LPSTGG. FIG. 17B. A model of an HIV-1 env SOSIP ferritin particle with 8 Env trimers displayed, based on ferritin and SOSIP trimer crystal structures.

FIG. 18A-C Ca2+ flux experiments. These data show that Con-S delta V1 glycans triggers DH270 IA4 but JRFL degly (“Q3”) does not. FIG. 18A. Experiment Name: CaF020; Protein: CON-Sgp140CFI.avi/293F/Trimer; Concentration Assayed: 0.100 nMolar of protein tetramer; Data presented as % of max anti-IgM Fab(2) 50 ug/mL. FIG. 18B. Experiment Name: CaF020; Protein: JRFLgp140CF.avi V1 3Q/293F/Trimer; Concentration Assayed: 0.100 nMolar of protein tetramer; Data presented as % of max anti-IgM Fab(2) 50 ug/mL. FIG. 18C. Experiment Name: CaF020; Protein: CON-Sgp140CFI.avi/293F/Trimer; Concentration Assayed: 0.100 nMolar of protein tetramer; Data presented as % of max anti-IgM Fab(2) 50 ug/mL.

FIGS. 19A-19C show expression of sequential CON-S stabilized SOSIP trimers with V1 glycans present (A) or removed, (B) two glycans N138S and N141S are removed and (C) four glycans N130D, N135K, N138S, and N141S are removed. Top panel shows size exclusion chromatography and bottom panel shows negative stain EM. Expression of stabilized CON-S SOSIP gp140 trimers with serially deleted V1 glycosylation sites. Size exclusion chromatography shows most of the protein purified with PGT145 affinity chromatography is trimeric Env. Trimeric envelope was visualized by negative stain electron microscopy and 2-dimensional class averaging.

FIG. 20 shows Glycosylation profile of the stabilized CON-S SOSIP. The data shows that CON-S SOSIPs are glycosylated with only high mannose at the N332 glycan bnAb epitope. The same glycosylation profile was obtained when the four V1 glycans were removed. The glycosylation profile of CON-S SOSIP with (CON-S gp140 chim. 6R.SOSIP.664.avi) and without V1 glycans at N130, N135, N138, N141 (CON-S gp140 chim. 6R.SOSIP.664.avi_opt mutant). Mass spectrometry shows that the CON-S SOSIP gp140 with N130D, N135K, N138S, and N141S mutations lacks any glycans at N130, N135, N138, N141. The percentage of high mannose at neutralizing antibody contact sites 295, 301, 332, 156, and 611 does not change with removal of the glycans.

FIGS. 21A-21B show CON-S Ferritin nanoparticles by SOSIP-ferritin fusion proteins. (A) shows SOSIP-Ferritin, (B) shows 2D class average. Fusion of CON-S SOSIP N130D, N135K, N138S, and N141S to H. pylori ferritin to create nanoparticles. Negative stain electron microscopy shows nanoparticle formation of CON-S SOSIP

FIG. 22 shows Glycan-modified CON-S binds to V1V2-glycan and V3-glycan bnAb UCAs. The data show V1V2 glycan and V3-glycan bnAb UCA antigenicity. CON-S SOSIP N130D, N135K, N138S, and N141S ferritin nanoparticle is antigenic for V3-glycan bnAb unmutated common ancestors (UCAs) antibodies and V1V2-glycan bnAb precursor CH103 UCA antibody. V3-glycan bnAbs are BF520, BG18, and DH270. DH272 is a V3-glycan antibody that neutralizes only autologous viruses. CHO1 is a V1V2-glycan bnAb. Binding was measured by biolayer interferometry with the nanoparticle in solution and each antibody immobilized on a sensor tip. The red vertical line indicates the end of the association phase.

FIGS. 23A-23B show that Man9GlcNAc2 enrichment on CON-S ferritin nanoparticles augments V3-glycan bnAb binding. (A) shows kif treated CON-S SOSIP ferritin nanoparticle. (B) shows DH270 V3 glycan antibody binding to CON-S. Kifunensine treatment enhances V3-glycan bnAb binding to CON-S SOSIP N130D, N135K, N138S, and N141S ferritin nanoparticle. Binding of V3-glycan bnAb DH270 to CON-S SOSIP N130D, N135K, N138S, and N141S was compared to binding to CON-S SOSIP N130D, N135K, N138S, and N141S ferritin nanoparticle. To enrich the glycans on the CON-S SOSIP N130D, N135K, N138S, and N141S ferritin nanoparticle for Man9GlcNAc2 or Man8GlcNAc2 the protein was expressed in cells treated with the glycosidase inhibitor kifunensine.

FIGS. 24A-24B show that a single ferritin nanoparticle of CON-S SOSIP lacking four glycans immunization elicited gradually increasing serum IgG over 6 weeks. Numbers on the x-axis show the study week. Arrows indicate immunization.

FIG. 25 shows serum IgG binding to CON-S after trimer boost (II in FIG. 3A) was blocked by V1 glycans on the CON-S SOSIP.

FIGS. 26A-26B show that NHP serum antibodies block V3-glycan mAb DH270 (A) and glycan mAb 2G12 (B) binding to CON-S SOSIP. Numbers on the x-axis show the study week. Arrows indicate immunization. Plasma antibodies from CON-S SOSIP vaccinated macaques blocks V3-glycan bnAb DH270 and gp120 glycan bnAb 2G12 binding to CON-S SOSIP gp140.

FIG. 27 shows that CON-S SOSIP vaccination induced autologous tier 2 neutralization. Numbers on the x-axis show the study week. Arrows indicate immunization.

FIG. 28 shows that autologous tier 2 neutralization is not dependent on the N362 glycan hole. The glycan shield of CON-S is intact except at N362.

FIGS. 29A-29B show that two of the NHPs in the study in Example 2 generated N301-dependent autologous tier 2 neutralizing antibodies (compare 29A and 29B). The figure shows CON-S neutralization (heterogenous glycans). Numbers on the x-axis show the study week. Arrows indicate immunization. Immunization induces autologous tier 2 neutralizing antibodies in two macaques that target the N301 glycan in the V3-glycan epitope on Env. Neutralization titer was measured in the TZM-bl assay as serum dilution that inhibits 50% of virus replication (ID50). Solid lines are the neutralization titers for wildtype CON-S. Dashed lines are neutralization titers for CON-S with the asparagine301 (N301) mutated to alanine to disrupt the glycosylation sequence. Arrows on the x-axis indicate immunization timepoints.

FIGS. 30A-30F show BLI data for 442EML(b) CON_Schim.6R.DS.SOSIP.664_OPT_N130D_N135K_N138S_ (kif treated envelope)—(50 ug/mL). Vertical dotted lines indicate response at end of association. Panels show biolayer interferometry binding of a panel of HIV-1 antibodies to the CON-S envelope nanoparticle. The data shows that there is trimeric envelope present on the nanoparticle and that the envelope presents four bnAb epitopes. The cells producing the nanoparticle are treated with kifunensine to enrich for Man9GlcNac2 glycans during protein synthesis. The CON-S protein binds to trimer-specific antibody PGT145. It weakly binds to trimer-specific antibody PGT151 because the Env glycosylation has been restricted to Man9GlcNAc2 and PGT151 requires complex glycans. The envelope is not antigenic for the inferred precursor of the CH106 lineage (CH103 UCA) but can bind to somatically mutated broadly neutralizing antibodies against the CD4 binding site such as VRCO1 and CH106. The envelope is not antigenic for antibodies that recognize the CD4-induced conformation of Env (A32, 17B, and CH58). Antibodies against the conformational V3 glycan bnAb epitope bind to the envelope showing this envelope has a well-folded V3 loop and the prototypical HIV-1 envelope high mannose patch is present on the envelope. The binding of 19B and F39F shows that not all of the envelope is the closed conformation, which has the V3 loop inaccessible to 19B and F39F.

FIG. 31 shows a summary CON-Schim.6R.DS.SOSIP.664_OPT_N130D_N135K_N138S_N141S_ferritin/5uM-Kif/293F 442EML Binding Results. Panels show biolayer interferometry binding of a panel of HIV-1 antibodies to the CON-S envelope nanoparticle. The data shows that there is trimeric envelope present on the nanoparticle and that the envelope presents four bnAb epitopes. The CON-S protein binds to trimer-specific antibodies PGT145 and PGT151. The envelope is not antigenic for the inferred precursor of the CH106 lineage (CH103 UCA) but can bind to somatically mutated broadly neutralizing antibodies against the CD4 binding site such as VRC01 and CH106. The envelope is not antigenic for antibodies that recognize the CD4-induced conformation of Env (A32, 17B, and CH58). Antibodies against the conformational V3 glycan bnAb epitope bind to the envelope showing this envelope has a well-folded V3 loop and the protypical HIV-1 envelope high mannose patch is present on the envelope. The binding of 19B and F39F shows that not all of the envelope is the closed conformation, which has the V3 loop inaccessible to 19B and F39F.

FIGS. 32A-32F BLI data for 455EML CONSchim.6R.DS.SOSIP.664_OPT_N130D_N135K_N138S_N141 (kif untreated)—(50 ug/mL). Vertical dotted lines indicate response at end of association.

FIG. 33 shows a summary CON-Schim.6R.DS.SOSIP.664_OPT_N130D_N135K_N138S_N141S_ferritin/293F 455EML 17-July-2018 Binding Results.

FIG. 34 shows a summary of antigenic profile of CON-S envelopes with various V1 glycosylation sites removed.

### DETAILED DESCRIPTION OF THE INVENTION

The development of a safe, highly efficacious prophylactic HIV-1 vaccine is of paramount importance for the control and prevention of HIV-1 infection. A major goal of HIV-1 vaccine development is the induction of broadly neutralizing antibodies (bnAbs) (Immunol. Rev. 254: 225-244, 2013). BnAbs are protective in rhesus macaques against SHIV challenge, but as yet, are not induced by current vaccines.

The invention provides methods of using these pan bnAb envelope immunogens.

In certain aspect, the invention provides compositions for immunizations to induce lineages of broad neutralizing antibodies. In certain embodiments, there is some variance in the immunization regimen; in some embodiments, the selection of HIV-1 envelopes may be grouped in various combinations of primes and boosts, either as nucleic acids, proteins, or combinations thereof. In certain embodiments, the compositions are pharmaceutical compositions which are immunogenic. In certain embodiments, the compositions comprise amounts of envelopes which are therapeutic and/or immunogenic.

In one aspect the invention provides a composition for a prime boost immunization regimen comprising any one of the envelopes described herein, or any combination thereof wherein the envelope is a prime or boost immunogen. In certain embodiments, the composition for a prime boost immunization regimen comprises one or more envelopes described herein.

In certain embodiments, the compositions contemplate nucleic acid, as DNA and/or RNA, or proteins immunogens either alone or in any combination. In certain embodiments, the methods contemplate genetic, as DNA and/or RNA, immunization either alone or in combination with envelope protein(s).

mRNA

In some embodiments, the antigens are nucleic acids, including but not limited to mRNAs which could be modified and/or unmodified. See U.S. Pub 20180028645A1, U.S. Pub 20170369532, U.S. Pub 20090286852, U.S. Pub 20130111615, U.S. Pub 20130197068, U.S. Pub 20130261172, U.S. Pub 20150038558, U.S. Pub 20160032316, U.S. Pub 20170043037, U.S. Pub 20170327842, each content is incorporated by reference in its entirety. mRNAs delivered in LNP formulations have advantages over non-LNPs formulations. See U.S. Pub 20180028645A1.

In certain embodiments, the nucleic acid encoding an envelope is operably linked to a promoter inserted an expression vector. In certain aspects, the compositions comprise a suitable carrier. In certain aspects, the compositions comprise a suitable adjuvant.

In certain embodiments, the induced immune response includes induction of antibodies, including but not limited to autologous and/or cross-reactive (broadly) neutralizing antibodies against HIV-1 envelope. Various assays that analyze whether an immunogenic composition induces an immune response, and the type of antibodies induced are known in the art and are also described herein.

In certain aspects, the invention provides an expression vector comprising any of the nucleic acid sequences of the invention, wherein the nucleic acid is operably linked to a promoter. In certain aspects, the invention provides an expression vector comprising a nucleic acid sequence encoding any of the polypeptides of the invention, wherein the nucleic acid is operably linked to a promoter. In certain embodiments, the nucleic acids are codon optimized for expression in a mammalian cell, in vivo or in vitro. In certain aspects, the invention provides nucleic acids comprising any one of the nucleic acid sequences of invention. In certain aspects, the invention provides nucleic acids consisting essentially of any one of the nucleic acid sequences of invention. In certain aspects, the invention provides nucleic acids consisting of any one of the nucleic acid sequences of invention. In certain embodiments the nucleic acid of the invention, is operably linked to a promoter and is inserted in an expression vector. In certain aspects, the invention provides an immunogenic composition comprising the expression vector.

In certain aspects, the invention provides a composition comprising at least one of the nucleic acid sequences of the invention. In certain aspects, the invention provides a composition comprising any one of the nucleic acid sequences of invention. In certain aspects, the invention provides a composition comprising at least one nucleic acid sequence encoding any one of the polypeptides of the invention.

The envelope used in the compositions and methods of the invention can be a gp160, gp150, gp145, gp140, gp120, gp41, N-terminal deletion variants as described herein, cleavage resistant variants as described herein, or codon optimized sequences thereof In certain embodiments, the composition comprises envelopes as trimers. In certain embodiments, envelope proteins are multimerized, for example, trimers are attached to a particle such that multiple copies of the trimer are attached and the multimerized envelope is prepared and formulated for immunization in a human. In certain embodiments, the compositions comprise envelopes, including but not limited to trimers as particulate, high-density array on liposomes or other particles, for example but not limited to nanoparticles. In some embodiments, the trimers are in a well ordered, near native like or closed conformation. In some embodiments, the trimer compositions comprise a homogenous mix of native like trimers. In some embodiments, the trimer compositions comprise at least 85%, 90%, or 95% native like trimers.

In certain embodiments, the envelope is any of the forms of HIV-1 envelope. In certain embodiments, the envelope is gp120, gp140, gp145 (i.e. with a transmembrane), or gp150. In certain embodiments, gp140 designed to form a stable trimer. In certain embodiments envelope protomers from a trimer which is not a SOSIP timer. In certain embodiment, the trimer is a SOSIP based trimer wherein each protomer comprises additional modifications. In certain embodiments, envelope trimers are recombinantly produced. In certain embodiments, envelope trimers are purified from cellular recombinant fractions by antibody binding and reconstituted in lipid comprising formulations. See for example WO2015/127108 titled “Trimeric HIV-1 envelopes and uses thereof” and WO/2017151801 which content is herein incorporated by reference in its entirety. In certain embodiments, the envelopes of the invention are engineered and comprise non-naturally occurring modifications.

In certain embodiments, the envelope is in a liposome. In certain embodiments, the envelope comprises a transmembrane domain with a cytoplasmic tail embedded in a liposome. In certain embodiments, the nucleic acid comprises a nucleic acid sequence, which encodes a gp120, gp140, gp145, gp150, or gp160.

In certain embodiments, where the nucleic acids are operably linked to a promoter and inserted in a vector, the vector is any suitable vector. Non-limiting examples include, VSV, replicating rAdenovirus type 4, MVA, Chimp adenovirus vectors, pox vectors, and the like. In certain embodiments, the nucleic acids are administered in NanoTaxi block polymer nanospheres. In certain embodiments, the composition and methods comprise an adjuvant. Non-limiting examples include, 3M052, AS01 B, AS01 E, gla/SE, alum, Poly I poly C (poly IC), polylC/long chain (LC) TLR agonists, TLR7/8 and 9 agonists, or a combination of TLR7/8 and TLR9 agonists (see Moody et al. (2014) J. Virol. March 2014 vol. 88 no. 6 3329-3339), or any other adjuvant. Non-limiting examples of TLR7/8 agonist include TLR7/8 ligands, Gardiquimod, Imiquimod and R848 (resiquimod). A non-limiting embodiment of a combination of TLR7/8 and TLR9 agonist comprises R848 and oCpG in STS (see Moody et al. (2014) J. Virol. March 2014 vol. 88 no. 6 3329-3339).

In certain aspects the invention provides a cell comprising a nucleic acid encoding any one of the envelopes of the invention suitable for recombinant expression. In certain aspects, the invention provides a clonally derived population of cells encoding any one of the envelopes of the invention suitable for recombinant expression. In certain aspects, the invention provides a sable pool of cells encoding any one of the envelopes of the invention suitable for recombinant expression.

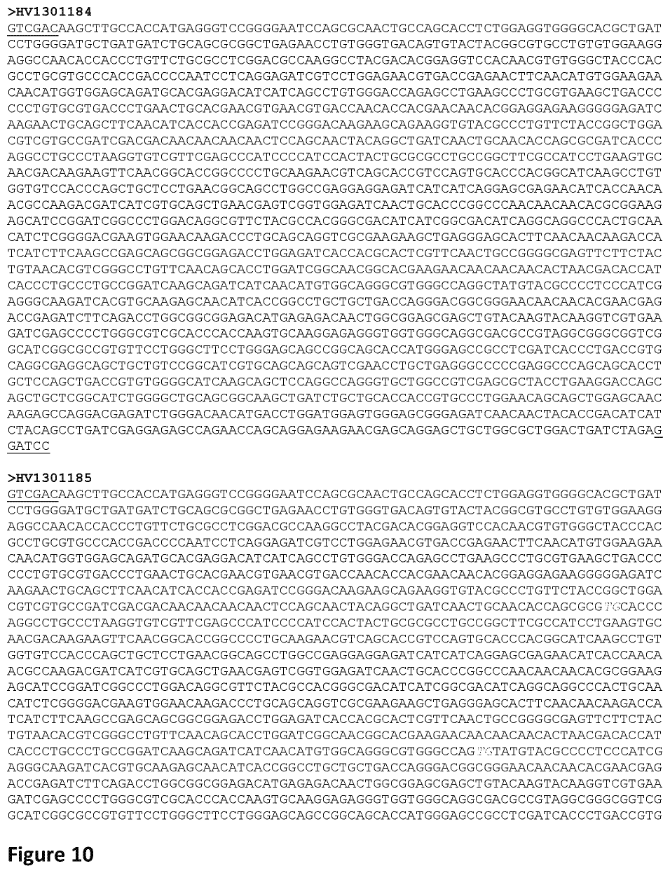

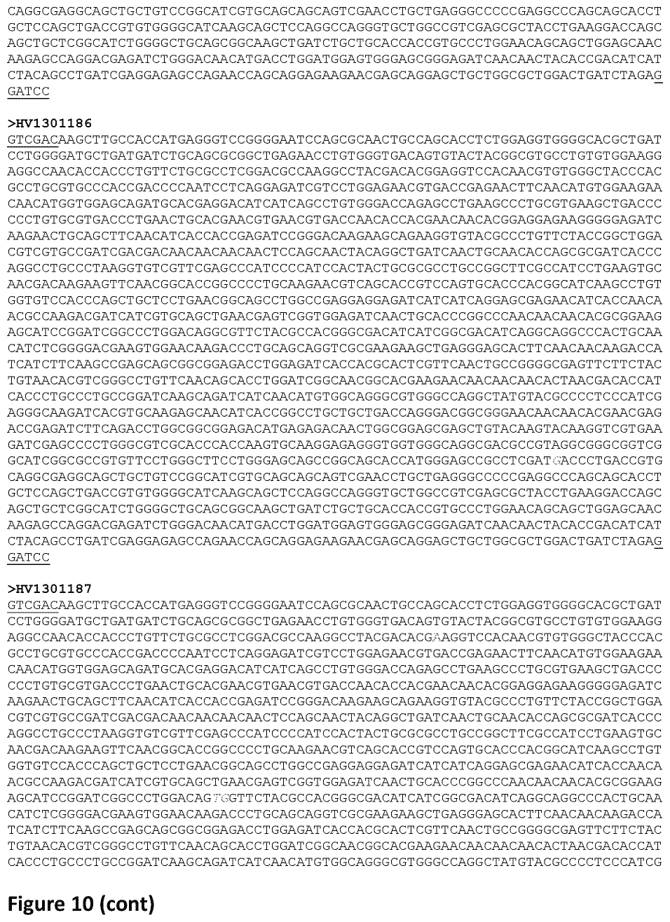

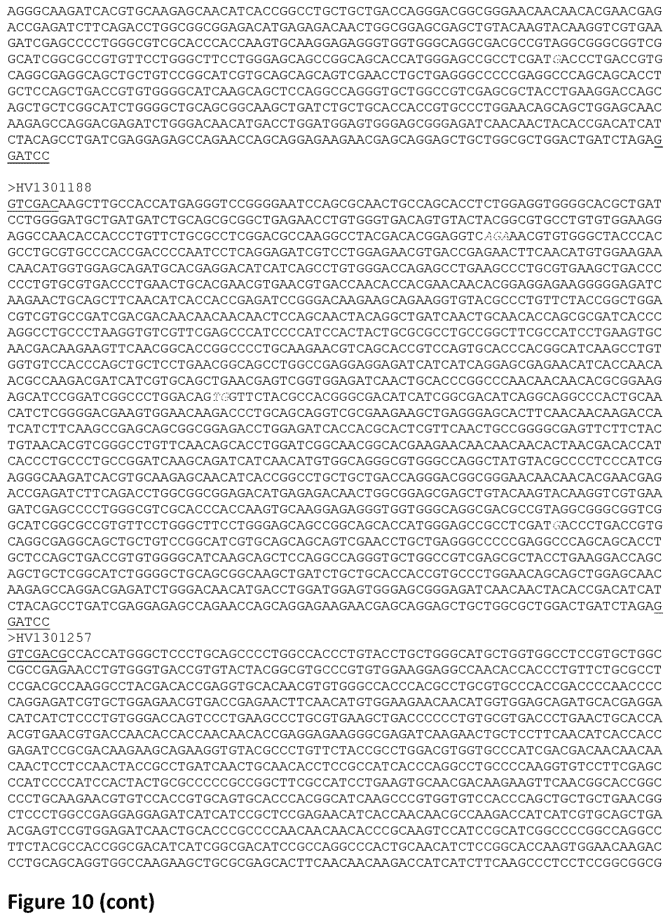

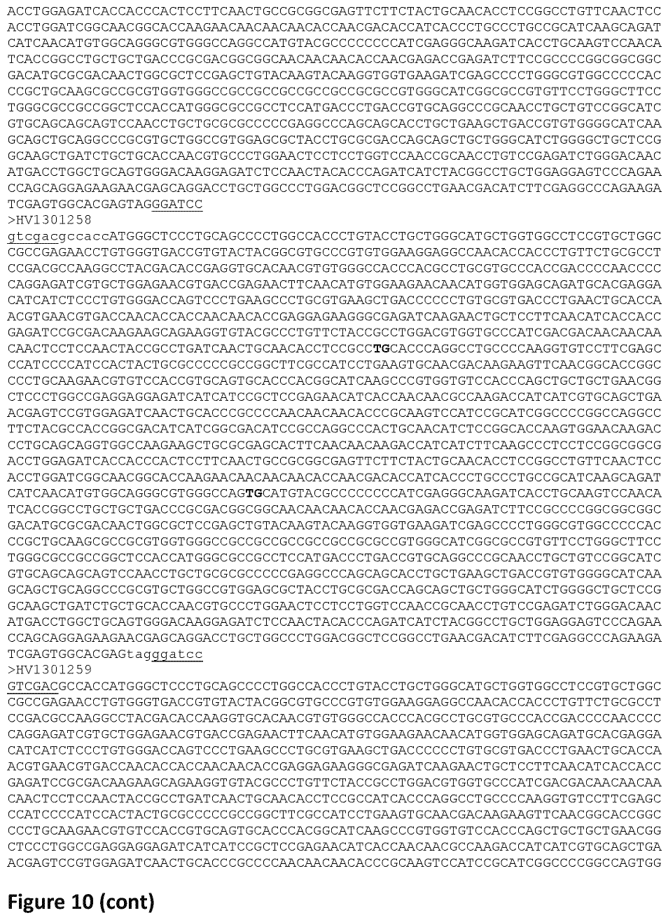

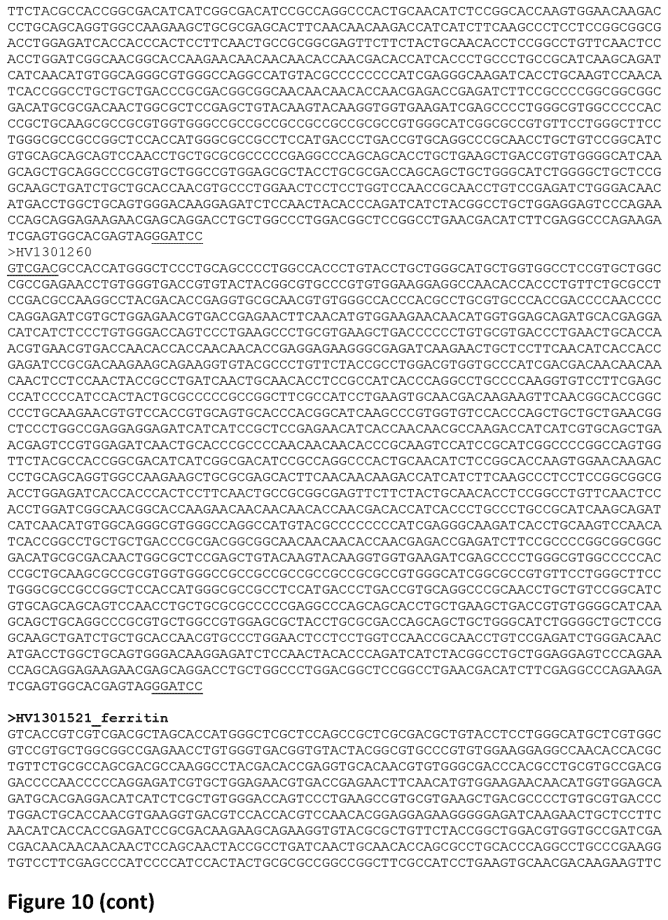

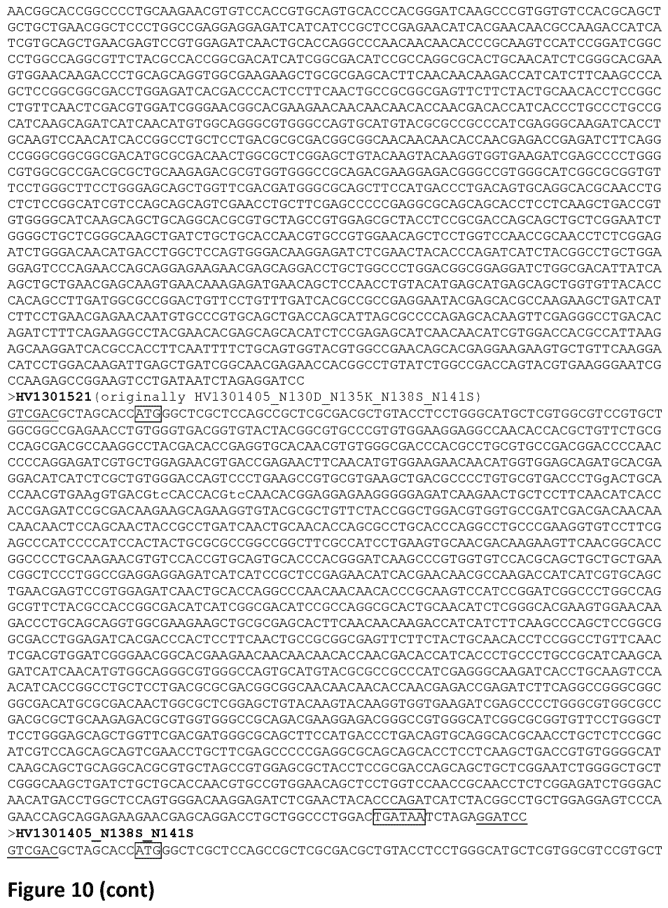

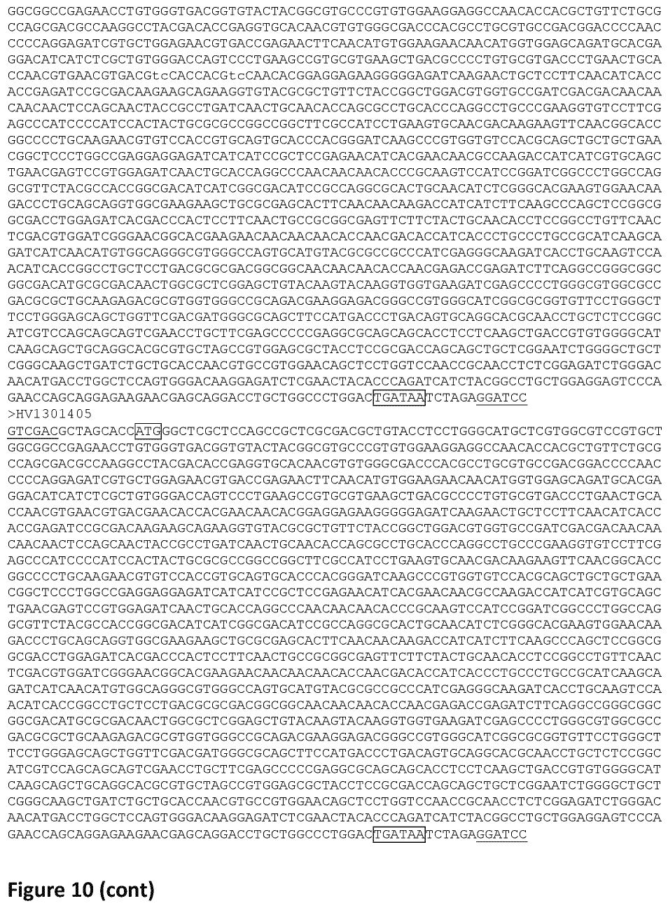

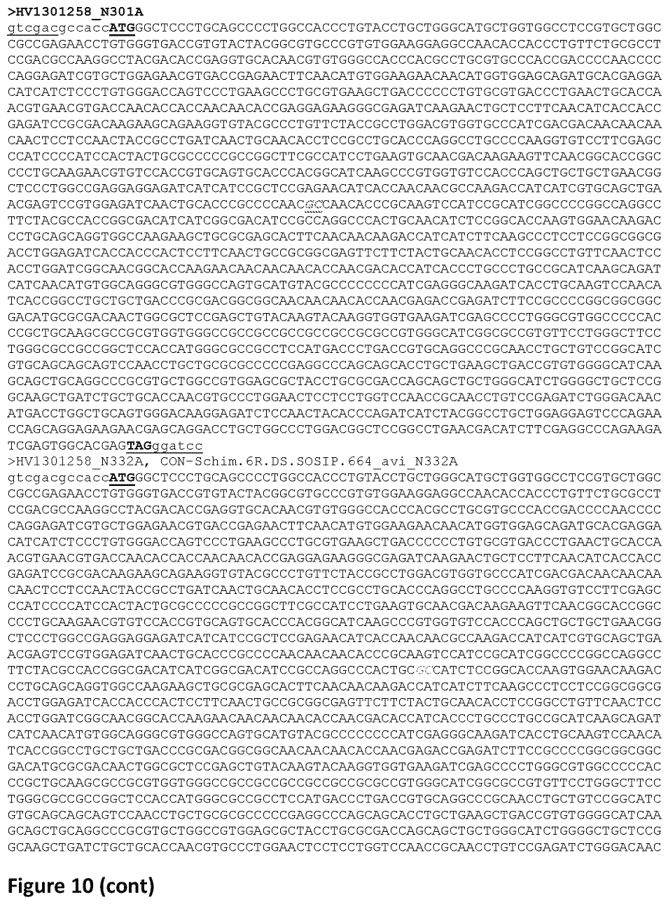

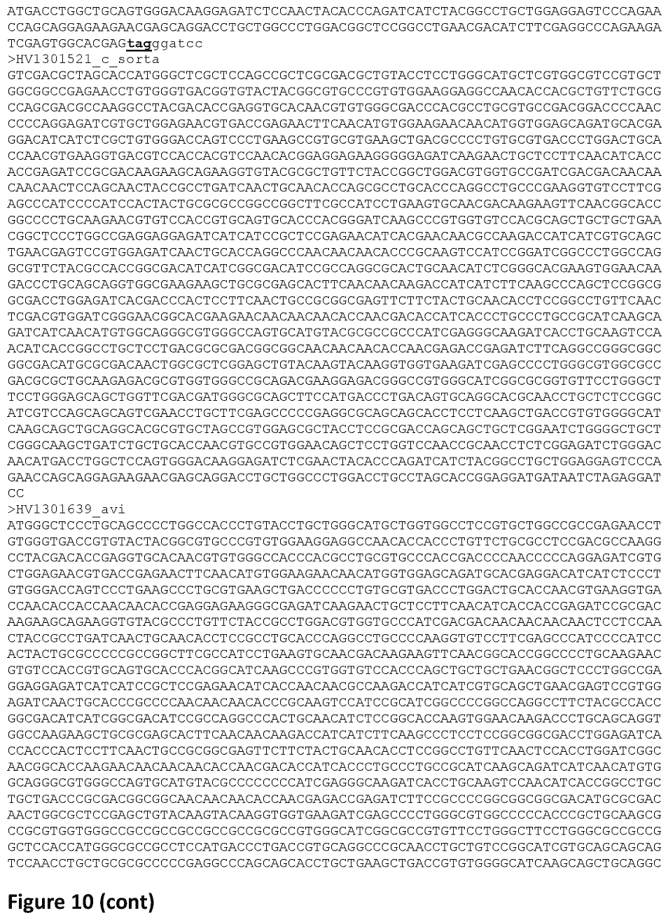

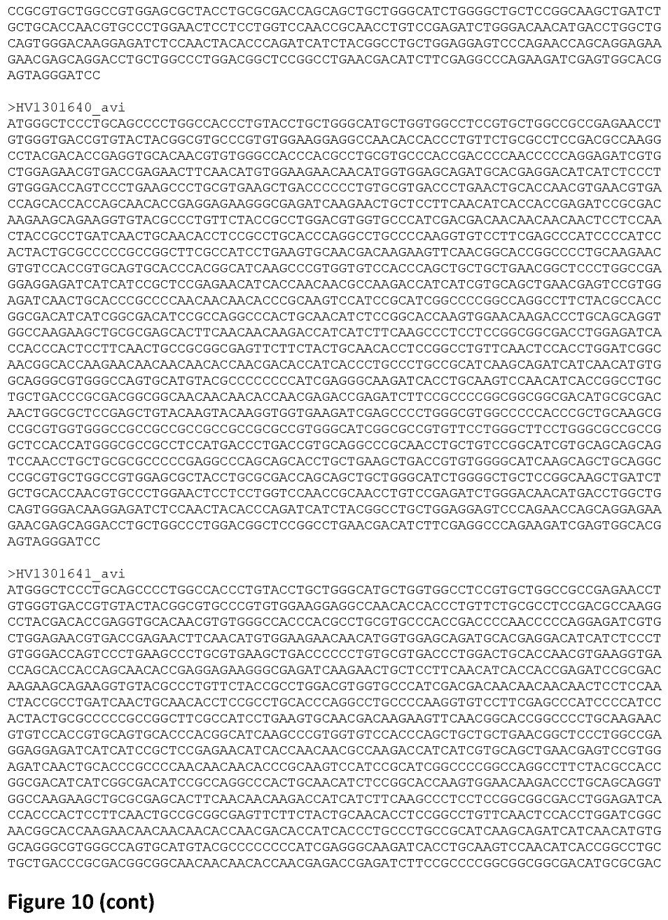

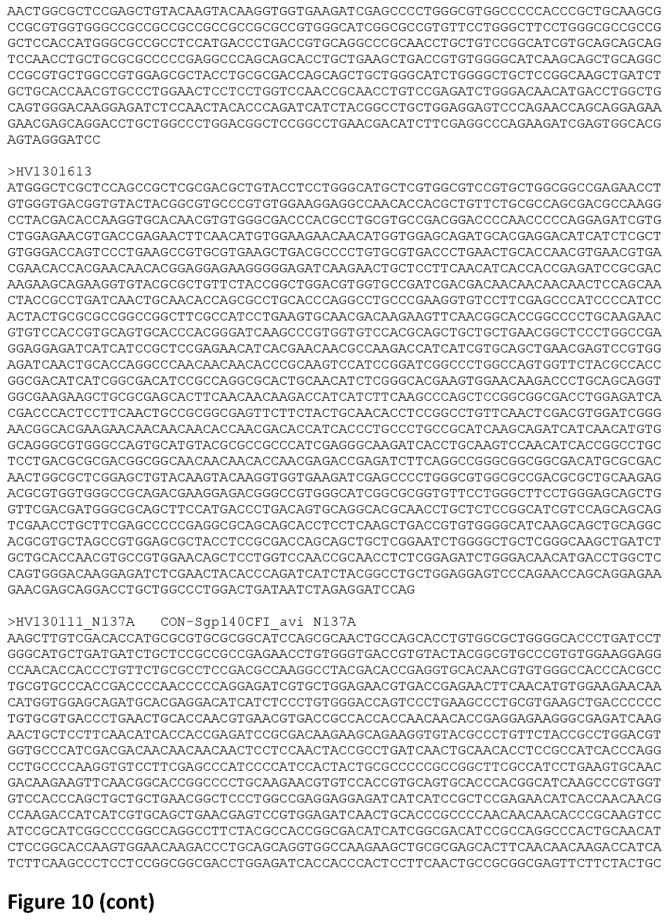

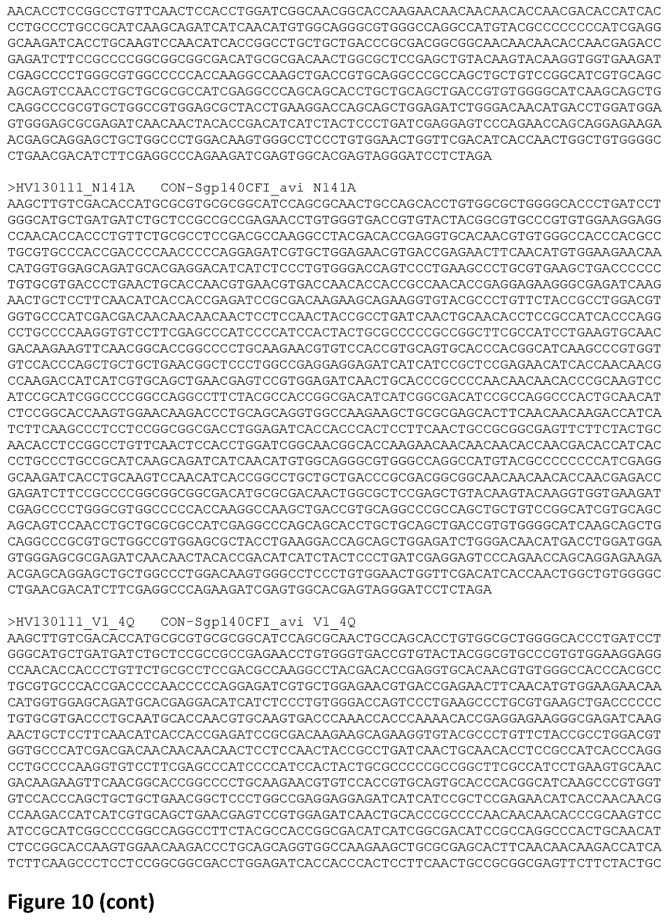

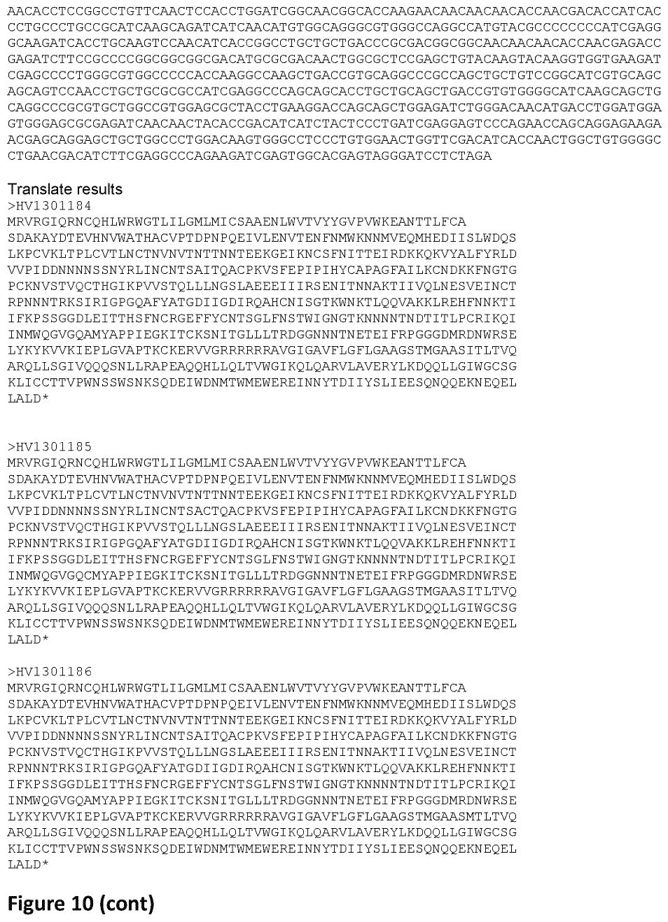

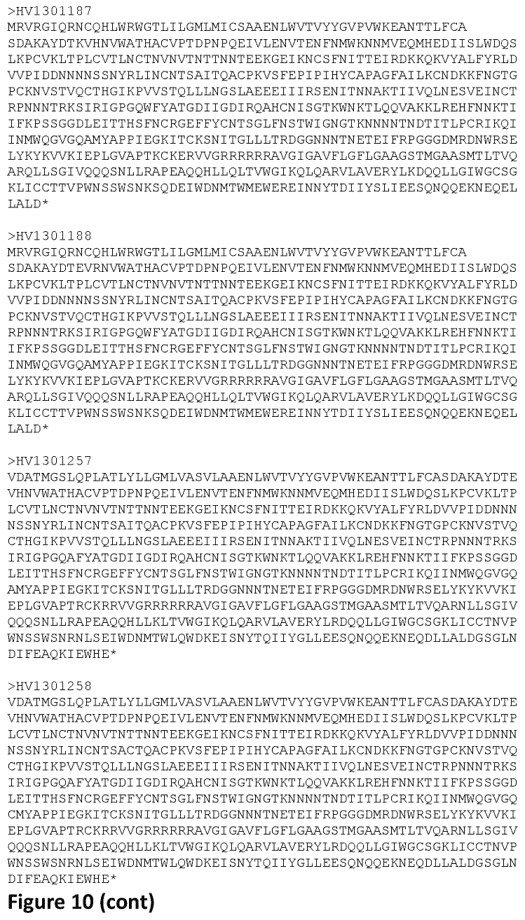

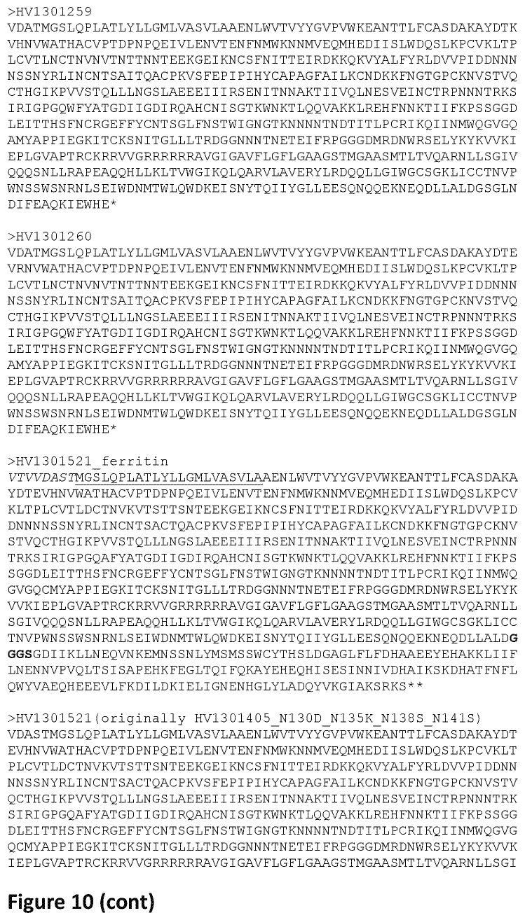

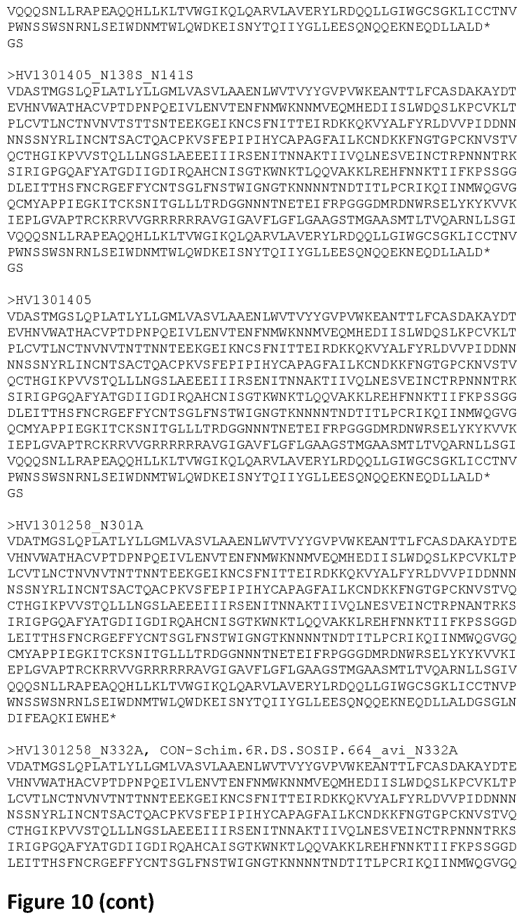

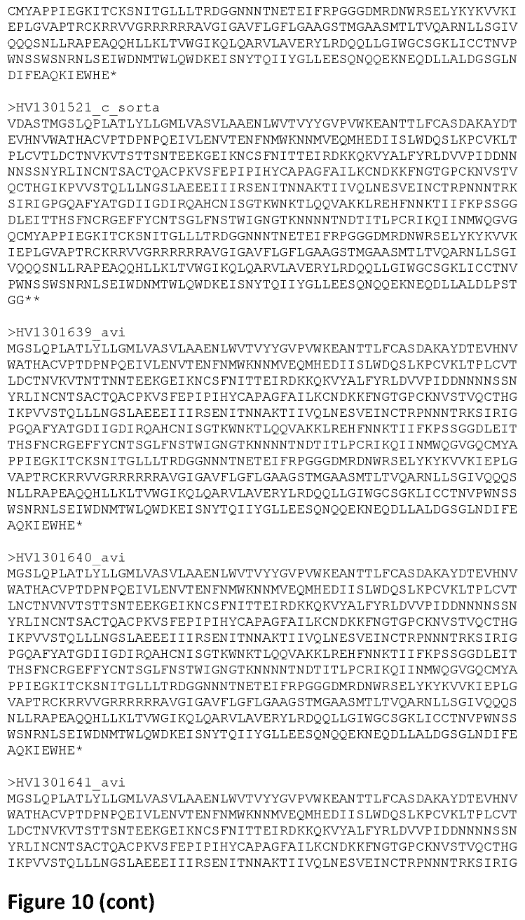

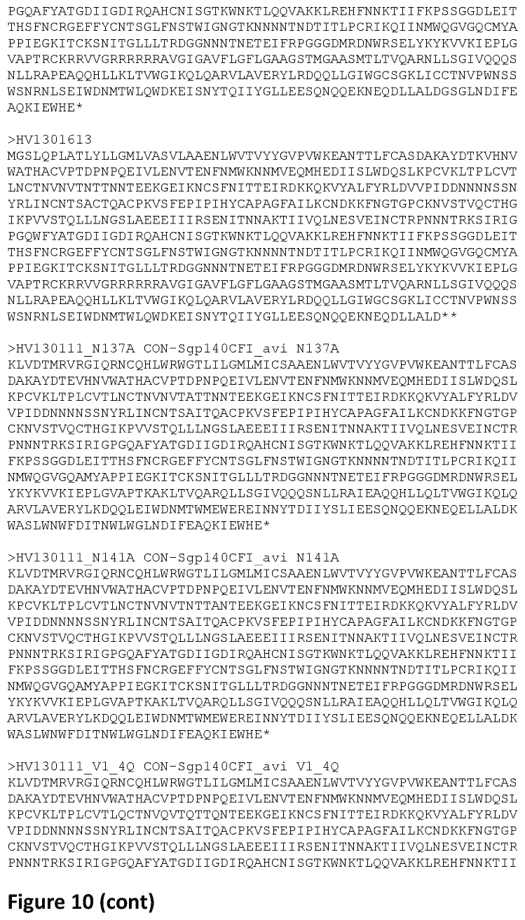

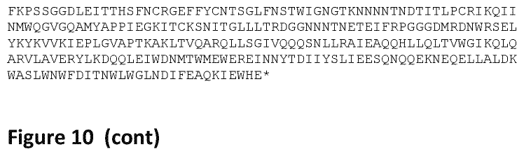

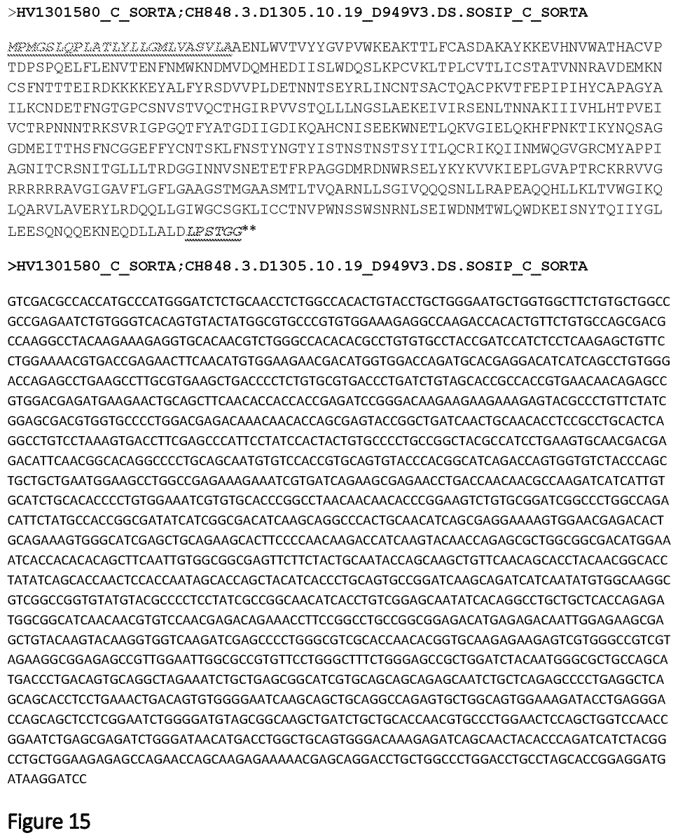

In certain aspects, the invention provides a recombinant HIV-1 envelope polypeptide as described here, wherein the polypeptide is a non-naturally occurring protomer designed to form an envelope trimer. The invention also provides nucleic acids encoding these recombinant polypeptides. Non-limiting examples of amino acids and nucleic acid of such protomers are referenced in Tables 1-3, and FIGS. 10-16.

In certain aspects the invention provides a recombinant trimer comprising three identical protomers of an envelope. In certain aspects, the invention provides an immunogenic composition comprising the recombinant trimer and a carrier, wherein the trimer comprises three identical protomers of an HIV-1 envelope as described herein. In certain aspects, the invention provides an immunogenic composition comprising nucleic acid encoding these recombinant HIV-1 envelope and a carrier.

Sequences/Clones

Described herein are nucleic and amino acids sequences of HIV-1 envelopes. The sequences for use as immunogens are in any suitable form. In certain embodiments, the described HIV-1 envelope sequences are gp160s. In certain embodiments, the described HIV-1 envelope sequences are gp120s. Other sequences, for example but not limited to stable SOSIP trimer designs, gp145s, gp140s, both cleaved and uncleaved, gp140 Envs with the deletion of the cleavage (C) site, fusion (F) and immunodominant (I) region in gp41—named as gp140ΔCFI (gp140CFI), gp140 Envs with the deletion of only the cleavage (C) site and fusion (F) domain—named as gp140ACF (gp140CF), gp140 Envs with the deletion of only the cleavage (C)—named gp140AC (gp140C) (See e.g. Liao et al. Virology 2006, 353, 268-282), gp150s, gp41s, which are readily derived from the nucleic acid and amino acid gp160 sequences. In certain embodiments the nucleic acid sequences are codon optimized for optimal expression in a host cell, for example a mammalian cell, a rBCG cell or any other suitable expression system.

An HIV-1 envelope has various structurally defined fragments/forms: gp160; gp140—including cleaved gp140 and uncleaved gp140 (gp140C), gp140CF, or gp140CFL gp120 and gp41. A skilled artisan appreciates that these fragments/forms are defined not necessarily by their crystal structure, but by their design and bounds within the full length of the gp160 envelope. While the specific consecutive amino acid sequences of envelopes from different strains are different, the bounds and design of these forms are well known and characterized in the art.

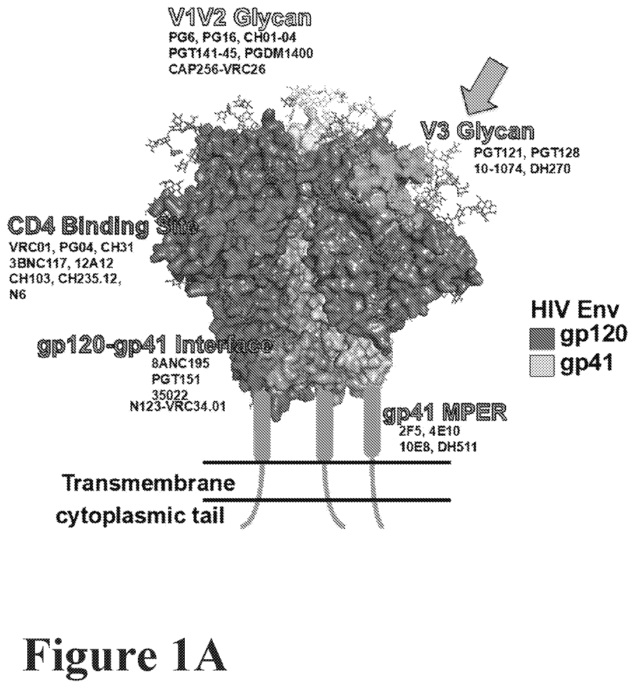

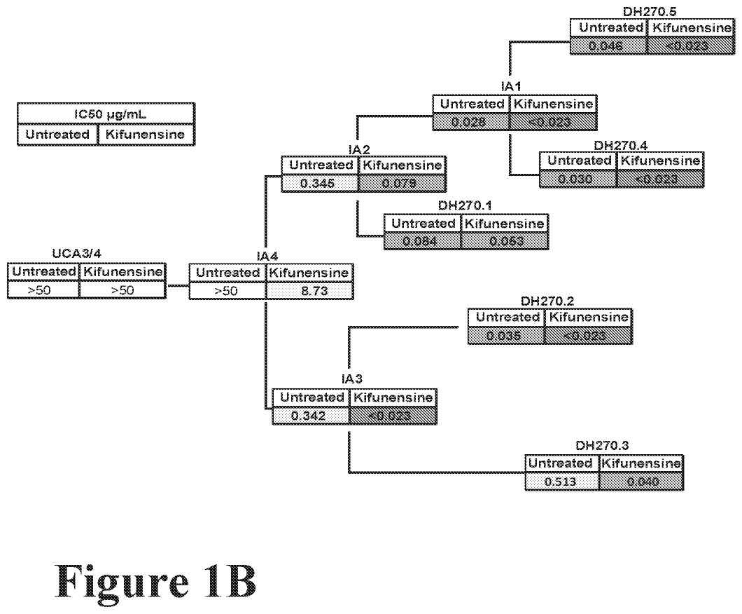

For example, it is well known in the art that during its transport to the cell surface, the gp160 polypeptide is processed and proteolytically cleaved to gp120 and gp41 proteins. Cleavages of gp160 to gp120 and gp41 occurs at a conserved cleavage site “REKR.” See Chakrabarti et al. Journal of Virology vol. 76, pp. 5357-5368 (2002) see for example FIG. 1, and Second paragraph in the Introduction on p. 5357; Binley et al. Journal of Virology vol. 76, pp. 2606-2616 (2002) for example at Abstract; Gao et al. Journal of Virology vol. 79, pp. 1154-1163 (2005); Liao et al. Virology vol. 353(2): 268-282 (2006).

The role of the furin cleavage site was well understood both in terms of improving cleave efficiency, see Binley et al. supra, and eliminating cleavage, see Bosch and Pawlita, Virology 64 (5):2337-2344 (1990); Guo et al. Virology 174: 217-224 (1990); McCune et al. Cell 53:55-67 (1988); Liao et al. J Virol. Apr;87(8):4185-201 (2013).

Likewise, the design of gp140 envelope forms is also well known in the art, along with the various specific changes which give rise to the gp140C (uncleaved envelope), gp140CF and gp140CFI forms. Envelope gp140 forms are designed by introducing a stop codon within the gp41 sequence. See Chakrabarti et al. at FIG. 1.

Envelope gp140C refers to a gp140 HIV-1 envelope design with a functional deletion of the cleavage (C) site, so that the gp140 envelope is not cleaved at the furin cleavage site. The specification describes cleaved and uncleaved forms, and various furin cleavage site modifications that prevent envelope cleavage are known in the art. In some embodiments of the gp140C form, two of the R residues in and near the furin cleavage site are changed to E, e.g., RRVVEREKR is changed to ERVVEREKE, and is one example of an uncleaved gp140 form. Another example is the gp140C form which has the REKR site changed to SEKS. See supra for references.

Envelope gp140CF refers to a gp140 HIV-1 envelope design with a deletion of the cleavage (C) site and fusion (F) region. Envelope gp140CFI refers to a gp140 HIV-1 envelope design with a deletion of the cleavage (C) site, fusion (F) and immunodominant (I) region in gp41. See Chakrabarti et al. Journal of Virology vol. 76, pp. 5357-5368 (2002) see for example FIG. 1, and Second paragraph in the Introduction on p. 5357; Binley et al. Journal of Virology vol. 76, pp. 2606-2616 (2002) for example at Abstract; Gao et al. Journal of Virology vol. 79, pp. 1154-1163 (2005); Liao et al. Virology vol. 353(2): 268-282 (2006).

In certain embodiments, the envelope design in accordance with the present invention involves deletion of residues (e.g., 5-11, 5, 6, 7, 8, 9, 10, or 11 amino acids) at the N-terminus. For delta N-terminal design, amino acid residues ranging from 4 residues or even fewer to 14 residues or even more are deleted. These residues are between the maturation (signal peptide, which can be readily determined by a skilled artisan) and “VPVXXXX . . . ”. In case of ConS Env as an example, amino acids (italicized and underlined in the below sequence) were deleted between the signal peptide and the

VPVXXX..:
MGSLQPLATLYLLGMLVASVLAAENLWVTVYYGVPVWKEANTT...

(rest of envelope sequence is indicated as “. . . ”). In certain embodiments, the invention relates generally to an immunogen, gp160, gp120 or gp140, without an N-terminal Herpes Simplex gD tag substituted for amino acids of the N-terminus of gp120, with an HIV leader sequence (or other leader sequence), and without the original about 4 to about 25, for example 4, 5, 6, 7, 8, 9, 10, 11, 12, 13, 14, 15, 16, 17, 18, 19, 20, 21, 22, 23, 24, or 25 amino acids of the N-terminus of the envelope (e.g. gp120). See WO2013/006688, e.g. at pages 10-12, the contents of which publication is hereby incorporated by reference in its entirety.

The general strategy of deletion of N-terminal amino acids of envelopes results in proteins, for example gp120s, expressed in mammalian cells that are primarily monomeric, as opposed to dimeric, and, therefore, solves the production and scalability problem of commercial gp120 Env vaccine production. In other embodiments, the amino acid deletions at the N-terminus result in increased immunogenicity of the envelopes.

In certain embodiments, the invention provides envelope sequences, amino acid sequences and the corresponding nucleic acids, and in which the V3 loop is substituted with the following V3 loop sequence TRPNNNTRKSIRIGPGQTFY ATGDIIGNIRQAH. This substitution of the V3 loop reduced product cleavage and improves protein yield during recombinant protein production in CHO cells.

In certain aspects, the invention provides composition and methods which use a selection of Envs, as gp120s, gp 140s cleaved and uncleaved, gp145s, gp150s and gp160s, stabilized and/or multimerized trimers, as proteins, DNAs, RNAs, or any combination thereof, administered as primes and boosts to elicit immune response. Envs as proteins would be co-administered with nucleic acid vectors containing Envs to amplify antibody induction. In certain embodiments, the compositions and methods include any immunogenic HIV-1 sequences to give the best coverage for T cell help and cytotoxic T cell induction. In certain embodiments, the compositions and methods include mosaic and/or consensus HIV-1 genes to give the best coverage for T cell help and cytotoxic T cell induction. In certain embodiments, the compositions and methods include mosaic group M and/or consensus genes to give the best coverage for T cell help and cytotoxic T cell induction. In some embodiments, the mosaic genes are any suitable gene from the HIV-1 genome. In some embodiments, the mosaic genes are Env genes, Gag genes, Pol genes, Nef genes, or any combination thereof. See e.g. U.S. Pat. No. 7,951,377. In some embodiments the mosaic genes are bivalent mosaics. In some embodiments the mosaic genes are trivalent. In some embodiments, the mosaic genes are administered in a suitable vector with each immunization with Env gene inserts in a suitable vector and/or as a protein. In some embodiments, the mosaic genes, for example as bivalent mosaic Gag group M consensus genes, are administered in a suitable vector, for example but not limited to HSV2, would be administered with each immunization with Env gene inserts in a suitable vector, for example but not limited to HSV-2.

In certain aspects the invention provides compositions and methods of Env genetic immunization either alone or with Env proteins to recreate the swarms of evolved viruses that have led to bnAb induction. Nucleotide-based vaccines offer a flexible vector format to immunize against virtually any protein antigen. Currently, two types of genetic vaccination are available for testing—DNAs and mRNAs.

In certain aspects the invention contemplates using immunogenic compositions wherein immunogens are delivered as DNA. See Graham BS, Enama ME, Nason MC, Gordon I J, Peel S A, et al. (2013) DNA Vaccine Delivered by a Needle-Free Injection Device Improves Potency of Priming for Antibody and CD8+ T-Cell Responses after rAd5 Boost in a Randomized Clinical Trial. PLoS ONE 8(4): e59340, page 9. Various technologies for delivery of nucleic acids, as DNA and/or RNA, so as to elicit immune response, both T-cell and humoral responses, are known in the art and are under developments. In certain embodiments, DNA can be delivered as naked DNA. In certain embodiments, DNA is formulated for delivery by a gene gun. In certain embodiments, DNA is administered by electroporation, or by a needle-free injection technologies, for example but not limited to Biojector® device. In certain embodiments, the DNA is inserted in vectors. The DNA is delivered using a suitable vector for expression in mammalian cells. In certain embodiments the nucleic acids encoding the envelopes are optimized for expression. In certain embodiments DNA is optimized, e.g. codon optimized, for expression. In certain embodiments, the nucleic acids are optimized for expression in vectors and/or in mammalian cells. In non-limiting embodiments these are bacterially derived vectors, adenovirus based vectors, rAdenovirus (e.g. Barouch D H, et al. Nature Med. 16: 319-23, 2010), recombinant mycobacteria (e.g. rBCG or M smegmatis) (Yu, J S et al. Clinical Vaccine Immunol. 14: 886-093,2007; ibid 13: 1204-11,2006), and recombinant vaccinia type of vectors (Santra S. Nature Med. 16: 324-8, 2010), for example but not limited to ALVAC, replicating (Kibler K V et al., PLoS One 6: e25674, 2011 Nov. 9.) and non-replicating (Perreau M et al. J. virology 85: 9854-62, 2011) NYVAC, modified vaccinia Ankara (MVA)), adeno-associated virus, Venezuelan equine encephalitis (VEE) replicons, Herpes Simplex Virus vectors, and other suitable vectors.

In certain aspects, the invention contemplates using immunogenic compositions wherein immunogens are delivered as DNA or RNA in suitable formulations. Various technologies which contemplate using DNA or RNA, or may use complexes of nucleic acid molecules and other entities to be used in immunization. In certain embodiments, DNA or RNA is administered as nanoparticles consisting of low dose antigen-encoding DNA formulated with a block copolymer (amphiphilic block copolymer 704). See Cany et al., Journal of Hepatology 2011 vol. 54 j 115-121; Arnaoty et al., Chapter 17 in Yves Bigot (ed.), Mobile Genetic Elements: Protocols and Genomic Applications, Methods in Molecular Biology, vol. 859, pp293-305 (2012); Arnaoty et al. (2013) Mol Genet Genomics. 2013 Aug;288(7-8):347-63. Nanocarrier technologies called Nanotaxi® for immunogenic macromolecules (DNA, RNA, Protein) delivery are under development. See for example technologies developed by incellart.

mRNA

In some embodiments the antigens are nucleic acids, including but not limited to mRNAs which could be modified and/or unmodified. See U.S. Pub 20180028645A1, U.S. Pub 20170369532, U.S. Pub 20090286852, U.S. Pub 20130111615, U.S. Pub 20130197068, U.S. Pub 20130261172, U.S. Pub 20150038558, U.S. Pub 20160032316, U.S. Pub 20170043037, U.S. Pub 20170327842, each content is incorporated by reference in its entirety. mRNAs delivered in LNP formulations have advantages over non-LNPs formulations. See U.S. Pub 20180028645A1.

In certain aspects the invention contemplates using immunogenic compositions wherein immunogens are delivered as recombinant proteins. Various methods for production and purification of recombinant proteins, including trimers such as but not limited to SOSIP based trimers, suitable for use in immunization are known in the art. In certain embodiments recombinant proteins are produced in CHO cells.

It is readily understood that the envelope glycoproteins referenced in various examples and figures comprise a signal/leader sequence. It is well known in the art that HIV-1 envelope glycoprotein is a secretory protein with a signal or leader peptide sequence that is removed during processing and recombinant expression (without removal of the signal peptide, the protein is not secreted). See for example Li et al. Control of expression, glycosylation, and secretion of HIV-1 gp120 by homologous and heterologous signal sequences. Virology 204(1):266-78 (1994) (“Li et al. 1994”), at first paragraph, and Li et al. Effects of inefficient cleavage of the signal sequence of HIV-1 gp120 on its association with calnexin, folding, and intracellular transport. PNAS 93:9606-9611 (1996) (“Li et al. 1996”), at 9609. Any suitable signal sequence could be used. In some embodiments the leader sequence is the endogenous leader sequence. Most of the gp120 and gp160 amino acid sequences include the endogenous leader sequence. In other non-limiting examples the leader sequence is human Tissue Plasminogen Activator (TPA) sequence, human CD5 leader sequence (e.g. MPMGSLQPLATLYLLGMLVASVLA). Most of the chimeric designs include CD5 leader sequence. A skilled artisan appreciates that when used as immunogens, and for example when recombinantly produced, the amino acid sequences of these proteins do not comprise the leader peptide sequences.

The immunogenic envelopes can also be administered as a protein prime and/or boost alone or in combination with a variety of nucleic acid envelope primes (e.g., HIV -1 Envs delivered as DNA expressed in viral or bacterial vectors).

Dosing of proteins and nucleic acids can be readily determined by a skilled artisan. A single dose of nucleic acid can range from a few nanograms (ng) to a few micrograms (μg) or milligram of a single immunogenic nucleic acid. Recombinant protein dose can range from a few μg micrograms to a few hundred micrograms, or milligrams of a single immunogenic polypeptide.

Administration: The compositions can be formulated with appropriate carriers using known techniques to yield compositions suitable for various routes of administration. In certain embodiments the compositions are delivered via intramascular (IM), via subcutaneous, via intravenous, via nasal, via mucosal routes, or any other suitable route of immunization.

The compositions can be formulated with appropriate carriers and adjuvants using techniques to yield compositions suitable for immunization. The compositions can include an adjuvant, such as, for example but not limited to, alum, poly IC, MF-59 or other squalene-based adjuvant, ASOIB, or other liposomal based adjuvant suitable for protein or nucleic acid immunization. In certain embodiments, the adjuvant is GSK AS01E adjuvant containing MPL and QS21. This adjuvant has been shown by GSK to be as potent as the similar adjuvant ASO1B but to be less reactogenic using HBsAg as vaccine antigen [Leroux-Roels et al., IABS Conference, April 2013]. In certain embodiments, TLR agonists are used as adjuvants. In certain embodiments, the adjuvant is 3M052. In other embodiment, adjuvants which break immune tolerance are included in the immunogenic compositions.

In certain embodiments, the compositions and methods comprise any suitable agent or immune modulation, which could modulate mechanisms of host immune tolerance and release of the induced antibodies. In non-limiting embodiments modulation includes PD-1 blockade; T regulatory cell depletion; CD4OL hyperstimulation; soluble antigen administration, wherein the soluble antigen is designed such that the soluble agent eliminates B cells targeting dominant epitopes, or a combination thereof. In certain embodiments, an immunomodulatory agent is administered in at time and in an amount sufficient for transient modulation of the subject's immune response so as to induce an immune response which comprises broad neutralizing antibodies against HIV-1 envelope. Non-limiting examples of such agents is any one of the agents described herein: e.g. chloroquine (CQ), PTP1B Inhibitor—CAS 765317-72-4—Calbiochem or MSI 1436 clodronate or any other bisphosphonate; a Foxol inhibitor, e.g. 344355|Foxol Inhibitor, AS1842856—Calbiochem; Gleevac, anti-CD25 antibody, anti-CCR4 Ab, an agent which binds to a B cell receptor for a dominant HIV-1 envelope epitope, or any combination thereof. In non-limiting embodiments, the modulation includes administering an anti-CTLA4 antibody, OX-40 agonists, or a combination thereof. Non-limiting examples are of CTLA-1 antibody are ipilimumab and tremelimumab. In certain embodiments, the methods comprise administering a second immunomodulatory agent, wherein the second and first immunomodulatory agents are different.

Multimeric Envelopes

Presentation of antigens as particulates reduces the B cell receptor affinity necessary for signal transduction and expansion (See Baptista et al. EMBO J. 2000 Feb. 15; 19(4): 513-520). Displaying multiple copies of the antigen on a particle provides an avidity effect that can overcome the low affinity between the antigen and B cell receptor. The initial B cell receptor specific for pathogens can be low affinity, which precludes vaccines from being able to stimulate and expand B cells of interest. In particular, very few naïve B cells from which HIV-1 broadly neutralizing antibodies arise can bind to soluble HIV-1 Envelope. Provided are envelopes, including but not limited to trimers as particulate, high-density array on liposomes or other particles, for example but not limited to nanoparticles. See e.g. He et al. Nature Communications 7, Article number: 12041 (2016), doi:10.1038/ncomms12041; Bamrungsap et al. Nanomedicine, 2012, 7 (8), 1253-1271.

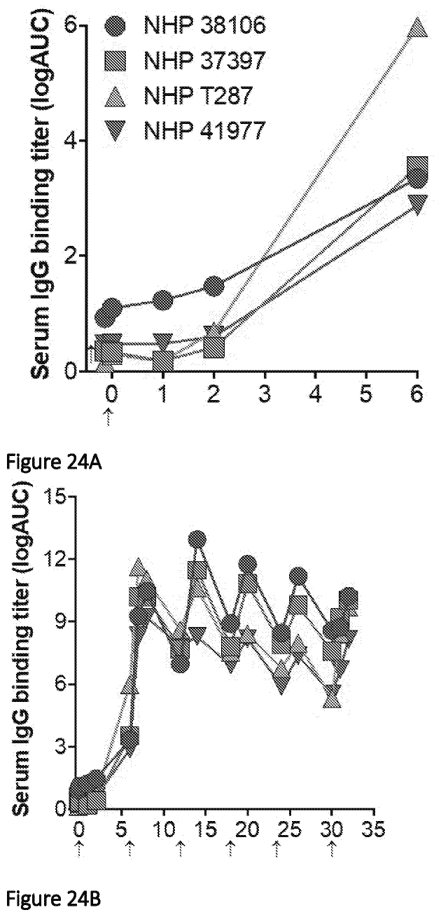

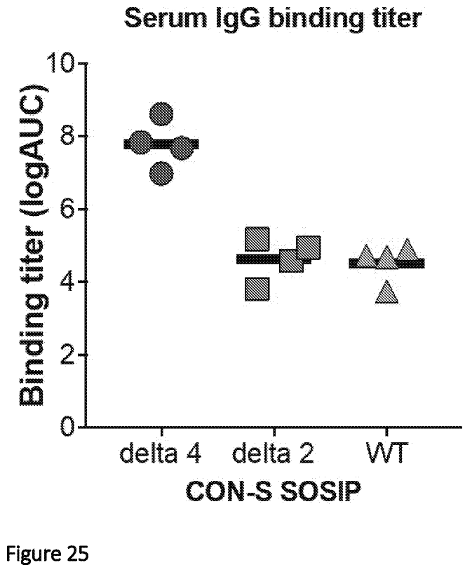

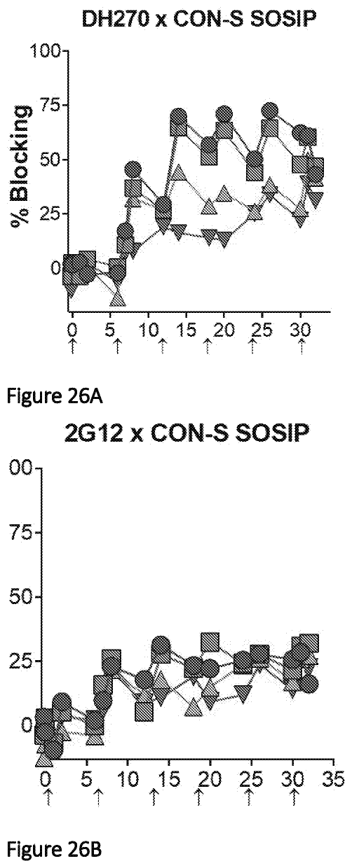

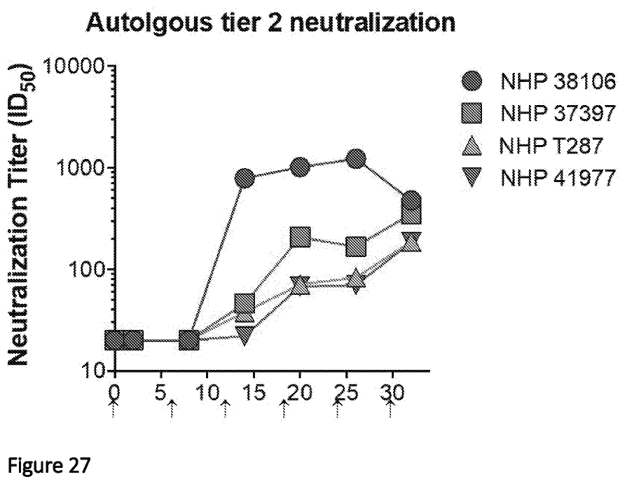

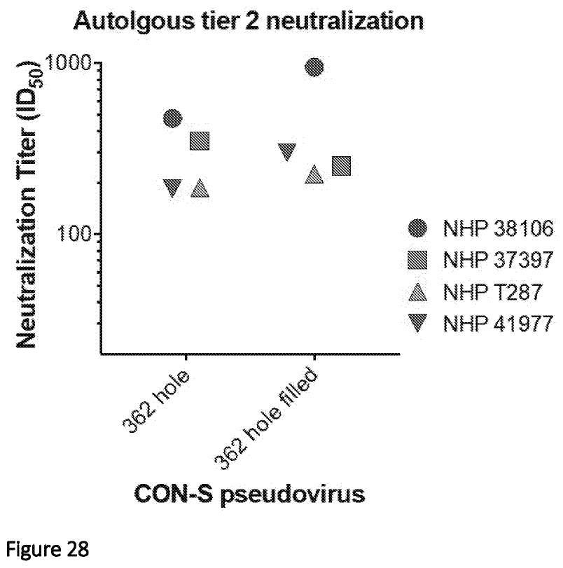

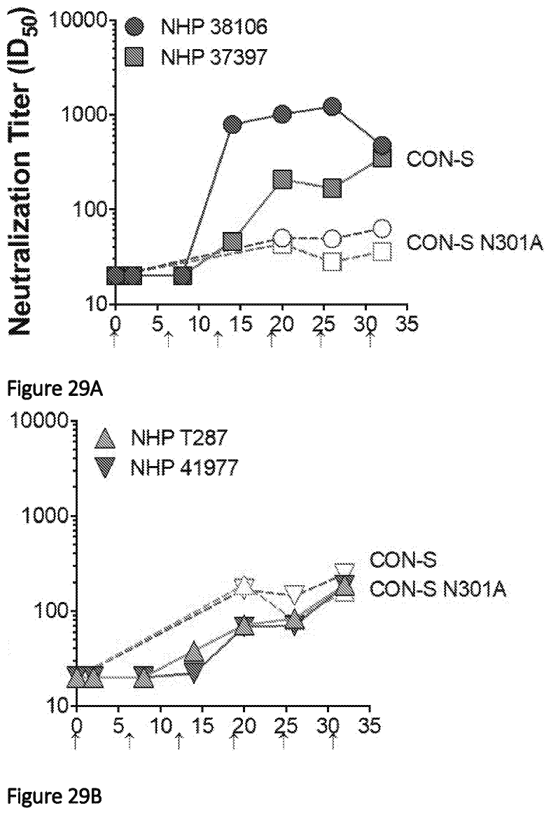

To improve the interaction between the naïve B cell receptor and immunogens, envelope designed can be created to wherein the envelope is presented on particles, e.g. but not limited to nanoparticle. In some embodiments, the HIV-1 Envelope trimer could be fused to ferritin. Ferritin protein self assembles into a small nanoparticle with three-fold axis of symmetry. At these axes the envelope protein is fused. Therefore, the assembly of the three-fold axis also clusters three HIV-1 envelope protomers together to form an envelope trimer. Each ferritin particle has 8 axes which equates to 8 trimers being displayed per particle. See e.g. Sliepen et al. Retrovirology201512:82, DOI: 10.1186/s12977-015-0210-4; See also FIG. 24H-J.

Another approach to multimerize expression constructs uses staphylococcus Sortase A transpeptidase ligation to conjugate inventive envelope trimers to cholesterol. The trimers can then be embedded into liposomes via the conjugated cholesterol. To conjugate the trimer to cholesterol either a C-terminal LPXTG(X1) tag, wherein X1 could be a Glycine (G), or a N-terminal pentaglycine repeat tag is added to the envelope trimer gene. Cholesterol is also synthesized with these two tags. Sortase A is then used to covalently bond the tagged envelope to the cholesterol. The sortase A-tagged trimer protein can also be used to conjugate the trimer to other peptides, proteins, or fluorescent labels. In non-limiting embodiments, the sortase A tagged trimers are conjugated to ferritin to form nanoparticles.

Multimerization of SOSIP trimers. Previous strategies for multimerizing SOSIP Env trimers have been successful, but limited in their multivalency. The B cell receptor recognizes and internalizes low-affinity antigens at a greater magnitude when the low-affinity antigen is presented as a multimeric particle as opposed to monomeric protein in solution (Batista FD, Neuberger MS. B cells extract and present immobilized antigen: implications for affinity discrimination. EMBO J. 2000;19(4):513-20). In vivo, the multimerization of HIV-1 Env has improved neutralizing antibody titers in rabbits (Ingale J, Stano A, Guenaga J, Sharma SK, Nemazee D, Zwick MB, et al. High-Density Array of Well-Ordered HIV-1 Spikes on Synthetic Liposomal Nanoparticles Efficiently Activate B Cells. Cell Rep. 2016;15(9):1986-99.) and monkeys (Martinez-Murillo P, Tran K, Guenaga J, Lindgren G, Adori M, Feng Y, et al. Particulate Array of Well-Ordered HIV Clade C Env Trimers Elicits Neutralizing Antibodies that Display a Unique V2 Cap Approach. Immunity. 2017;46(5):804-17 e7).

In certain embodiments, there are methods for expressing and purifying the Env trimers as multimers as ferritin nanoparticles. Purification of SOSIP gp140-ferritin fusion proteins can be complicated by the presence of well-folded and poorly folded trimeric Env on the same nanoparticle, so we developed a two-step ferritin assembly process where we first purified well-folded SOSIP gp140 trimers and separately purified ferritin nanoparticles. We then covalently link the SOSIP to ferritin via short sortase-A linker peptides (e.g. FIG. 17A). The presence of HIV-1 Env trimers on conjugated ferritin particles is confirmed with negative-stain electron microscopy.

The invention provides design of envelopes and trimer designs wherein the envelope comprises a linker which permits addition of another molecule, e.g. but not limited to a protein, such as but not limited to ferritin, or lipid, such as but not limited to cholesterol, via a Sortase A reaction. See e.g. Tsukiji, S. and Nagamune, T. (2009), Sortase-Mediated Ligation: A Gift from Gram-Positive Bacteria to Protein Engineering. ChemBioChem, 10: 787-798. doi:10.1002/cbic.200800724; Proft, T. Sortase-mediated protein ligation: an emerging biotechnology tool for protein modification and immobilisation. Biotechnol Lett (2010) 32: 1. doi:10.1007/s10529-009-0116-0; Lena Schmohl, Dirk Schwarzer, Sortase-mediated ligations for the site-specific modification of proteins, Current Opinion in Chemical Biology, Volume 22, October 2014, Pages 122-128, ISSN 1367-5931, dx.doi.org/10.1016/j.cbpa.2014.09.020; Tabata et al. Anticancer Res. 2015 August;35(8):4411-7; Pritz et al. J. Org. Chem. 2007, 72, 3909-3912.

The lipid modified envelopes and trimers could be formulated as liposomes. Any suitable liposome composition is contemplated.

Non-limiting embodiments of envelope designs for use in Sortase A reaction are shown in FIG. 24 B-D in WO2017151801 and FIGS. 47 B-C in WO2017/152146, incorporated by reference in its entirety.

Additional sortase linkers could be used so long as their position allows multimerization of the envelopes.

TABLE 1
summary of sequences

| | Amino acid, | | FIG./ |
|---|---|---|---|
| Name | nucleic acid | Design | Note |
| HV1301580_D230N_H289N_P291S; | Nt | | 12 |
| CH848.3.D1305.10.19_D949V3.DS.SOSIP_D230N_H289N_P291S | aa | | 13 |
| (glycan hole filled) | | | |
| >HV1301502_D1305V1; | Nt | | 12 |
| JRFL_SOSIPv6_V1_PNGS_D1305V1 | aa | | 13 |
| (V1 loop from 10.19) | | | |
| >HV1301405_D1305V1; | Nt | | 12 |
| CON-Schim.6R.DS.SOSIP.664_OPT_D1305V1 | aa | | 13 |
| (V1 loop from 10.19 isolate) | | | |
| >HV1301580_D230N_H289N_P291S; | Nt | | 12 |
| CH848.3.D1305.10.19_D949V3.DS.SOSIP_D230N_H289N_P291S | aa | | 13 |
| (glycan holes filled) | | | |
| >HV1301580; | Nt | 19CV3 | 12 |
| CH848.3.D1305.10.19_D949V3.DS.SOSIP (19CV3) | aa | | 13 |
| >HV1301509; | Nt | | 12 |
| CH0848.3.d1305.10.19gp160 | aa | | 13 |
| >HV1301503; | Nt | | 12 |
| CH848.3.D1305.10.19ch.DS.SOSIP.664 | aa | | 13 |
| >HV1301504; | Nt | | 12 |
| CH848.3.D1305.10.19ch.SOSIPv6 | aa | | 13 |
| >HV1301580_C_SORTA; | Aa | | 14 |
| CH848.3.D1305.10.19_D949V3.DS.SOSIP_C_SORTA | nt | | 14 |

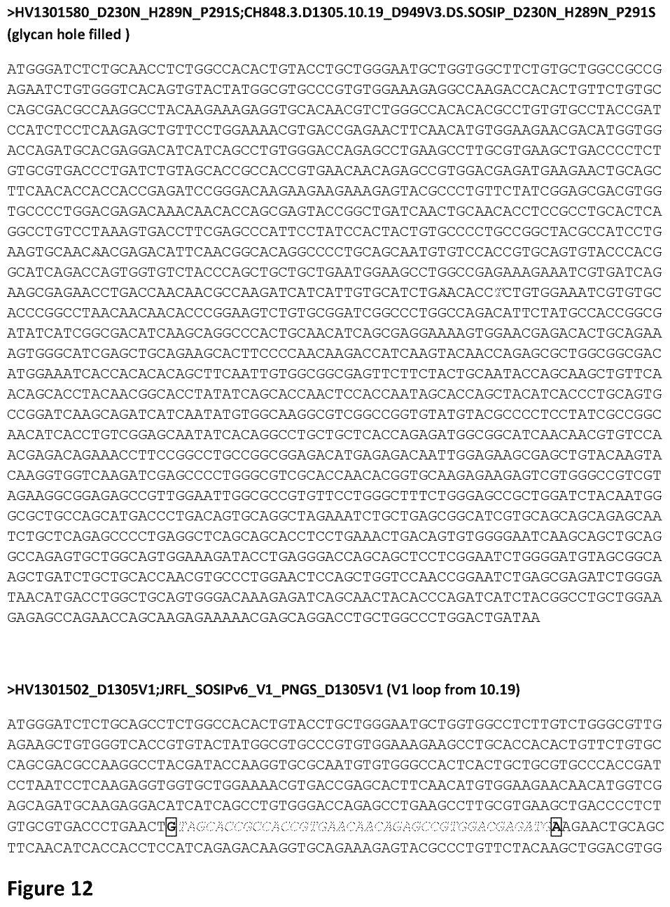

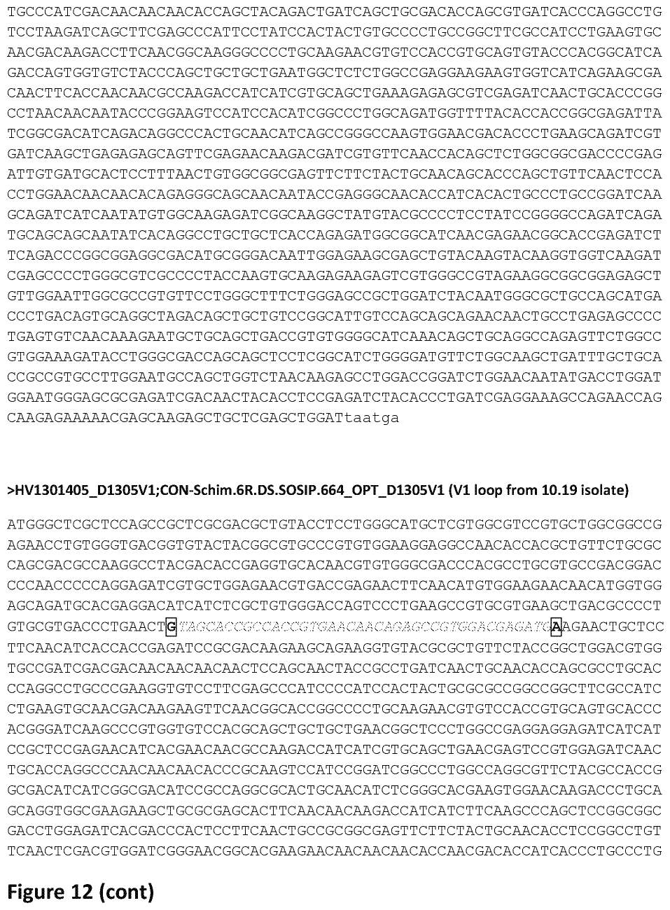

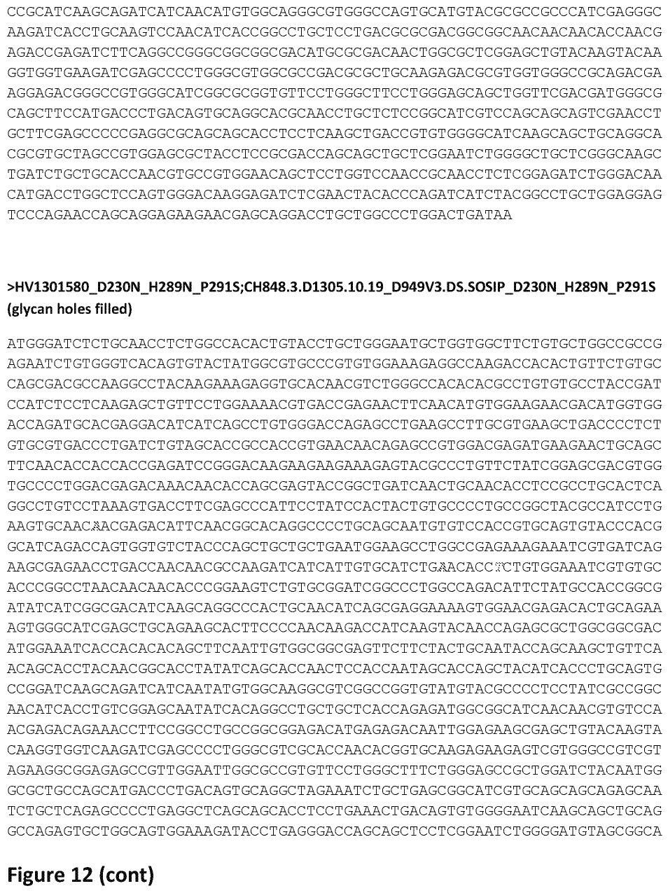

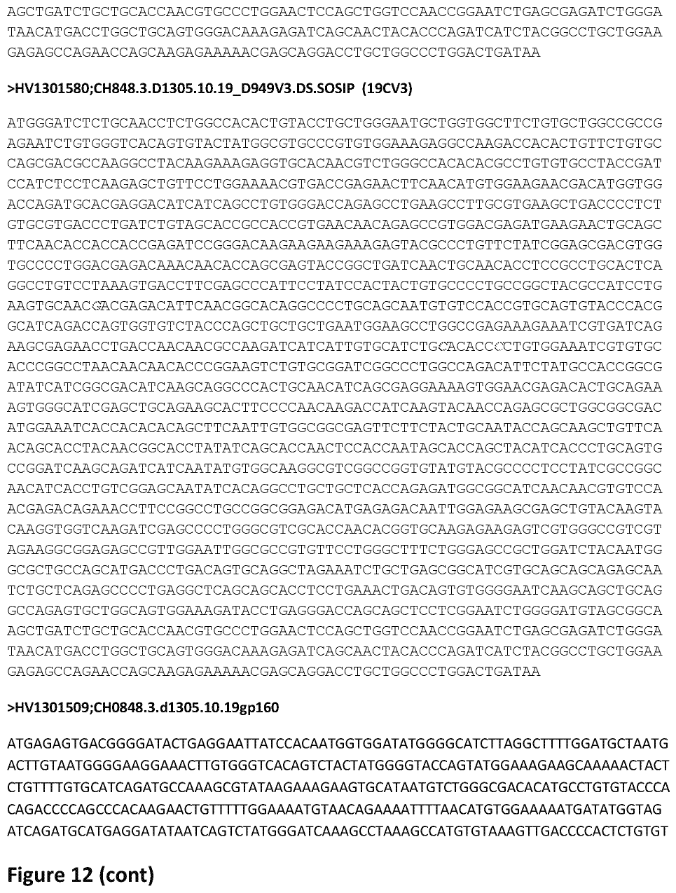

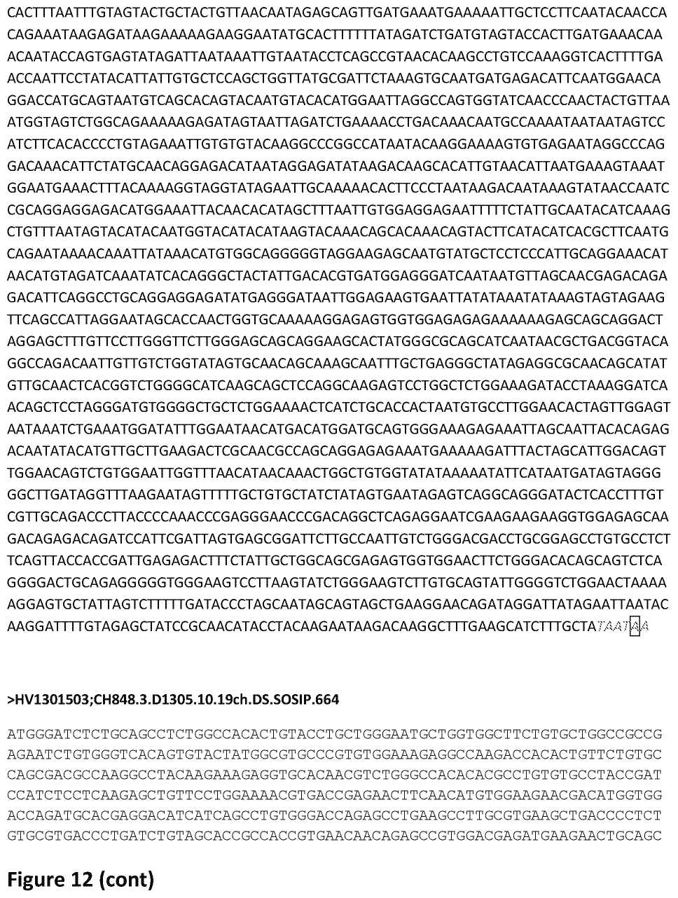

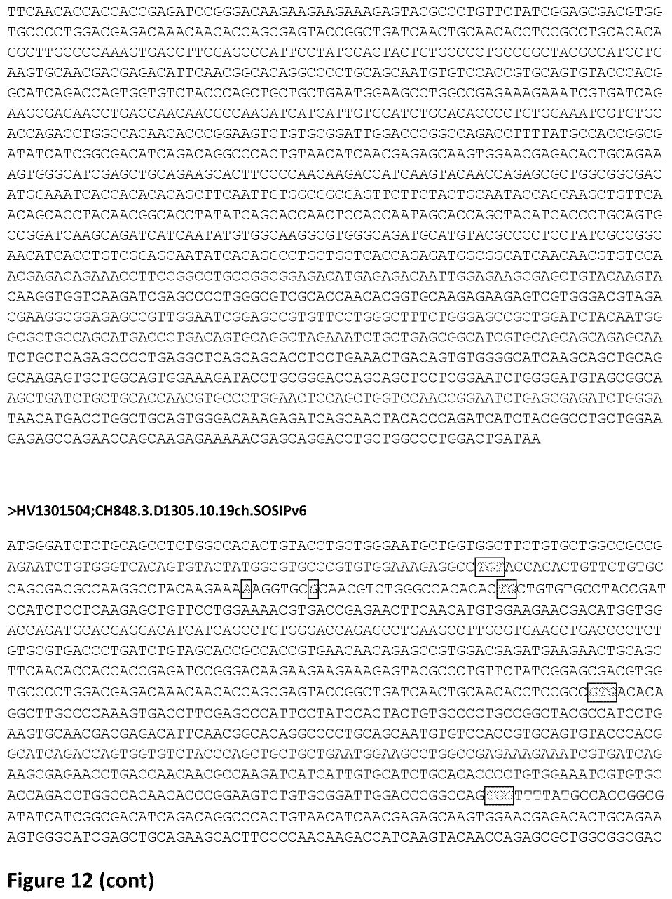

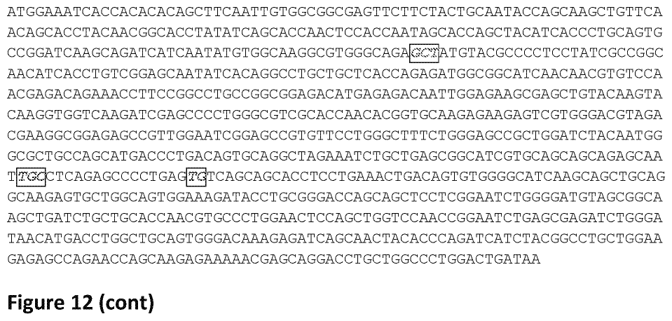

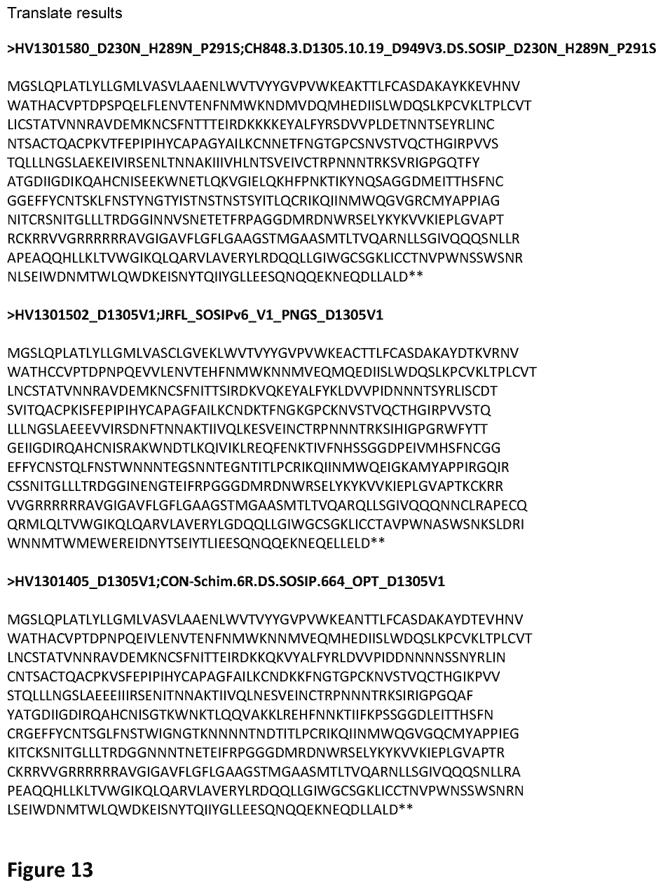

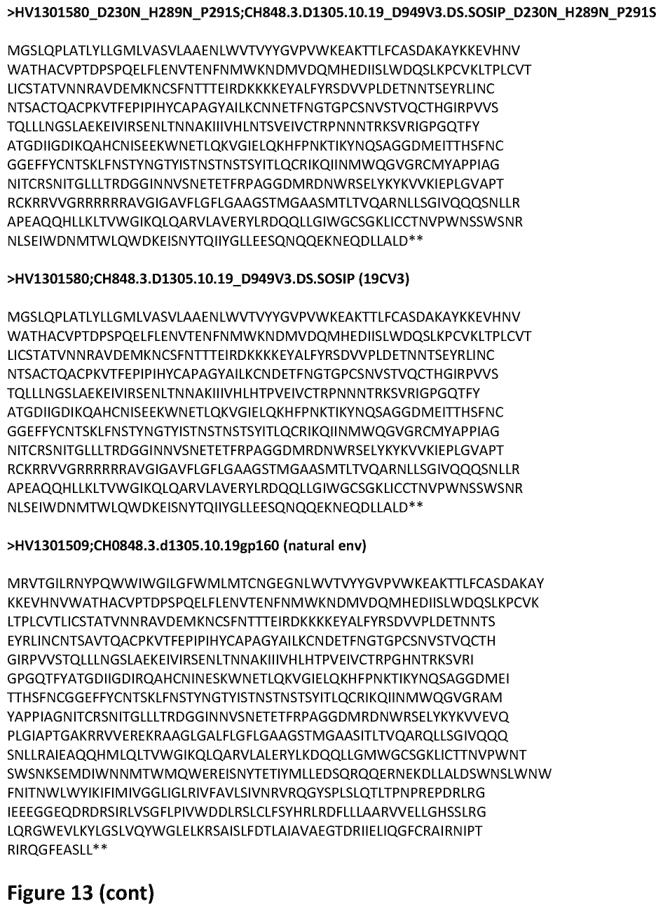

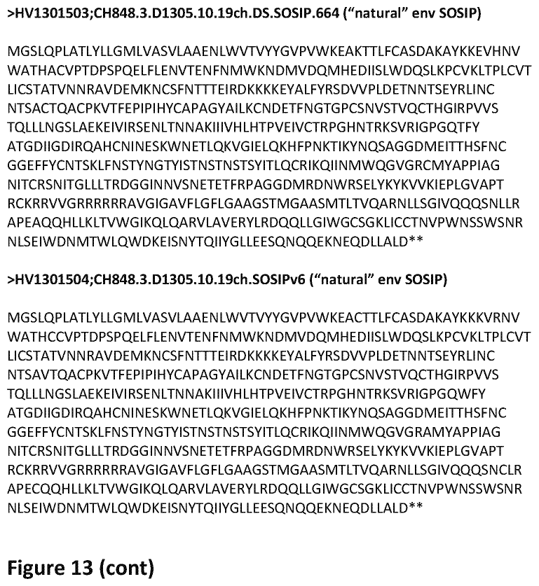

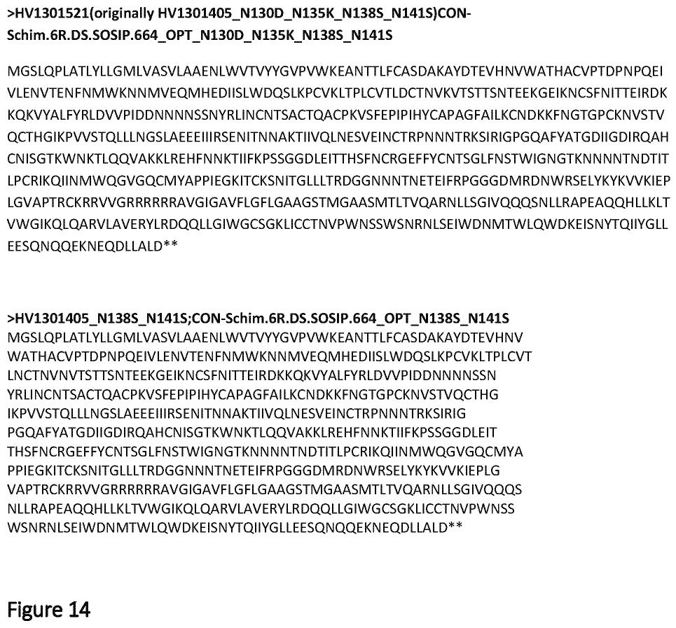

TABLE 2
Summary of mutations

| Envelope | Figure/SEQ ID No | V1 region | V3 glycosylation sites | UCA Ab binding |
|---|---|---|---|---|
| 10.17 | WO2017152146 | 17aa | N301 and N332 | |
| | and | | | |
| | W02018/161049 | | | |
| 10.17DT | WO2017152146 | 17aa N133D | N301 andN332 | DH270UCA |
| | and | N138T | | |
| | W02018/161049 | effectively | | |
| | | lacks | | |
| | | glycosylation | | |
| | | sites | | |
| 10.19 | FIG. 12 | 17aa V1 region | No glycosylation sites | CH01 UCA |
| | | lacks N133 and | at N295, N301, N332 | |
| | | N138 | | |
| | | glycosylation | | |
| | | sites | | |
| 10.19 plus | FIG. 12, 13, | 17aa V1 region | Add V3 regions from | CH01 UCA |
| V3 loop of | FIG. 14 | lacks N133 and | 10.17 has five aa | DH270UCA |
| 10.17 | | N138 | difference from 10.19 | VRC26 UCA |
| (19CV3) | | glycosylation | | |
| | | sites | | |
| 10.19 env | | | At least changes #2, | |
| based with | | | 4, 5, and/or “GDIR” | |
| fewer than | | | sequence | |
| five aa | | | | |
| changes | | | | |
| compared | | | | |
| to 19CV3 | | | | |
| ConS | FIG. 12, FIG. 13 | 17aa V1 region | | |
| | | (from envelope | | |
| | | 10.19) lacks | | |
| | | N133 andN138 | | |

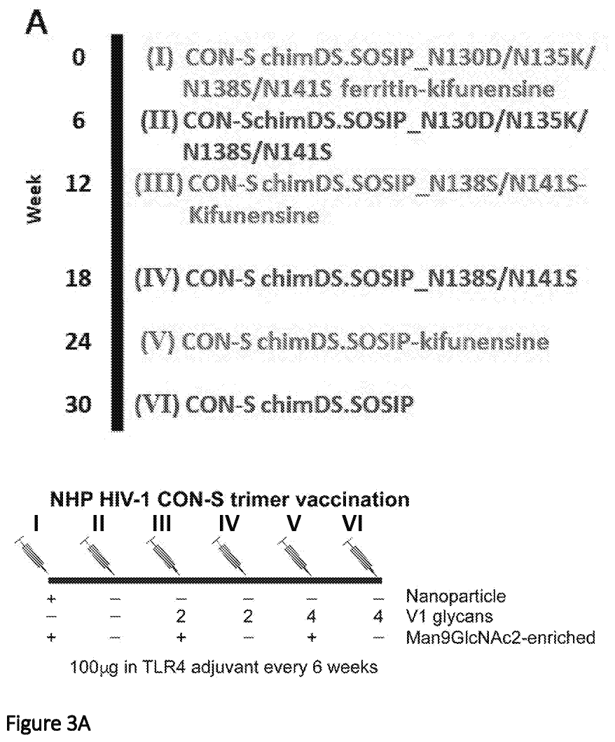

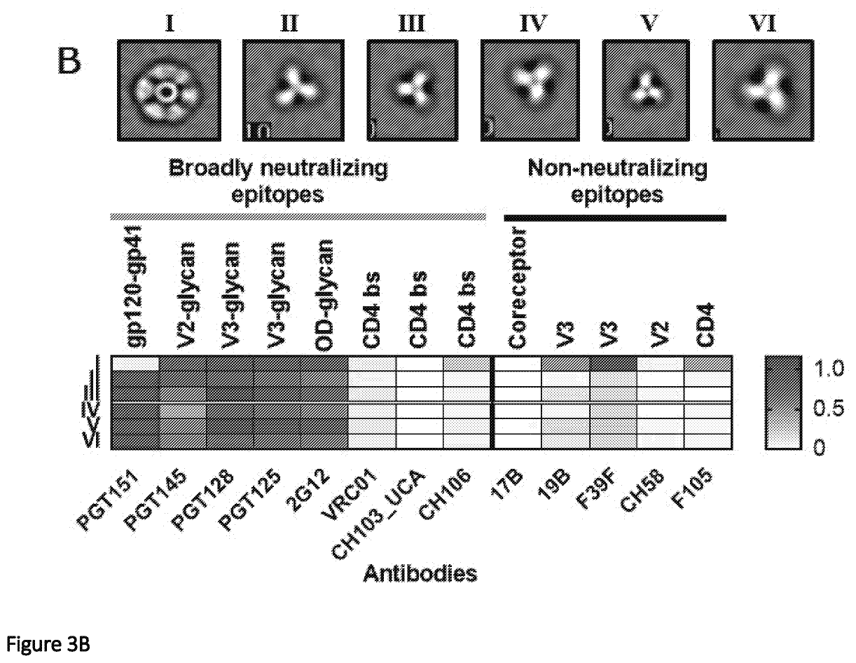

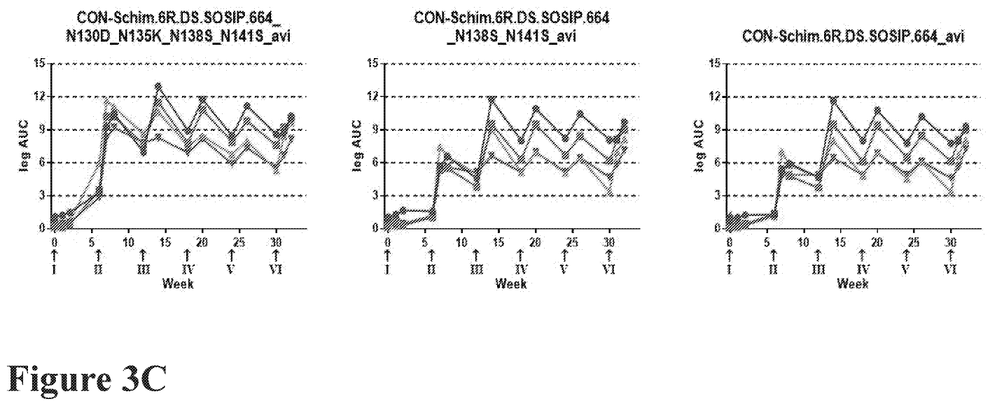

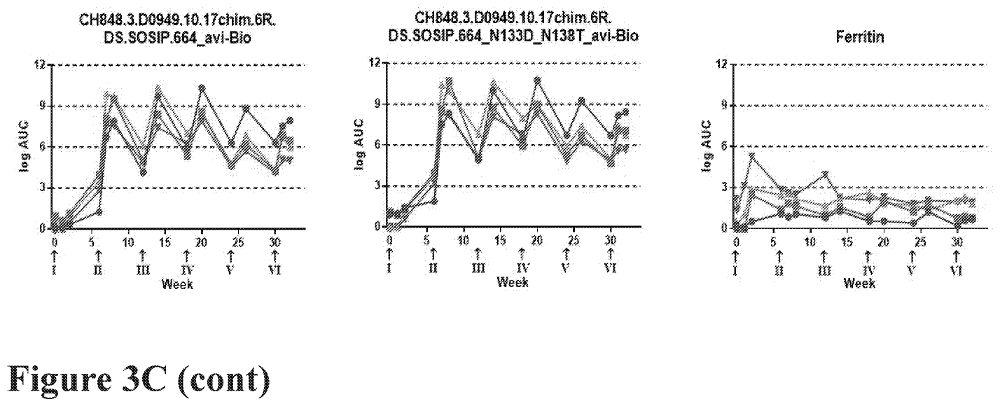

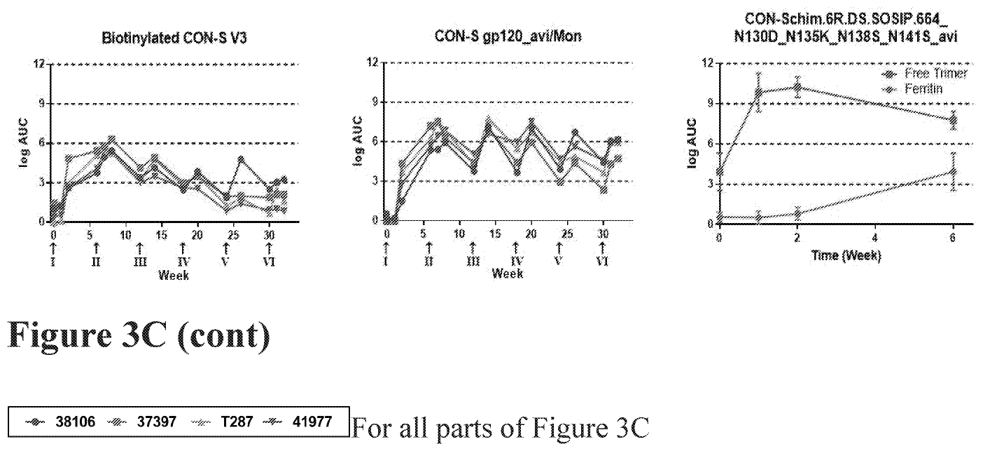

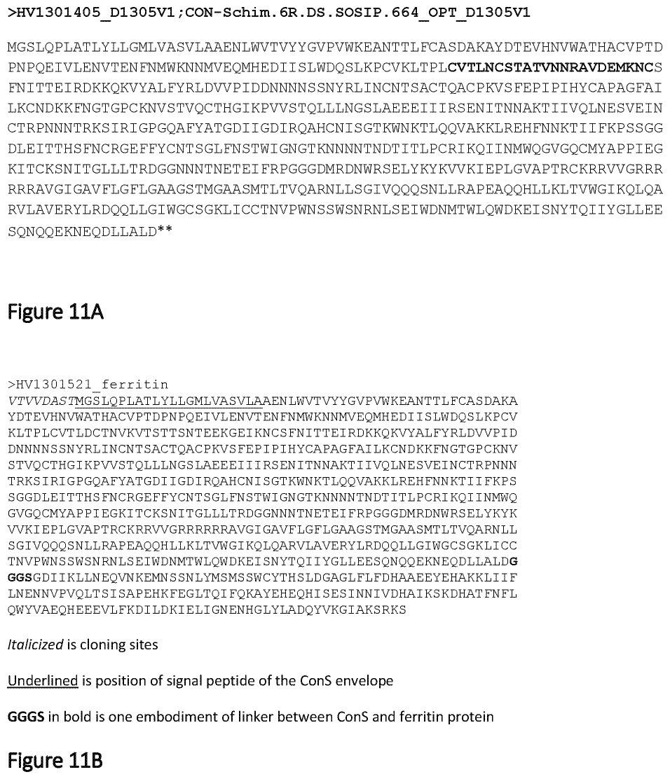

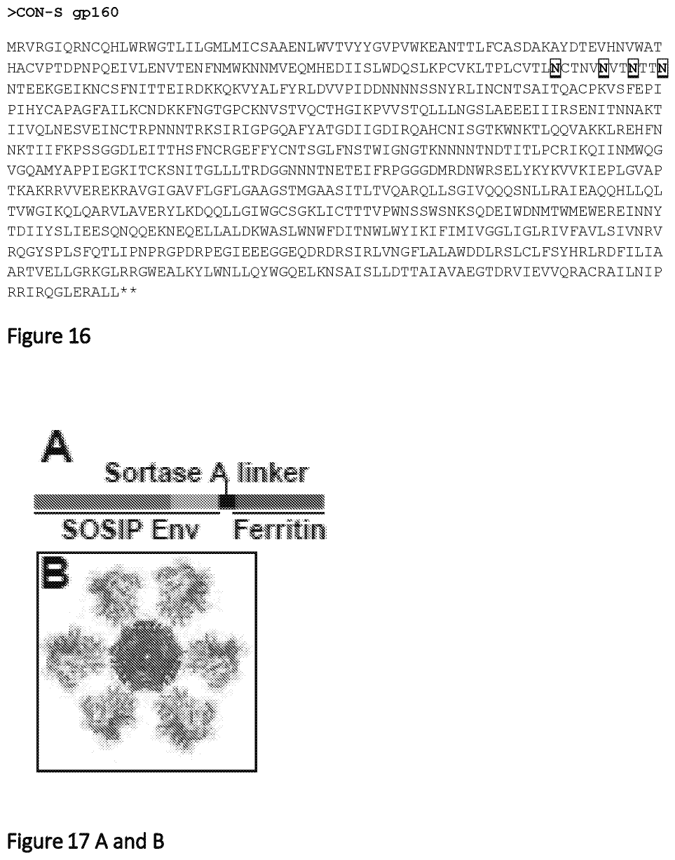

TABLE 3
Listing non-limiting embodiments of immunogens, correlating plasmid number (see FIGS. 10-16) and names

| Plasmid number | Protein name | Note | FIG. |
|---|---|---|---|
| HV1301184 | CON-S.6R.SOSIP.664 | | FIG. 10 |
| HV1301185 | CON-S.6R.DS.SOSIP.664 | | FIG. 10 |
| HV1301186 | CON-S.6R.SOSIP.664.v3.1 | | FIG. 10 |
| HV1301187 | CON-S.6R.SOSIP.664.v4.1 | | FIG. 10 |
| HV1301188 | CON-S.6R.SOSIP.664.v4.2 | | FIG. 10 |
| HV1301257 | CON-Schim.6R.SOSIP.664_avi | | FIG. 10 |
| HV1301258 | CON-Schim.6R.DS.SOSIP.664_avi | | FIG. 10 |
| HV1301259 | CON-Schim.6R.SOSIP.664v4.1_avi | | FIG. 10 |
| HV1301260 | CON-Schim.6R.SOSIP.664v4.2_avi | | FIG. 10 |
| HV1301639_avi | CON-Schim.6R.DS.SOSIP.664_N130D_N135K_avi | | FIG. 10 |
| HV1301640_avi | CON-Schim.6R.DS.SOSIP.664_N138S_N141S_avi | | FIG. 10 |
| HV1301641_avi | CON-Schim.6R.DS.SOSIP.664_N130D_N135K_N138S_N141S_avi | | FIG. 10 |
| HV1301613 | CON-Schim.6R.DS.SOSIP.664v4.1_OPT | | FIG. 10 |
| HV1301521_ferritin | CON-Schim.6R.DS.SOSIP.664_OPT_N130D_N135K_N138S_N141S_ferritin | I in | FIG. 10 |
| | | FIG. 3A | FIG. 11B |
| | CON-Schim.6R.DS.SOSIP.664_OPT_N130X_N135X_N138X_N141X_ferritin | | |
| HV1301521 | CON-Schim.6R.DS.SOSIP.664_OPT_N130D_N135K_N138S_N141S | II in | FIG. 10 |
| | | FIG. 3A | |
| | CON-Schim.6R.DS.SOSIP.664_OPT_N130X_N135X_N138X_N141X | | |
| HV1301405_N138S_N141S | CON-Schim.6R.DS.SOSIP.664_OPT_N138S_N141S | III and | FIG. 10 |
| | | IV in | |
| | | FIG. 3A | |
| | CON-Schim.6R.DS.SOSIP.664_OPT_N138X_N141X | | |
| HV1301405 | CON-Schim.6R.DS.SOSIP.664_OPT | V and | FIG. 10 |
| | | VI in | |
| | | FIG. 3A | |
| HV1301258_N301A | CON-Schim.6R.DS.SOSIP.664_N301A_avi | | FIG. 10 |
| HV1301258_N332A | CON-Schim.6R.DS.SOSIP.664_N332A_avi | | FIG. 10 |
| HV1300111_avi_N137A | CON-Sgp140CFI_avi_N137A | | FIG. 10 |
| HV1300111_avi_N141A | CON-Sgp140CFI_avi N141A | | FIG. 10 |
| HV1300111_avi_V1_4Q | CON-Sgp140CFI_avi_V1_4Q | | FIG. 10 |
| HV1301521_c_sorta | CON-Schim.6R.DS.SOSIP.664_OPT_N130D_N135K_N138S_N141S_C-SortaseA | | FIG. 10 |
| | CONS gp160 | | FIG. 16 |
| | CONS gp160_N138X_N141X | | |
| | CONS gp160 | | |
| | N130X_N135X_N138X_N141X | | |
| HV1301521 | (originally | | FIG. 14 |
| | HV1301405_N130D_N135K_N138S_N141S)CON- | | |
| | Schim.6R.DS.SOSIP.664_OPT_N130D_N135K_N138S_N141S | | |
| | >HV1301405_N138S_N141S;CON- | | FIG. 14 |
| | Schim.6R.DS.SOSIP.664_OPT_N138S_N141S | | |
| >HV1301405_D1305V1; | CON- | | FIG. 12 |
| | Schim.6R.DS.SOSIP.664_OPT_D1305V1 | | FIG. 11A |
| | (V1 loop from 10.19 isolate) | | |

Positions of mutations are HXB2 numbering and the positions which are modified in deglycosylated envelopes are bolded and underlined in ConS gp160 sequence in FIG. 16.

The invention contemplates any other design, e.g. stabilized trimer, of the sequences described here in. For non-limiting embodiments of additional stabilized trimers see WO2014/042669, WO/2017151801, WO/2017152146 and WO/2018161049, all of which are incorporated by reference in their entirety, and F14 and/or Vt8 designs.

F14/Vt8 designs mutations are listed below (HXB2 numbering) with a brief explanation for each. All were originally placed in BG505 SOSIP. They were then screened via BLI of small scale transfection supernatants. From the BLI data F14, F15 and Vt8 were expressed, purified, and screened for CD4 binding and triggering.

These sets of mutations were then put into CH848 10.17 DT and CH505 M5 SOSIP (F14, Vt8, and F14+Vt8) in addition to a BG505 SOSIP F14+Vt8.

Alternative embodiments immediately follow the full sets and V3 locks below.

Full Set→Pack the BMS-626529 binding site and lock the layers in place

Set of mutations referred to as F1: V681, S115V, A204L, V208L, V255W, N377L, M426W, M434W, H66S.

Eliminate* N377L, M426W, and M434W ->Avoid over-packing the area - N377 may be important for folding (is not totally buried). Eliminate means, that F2 construct includes all F1 mutations except N337L, M426W, and M434W.

Set of mutations referred to as F2: V68I, S115V, A204L, V208L, V255W, H66S

Eliminate S115V→Adding a V may be too large for the area

Set of mutations referred to as F3: V68I, A204V, V208L, V255L, H66S

Eliminate A204V→Adding a V may be too large for the packed region A204 resides (adding E causes opening of the apex)

Set of mutations referred to as F4: V68I, S115V, V208L, V255L, H66S

Retain N377L for minimal set→Above tested N377L elimination from full set, test here whether N377L stabilizes

Set of mutations referred to as F5: V68I, S115V, A204L, V208L, V255W, N377L, H66S

Add W69L to minimal set→previous work suggests aromatic residues in position 69 are destabilizing—test here

Set of mutations referred to as F6: V68I, S115V, A204L, V208L, V255L, W69L

Use W69V instead of W69L→test whether side chain length alters potential stabilizing effect

Set of mutations referred to as F7: V68I, S115V, A204L, V208L, V255L, W69V

Use W69A instead of W69L/V→further test whether side chain length alters potential stabilizing effect

Set of mutations referred to as F8: V68I, S115V, A204L, V255L, V208L, W69A

Reintroduce M426W→test a minimally reduced set—effect of M's

Set of mutations referred to as F9: V68I, S115V, A204L, V208L, V255W, N377L, M426W, H66S

Reintroduce M434W→test a minimally reduced set—effect of M's

Set of mutations referred to as F10: V68I, S115V, A204L, V208L, V255W, N377L, M434W, H66S

Introduce additional H72 mutation→can P favor loop turn stabilizing TRP69 Loop in W bound state

Set of mutations referred to as F11: V68I, S115V, A204V, V208L, V255L, H72P, H66S

Test minimal set with H66K rather than S→is charge a better solution to polar switch

Set of mutations referred to as F12: V681, S115V, V208L, V255L, H66K

Eliminate H66S from F1→H66 may be important for loop configuration

Set of mutations referred to as F13: V681, S115V, A204L, V208L, V255W, N377L, M426W, M434W

Minimal Set 2→Eliminate H66 and swap S115V for A204V; H66 could be important for loop and A204 my better stabilize that S115V

Set of mutations referred to as F14: V681, A204V, V208L, V255L

Minimal Set 3→Add N377L to test for further stabilization

Set of mutations referred to as F15: V681, A204L, V208L, V255W, N377L

V3 lock—Full Set

Set of mutations referred to as Vt1: Y177F, T320L, D180A, Q422L, Y435F, Q203M, E381L, R298M, N302L, N300L

Eliminate R298M and E381L→Determine whether these two are stabilizing rather than destabilizing

Set of mutations referred to as Vt2: Y177F, T320L, D180A, Q422L, Y435, Q203M, N302L, N300L

Eliminate E381L→Determine whether this residue is required to stabilize R298

Set of mutations referred to as Vt3: Y177F, T320L, D180A, Q422L, Y435, Q203M, R298M, N302L, N300L

Eliminate R298M→Determine whether this reside stabilizes E381

Set of mutations referred to as Vt4: Y177F, T320L, D180A, Q422L, Y435, Q203M, E381L, N302L, N300L

Retain Y177 and Y435→May stabilize interior through H-bonding

Set of mutations referred to as Vt5: T320L, D180A, Q422L, Q203M, E381L, R298M, N302L, N300L

Retain Y177 and Y435 while eliminating R298 and E381 mutations→A minimal set avoiding possible problems from charged pair mutations

Set of mutations referred to as Vt6: T320L, D180A, Q422L, Q203M, N302L, N300L

Dennis Burton Set→Control for comparison

Set of mutations referred to as Vt7: R298A, N302F, R304V, A319Y, T320M

Eliminate D180A→D180 appears to be destabilizing but may be stabilizing

Set of mutations referred to as Vt8: T320M, Q422M, Q203M, N302L, N300L

Add S174V→S174 is on the periphery but may be stabilizing with a hydrophobe

Set of mutations referred to as Vt9: T320M, Q422M, Q203M, N302L, N300L, S174V

Set (DS-SOSIP.4mut)→Additional Control Set

Set of mutations referred to as Vt10: I201C, A443C, L154M, N300M, N302M, T320L

*In the above description, “eliminate” means that full set number “N” construct includes all full set number “N−1” mutations except the mutations identified as eliminated.

Contemplated also are subsets of the mutations within a set. In a non-limiting embodiment, the mutations in Set F14 could be further parsed out to determine if there are fewer mutations or combinations of fewer mutations than in Set 14 which provide stabilization of the trimer.

In certain embodiments the invention provides an envelope comprising 17aa V1 region without N133 and N138 glycosylation, and N301 and N332 glycosylation sites, and further comprising “GDIR” motif see Ex. 1, wherein the envelope binds to UCAs of V1V2 Abs and V3 Abs.

### EXAMPLES

The following specific examples are to be construed as merely illustrative, and not limitative of the remainder of the disclosure in any way whatsoever. Without further elaboration, it is believed that one skilled in the art can, based on the description herein, utilize the present invention to its fullest extent.

### Example 1

### Pan-bnAb-Engaging Immunogens

Example 1A: This example describes design of HIV-1 envelopes antigenic for cross-epitope bnAb UCAs.

The discovery of broadly neutralizing antibodies (bnAbs) in HIV-1 infected individuals has provided evidence that the human immune system can target highly conserved epitopes on HIV-1 envelope. However, bnAbs have not been reproducibly induced with a vaccine, in primates. One approach to improve the induction of bnAbs is to specifically design immunogens that bind to the precursor B cell that gives rise to the bnAb. While highly affinity matured HIV-1 bnAbs react with many Envelope proteins, their precursors bind only to select Envs. Currently, immunogens exist that can bind to a single bnAb precursor. These Envs have the disadvantage of relying on a single bnAb precursor to be present in most individuals. If the bnAb precursor antibody is not present in that individual then the vaccine will not have the intended effect of inducing a specific type of antibody response. To improve the chances that an individual has the bnAb precursor that can engage the vaccine immunogen, we created a vaccine immunogen that can bind to multiple bnAb precursors. We designed the immunogen to interact with bnAbs precursors that interact with the first and second variable loop and glycans proximal to this loop—an epitope called V1V2-glycan. Secondly, the immunogen was also designed to interact with a bnAb precursor that bound to the third variable region and surrounding glycans on HIV-1 envelope—the V3-glycan site.

The immunogen was designed by creating a chimera of two HIV-1 envelope sequences that were derived from the HIV-1 infected individual CH0848 (See WO/2017152146 and WO/2018161049 references). The first Env CH0848.3.D0949.10.17 is antigenic for V3-glycan antibodies and was selected because it had a short first variable region in Env and bound to a V3-glycan antibody that possessed only 5 mutations (Bonsignori et al STM 2017). We modified this Env by removing glycosylation sites at 133 and 138 and found V3-glycan antibodies bound better to the Env when the glycosylation site was removed. These two glycosylation sites were identified as inhibitory in a neutralization screen where glycosylation sites on Env were removed to determine which glycans were required for neutralization by V3-glycan antibodies. For the CH0848.3.D0949.10.17 envelope we removed the glycosylation by substituting asparagine for amino acids that normally occur at positions 133 and 138 in other viruses. This glycan-modified Env bound with low nanomolar affinity to the V3-glycan bnAb precursor DH270 UCA3. To determine if a similar Env may have been present in the infected individual and could have potentially initiated the V3-glycan lineage in vivo, we screened all of the autologous virus sequences isolated from the infected individual CH0848 for viruses with a 17 amino acid variable region 1 and no glycans within the variable region except at position 156. We identified two sequences, with these characteristics. The first sequence CH0848.3.D1305.10.19 was produced as a recombinant protein. In biolayer interferometry assays it did not bind to V3-glycan antibodies. We created a pseudovirus expressing this Env and also found that V3 glycan antibodies did not neutralize it. However, we found that V1V2-glycan antibodies could bind to the recombinant protein. This was in contrast to CH0848.3.D0949.10.17, which lacked binding to V1V2-glycan bnAbs and precursors but was antigenic for V3-glycan antibodies. We inspected the sequences of the V1V2 and V3 regions and found that CH0848.3.D1305.10.19 lacked three glycans at positions 295, 301, and 332 usually bound by V3-glycan antibodies. To restore these V3 proximal glycosylation sites in CH0848.3.D1305.10.19 we used the V3 sequence of CH0848.3.D0949.10.17—the new envelope referenced as 19CV3. The modification of the CH0848.3.D1305.10.19 sequence to 19CV3 resulted in the addition of glycosylation sites at positions 301 and 332. We again made a recombinant protein of the chimeric envelope and found it bound to V1V2-glycan bnAbs as well as V3-glycan bnAbs—a combination of the phenotypes of the two parental envelopes. We next tested the binding of the bnAb precursors for V1V2 and V3-glycan sites. We found that 19CV3 bout to the bnAb precursor for two V1V2 glycan bnAb, CHO1 and VRC26, and V3 glycan Ab DH270.

With reference to CH0848 10.17DT SOSIP sequence, see W02018/161049, incorporated by reference in its entirety.

For non-limiting examples of hole-filled CH848 703010848.3.d0949.10.17 envelopes see WO/2017152146 and WO2018/161049, inter alia without limitation, FIGS. 44A-D, incorporated by reference in their entirety.

The immunogens of the invention can be delivered by any suitable mechanism.

In non-limiting embodiments, these could be Adeno-associated virus (AAV) vectors; Non-replicating viral vectors; vectors which provide sustained expression of the immunogen;

Vectors which can transduce dendritic cells, which present transgene(immunogen) in complex with MHCII to naive T cells. Constant antigen production could lead to improved clonal persistence, enhanced germinal center reactions, and higher somatic mutation;

In certain embodiments, the immunogens could be multimerized.

Example 1B—This example describes design of HIV-1 envelopes antigenic for cross-epitope bnAb UCAs--ConS envelope designs as panbnAb immunogens

To cover the diversity of HIV-1 isolates that circulate globally a consensus envelope was derived from all group M HIV-1 isolates available at the time (Liao HX et al Virology. 2006 Sep. 30; 353(2): 268-282., See also U.S. Pat. No. 8,071,107 and all parent applications and application claiming priority to) called CON-S. To induce neutralizing antibodies it is hypothesized that the immunogen should mimic the native, fusion-competent envelope on viruses. To create stable mimics of the HIV-1 Env CON-S we created SOSIP gp140s. The SOSIP gp140 was stabilized by introducing BG505 amino acids into the gp120 and gp41 regions as we have described previously (Saunders K O, Vercokzy L et al. Cell Reports. Volume 21, ISSUE 13, P3681-3690, Dec. 26, 2017). The Env was further stabilized by introducing a disulfide bond between amino acids at position 201 and 433 (Do-Kwon Y et al Nat Struct Mol Biol. 2015 July;22(7):522-31. doi: 10.1038/nsmb.3051. Epub 2015 Jun. 22.).

The CON-S sequence was furthered optimized to bind to antibodies that target the V3-glycan broadly neutralizing site by removing glycans that were determined in neutralization assays to inhibit V3-glycan antibody binding and neutralization. We hypothesize that broadly neutralizing antibody precursors have low affinity for HIV-1 Env which necessitates reducing steric barriers and glycosylation changes that hinder precursor antibody binding. In neutralization assays we identified that glycans attached between N131 and N141 prohibited neutralization by precursor antibodies that were developing neutralization breadth.

To improve binding to the V3-glycan site on CON-S stabilized gp140 SOSIPs we removed glycosylation sites at 130, 135, 138, and 141 by substituting asparagine for naturally occurring amino acids identified in the HIV-1 sequence database. The mutant Env contained N130D, N135K, N138S, and N141S mutations. Using mass spectrometry we verified that the glycans at 295, 301, and 332 were still the high mannose glycans preferentially bound by broadly neutralizing antibodies PGT128, PGT124, PGT135, DH270, BF520, and BG18. While removal of the V1 glycans may allow better binding to Env, the affinity for Env may be low for certain V3-glycan bnAb precursors. It has been shown that B cell receptors recognize low affinity antigen better when it is presented on a surface rather than free in solution (Batista F and Neuberger M J EMBO 2000, 19(4):513-520). Thus, we took the Env and arrayed it on the surface of a ferritin nanoparticle so that 8 copies of the CON-S SOSIP trimer could be displayed to B cells to maximize avidity of the BCR: SOSIP interaction. In total, a stabilized soluble HIV-1 Env trimer was derived from a consensus of group M and inhibitory glycans were removed to promote V3-glycan bnAb precursor binding. The optimized Env was arrayed on ferritin nanoparticles to enhance avidity between Env and B cell receptors.

The removal of four glycans in the V1 loop was hypothesized to permit binding of Env to unmutated bnAb precursors to initiate bnAb lineages. To select the bnAb intermediate antibodies within a lineage that are acquiring the ability to bind to multiple native Envs, we created a CON-S SOSIP Env trimers that added back the N130 and N135 glycosylation sites. This Env lacks glycosylation sites at 138 and 141 functions to select the antibodies that bind to Env with the correct mode to accommodate the N130 and N135 glycans. In a sequential vaccine this Env would be administered after 4 glycan deleted Env but before the wildtype Env so that glycans are sequentially added back to the Env to select the small population of B cells that recognize the V3-glycan site with the correct binding orientation.

### Example 2

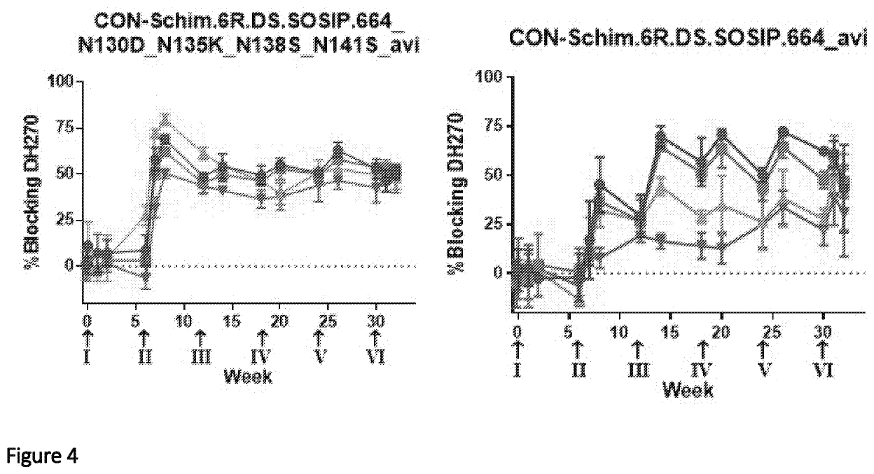

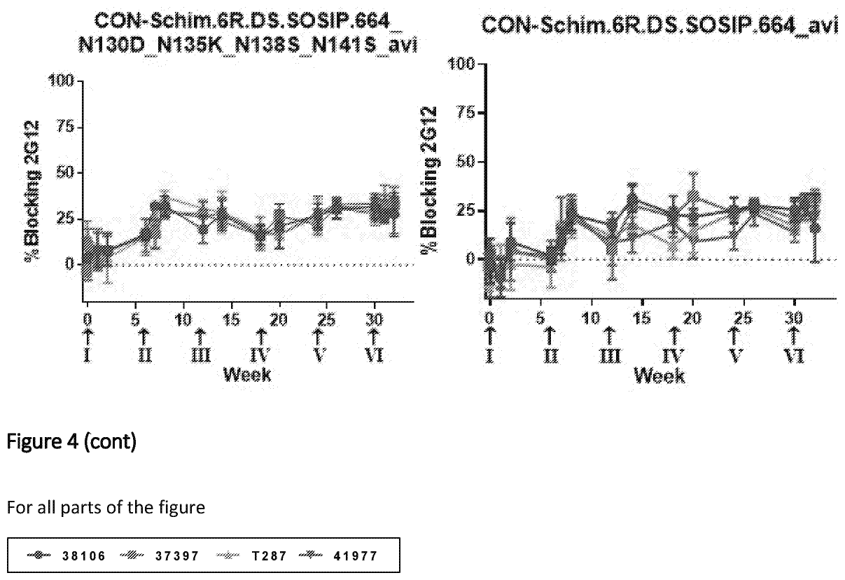

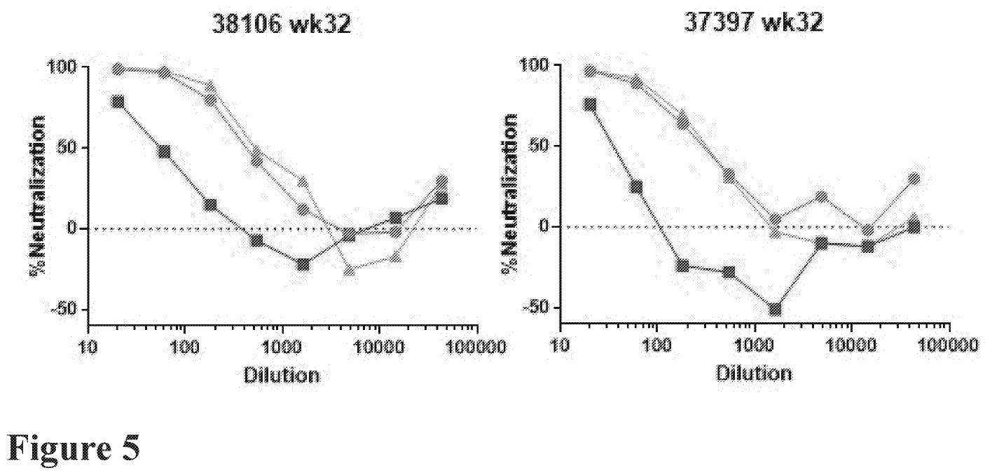

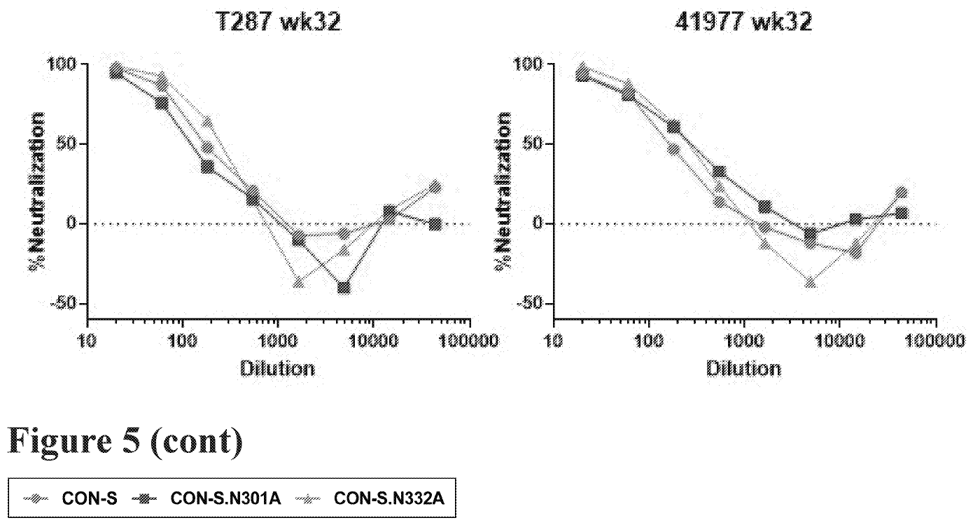

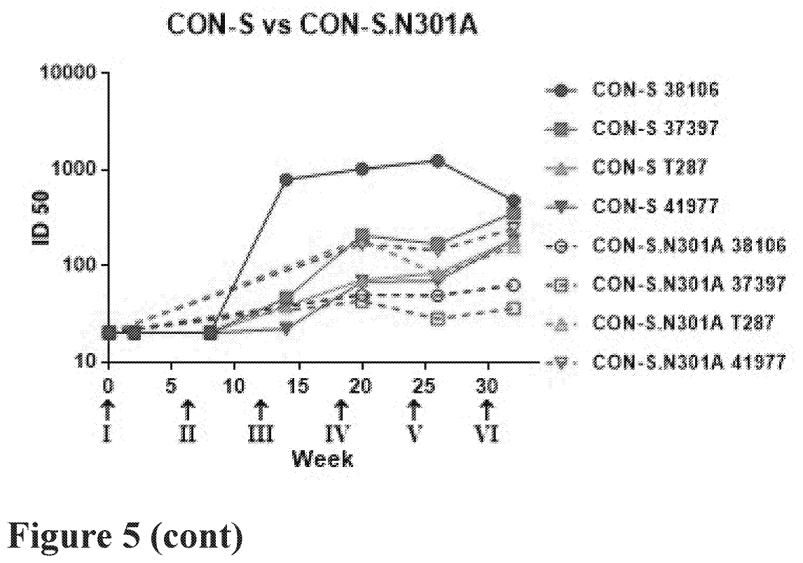

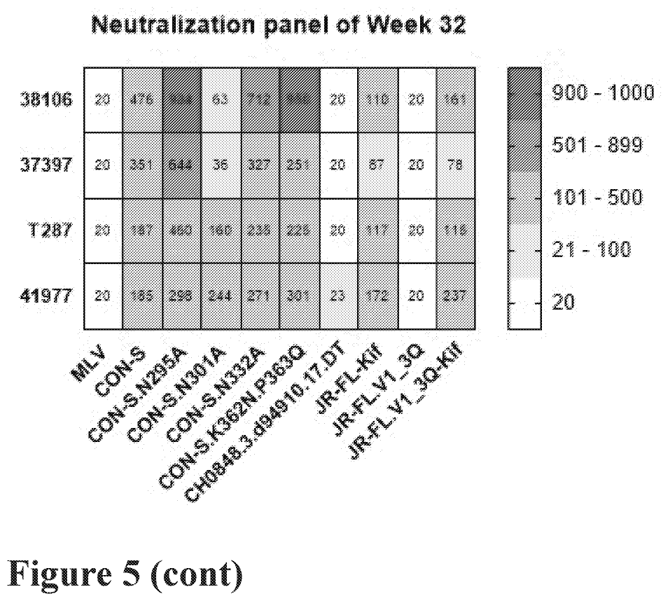

Glycan-optimized trimeric HIV-1 envelope elicits glycan-dependent autologous tier 2 neutralizing antibodies in rhesus macaques (See FIGS. 1-6, 18 et seq)

This example is based on the hypothesis that: Nanoparticle immunogens are necessary to overcome the low affinity between V3-glycan bnAb precursors and HIV-1 Env; HIV-1 Env should be enriched for Man9GlcNAc2 in order to optimally engage V3-glycan bnAb precursors; V1 glycans are inhibitory for early intermediate antibodies, thus sequential selection of antibodies that can accommodate V1 glycans will be necessary.

Introduction: Vaccine elicitation of broadly neutralizing antibodies (bnAbs) against HIV-1 has yet to be achieved. The target of bnAbs is HIV-1 envelope (Env) which is shielded by host glycans that hinder its recognition by antibodies. During natural infection, bnAbs develop that recognize the glycans and peptide proximal to the third variable region (V3-glycan). These glycan-dependent antibodies are protective in nonhuman primate models of HIV-1 infection. We previously observed that reactivity with Env was enhanced for V3-glycan bnAbs when the Env was enriched for Man9GlcNAc2 glycans or when V1 glycans were removed. We hypothesize that glycan-dependent bnAbs can be induced in primates with a vaccine if the immunogens are optimized to engage V3-glycan bnAb precursors and subsequently select for B cells within those lineages that are developing neutralization breadth.

The scientific premises of the non-human primate study (NHP145) is V3 glycan precursors prefer kif treated Env; A multimer is needed to activate the germline precursors because the affinity is so low; V3 glycan precursors have to learn to accommodate processed glycans one at a time

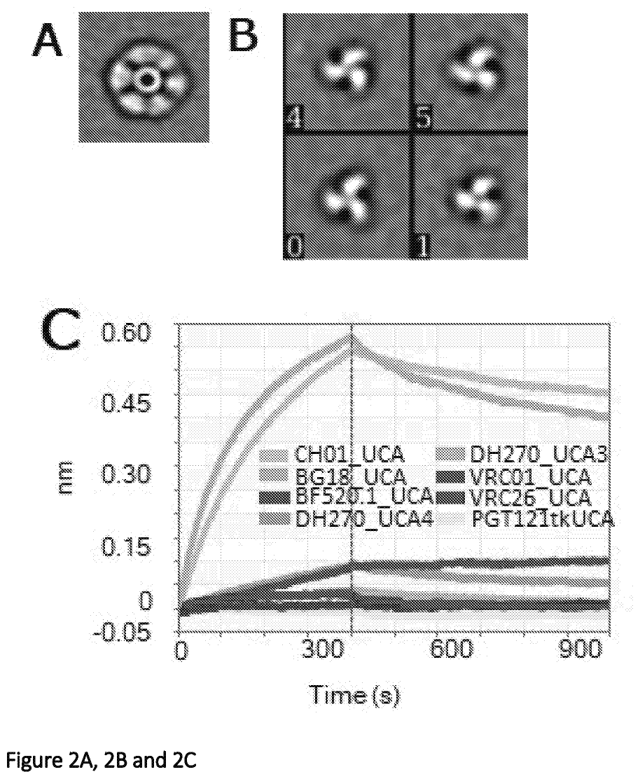

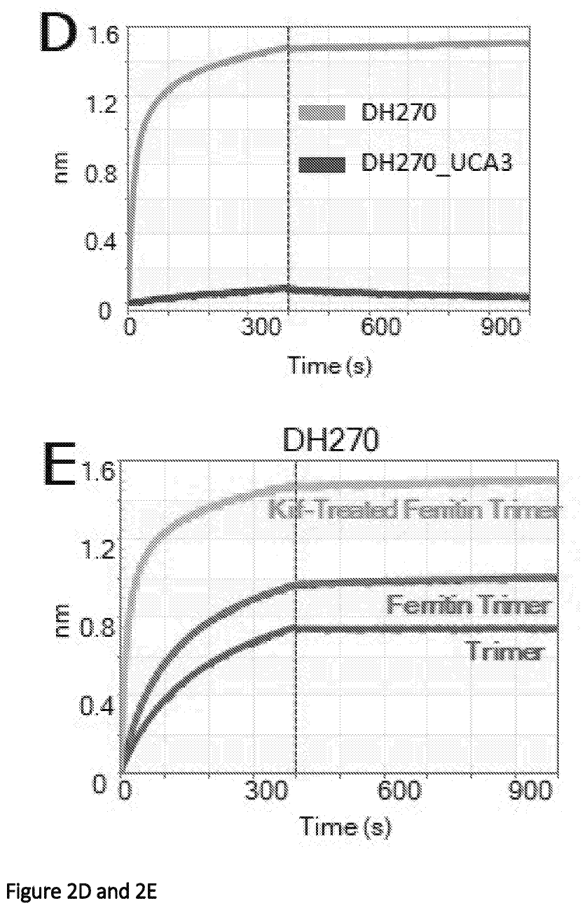

Methods: Recombinant trimeric HIV-1 CON-S Env was made as a SOSIP trimer and arrayed on ferritin nanoparticles. To enrich for Man9GlcNAc2 some Env were treated with kifunensine (kif). Trimer formation was determined by negative stain electron microscopy (EM). Antigenicity of the Envs was determined by Bio-layer interferometry. Four rhesus macaques were vaccinated 6 times with a series of HIV-1 Env glycosylation variants optimized to be antigenic for V3-glycan bnAbs as shown in FIG. 2. Binding and neutralizing antibodies were measured by ELISA and the TZM-bl assay respectively.

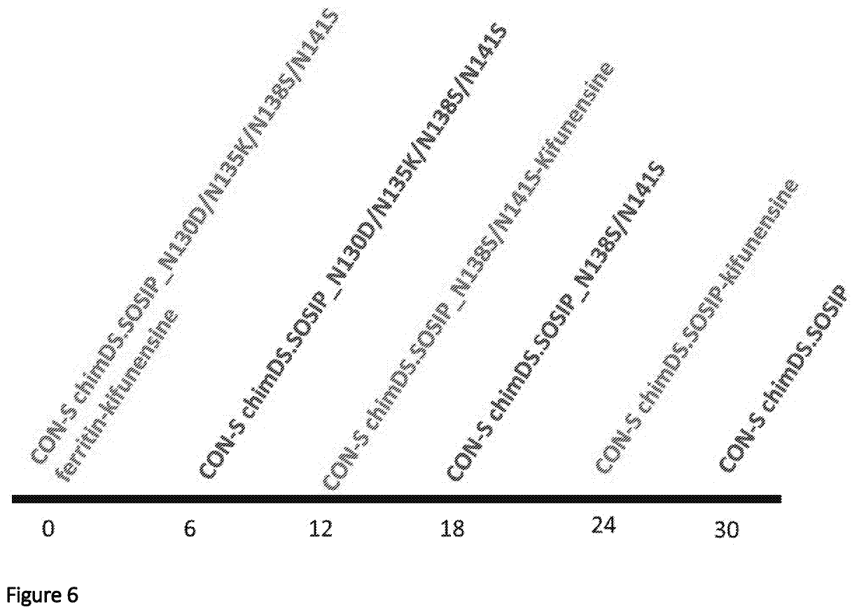

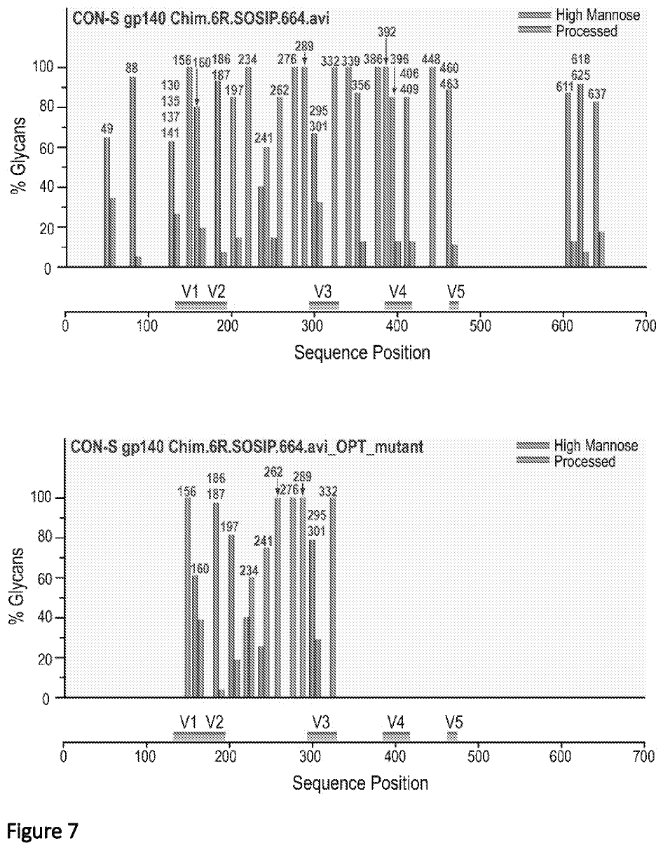

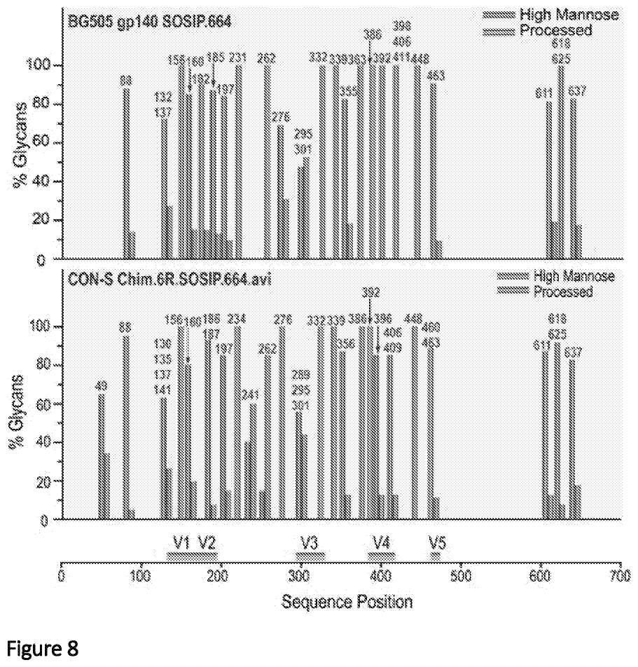

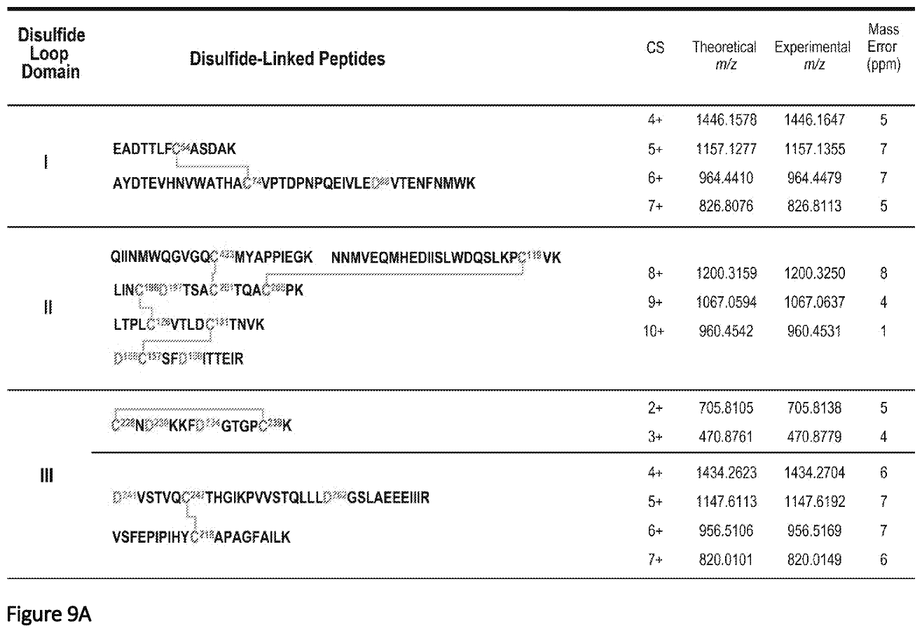

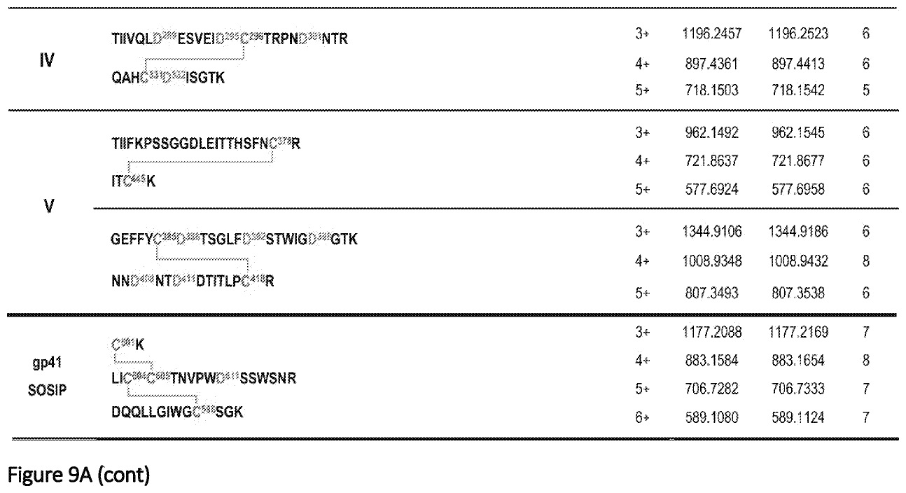

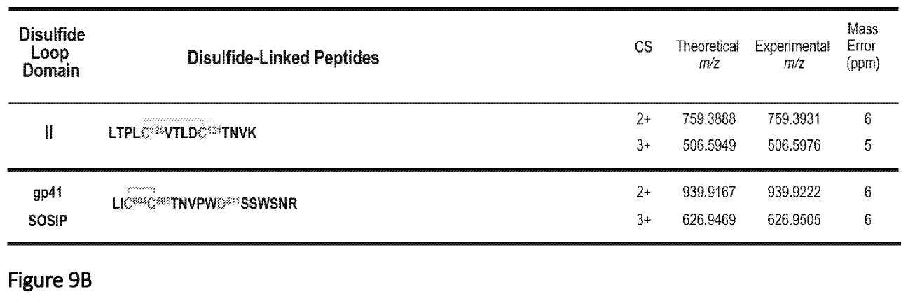

Animal study: Four non-human primates (NHPs) were immunized with the immunization regiment shown in FIG. 3A, FIG. 6. Recombinant protein was administered at a dose of 100 microgr in TLR4 adjuvant at every 6 weeks.

Conclusions:

Modified CON-S nanoparticles bind to the precursors of V3-glycan and V1V2 glycan bnAbs.

Multimerization of HIV-1 Env induces more durable antibody responses than free trimer.

Neutralizing antibody responses show that vaccination can elicit glycan-dependent neutralizing antibodies against the same Asn301 glycan targeted by bnAbs.

This example showed that CON-S SOSIP nanoparticle is antigenic for V3-glycan bnAb precursors. It also showed selection of sequential CON-S SOSIPs with glycan modifications boost glycan antibodies. The examples showed that this immunization regimen elicited autologous tier 2 neutralizing antibodies that did not target a glycan hole near N362. Autologous tier 2 neutralizing antibodies were N301 glycan-dependent in 2 of 4 macaques. The N301 glycan-dependent antibodies were distinct from DH501 in that they neutralized untreated CON-S, and thus did not require Man9GlcNAc2 enrichment.

References:

Stewart-Jones et al. Cell. 2016 May 5;165(4):813-26. doi: 10.1016/j.cell.2016.04.010. Epub 2016 Apr. 21.

Saunders et al., 2017 Cell Rep., 18 (2017), pp. 2175-2188.

Antibodies will be isolated (e.g. by single cell sorting), cloned and further analyzed for their properties including binding to autologous and heterologous envelopes, neutralization, etc. The goal is to determine types and specificities of induced antibodies, and whether any broad neutralizing or otherwise protective antibodies lineages are introduced.

Additional NHP studies could be conducted to determine whether immunization provides protective responses.

### Example 3

Con-S V1 delta glycans were also teste in Ca2+ flux assay.

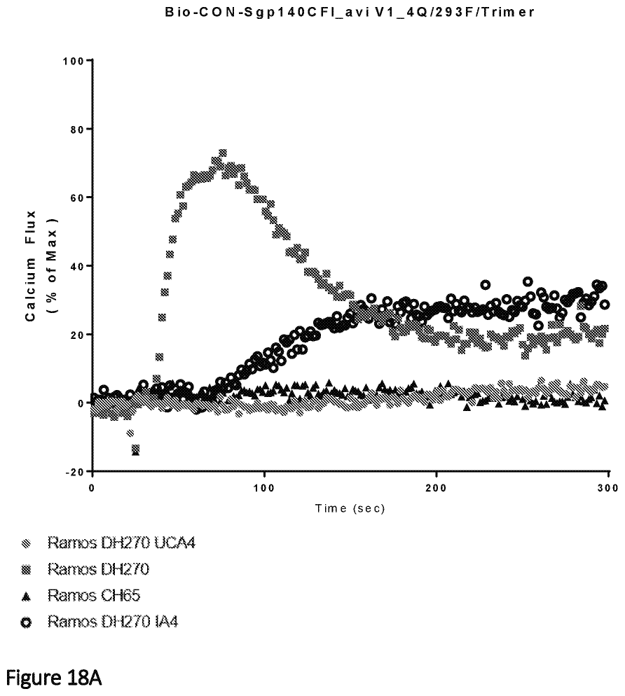

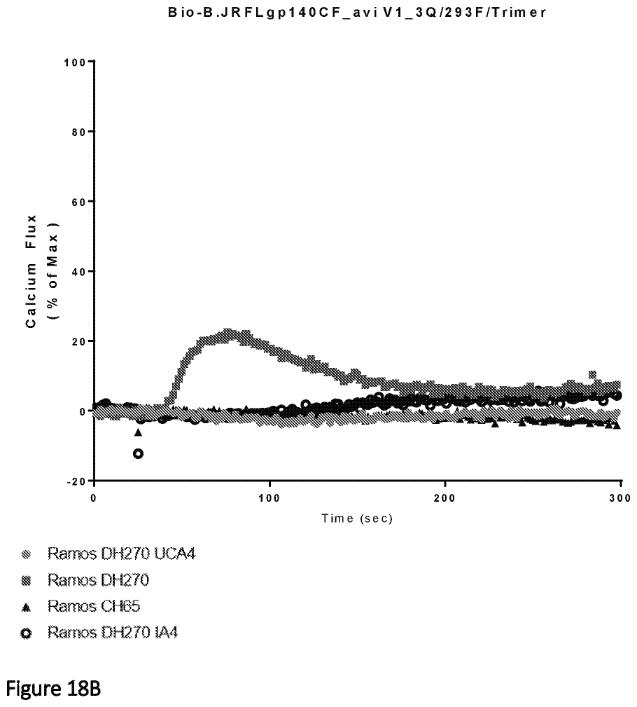

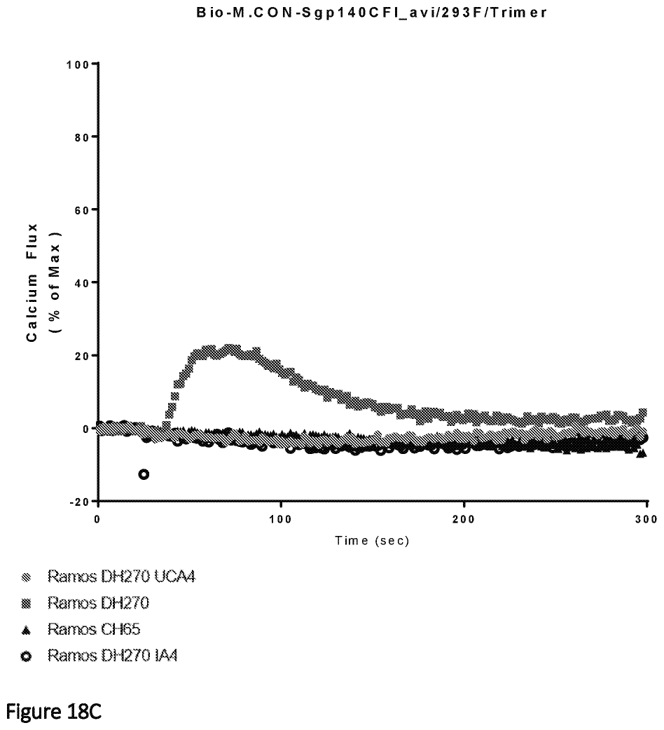

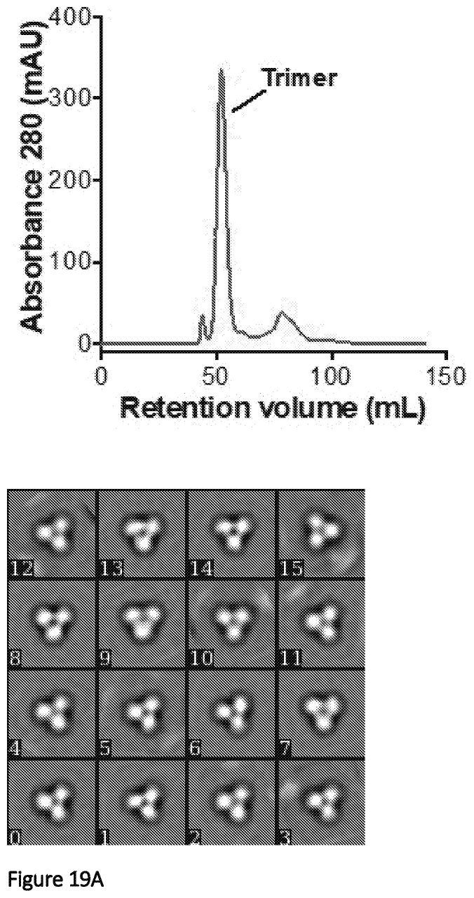

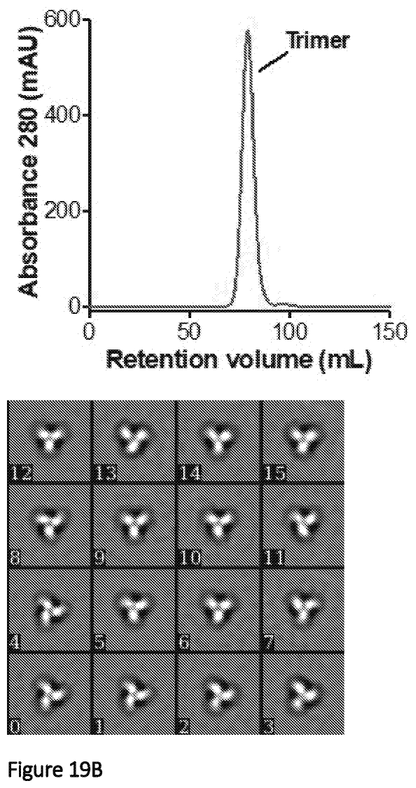

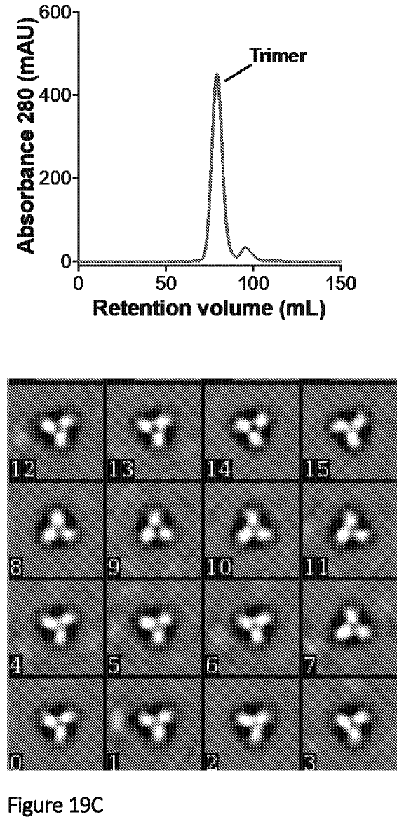

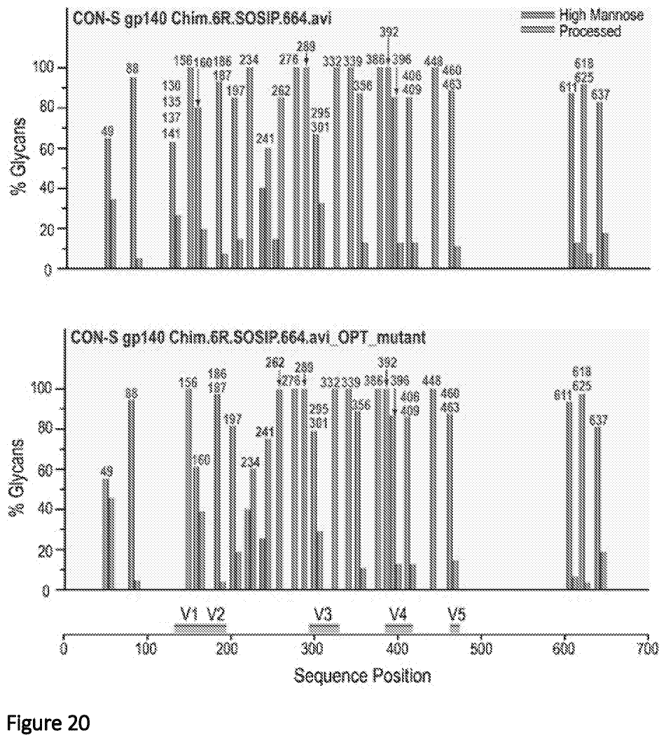

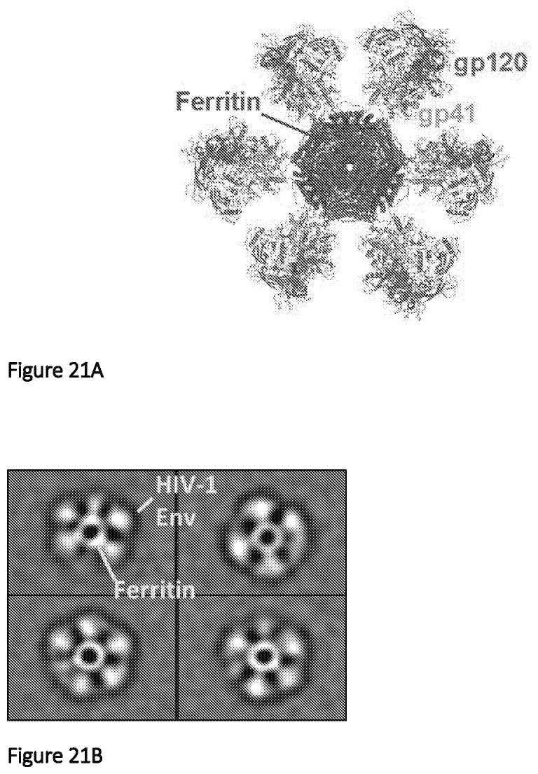

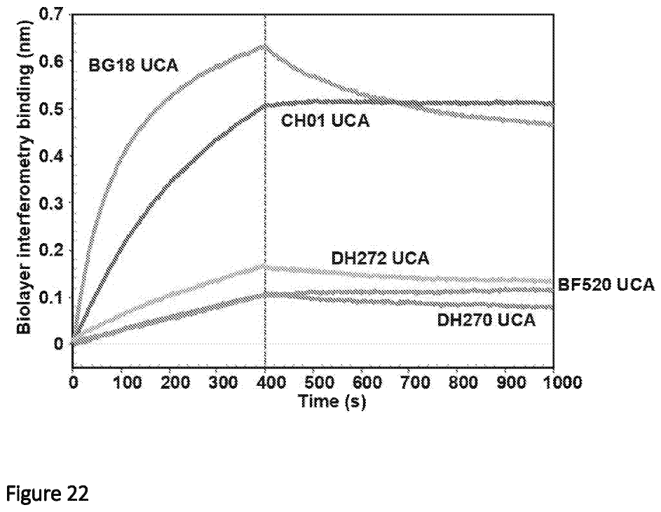

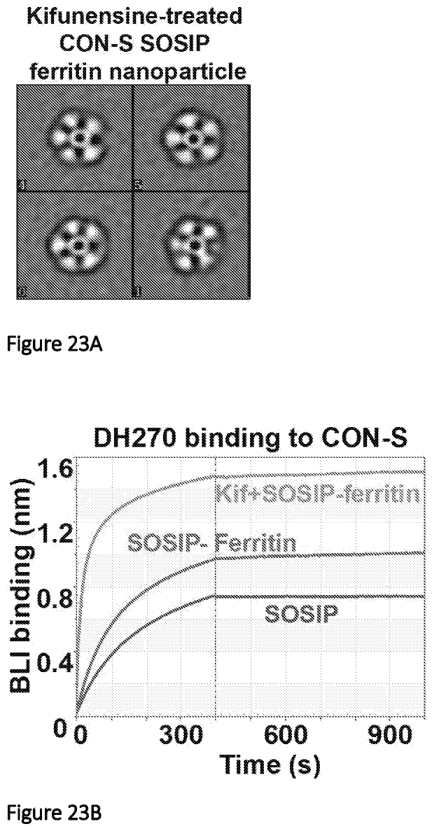

FIGS. 18A-18C show CON-S envelope induction of B cell receptor signaling in Ramos B cell lines expressing HIV-1 broadly neutralizing antibodies. The 3 antibodies are from three different points of maturation of the DH270 bnAb B cell lineage. In 18A the CON-S envelope inducing B cell receptor signaling in cells expressing the first intermediate antibody (DH270 IA4) from the DH270 lineage as well as a broadly neutralizing antibody (DH270) from the same lineage. They demonstrate that the envelope is antigenic for the earliest intermediate antibody within the DH270 lineage. In 18C, the presence of glycans in V1 of CON-S abrogates binding to DH270 IA4. The effect of glycan removal is not the same for another envelope JR-FL. The removal of glycans in V1 of JRFL is not sufficient to confer binding to the DH270 IA4 antibody.

Various recombinant proteins, trimers and/or nanoparticles were purified by chromatography, including antibody affinity chromatography (e.g. PGT145).

442EML(b) CON_Schim. 6R.DS SOSIP.664_OPT N130D_N135K_N138S_N141S_ferritin 5 uM-Kif 293F

455EML CONSchim.6R.DS.SOSIP.664_OPT_N130D_N135K_N138S_N141S_ferritin 293F

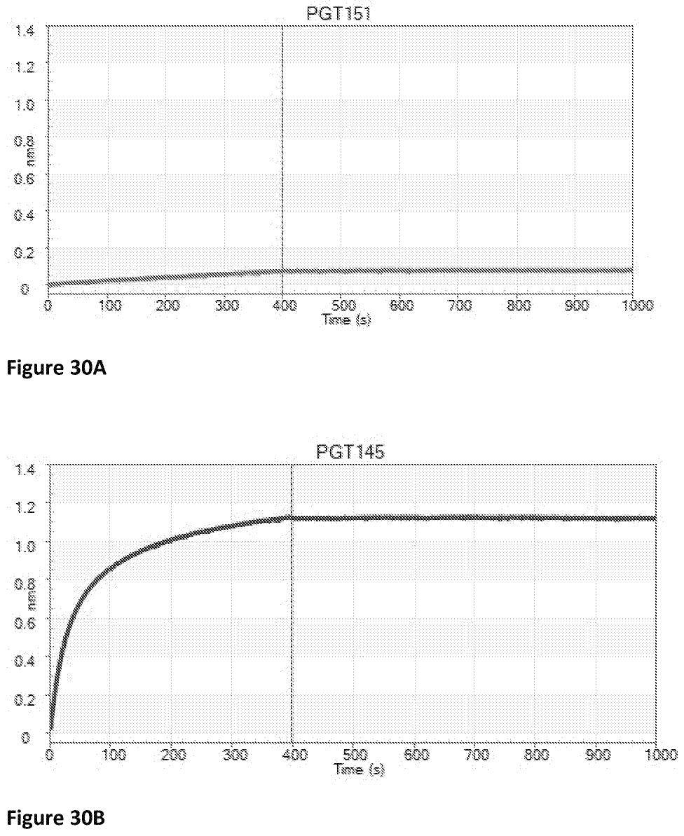

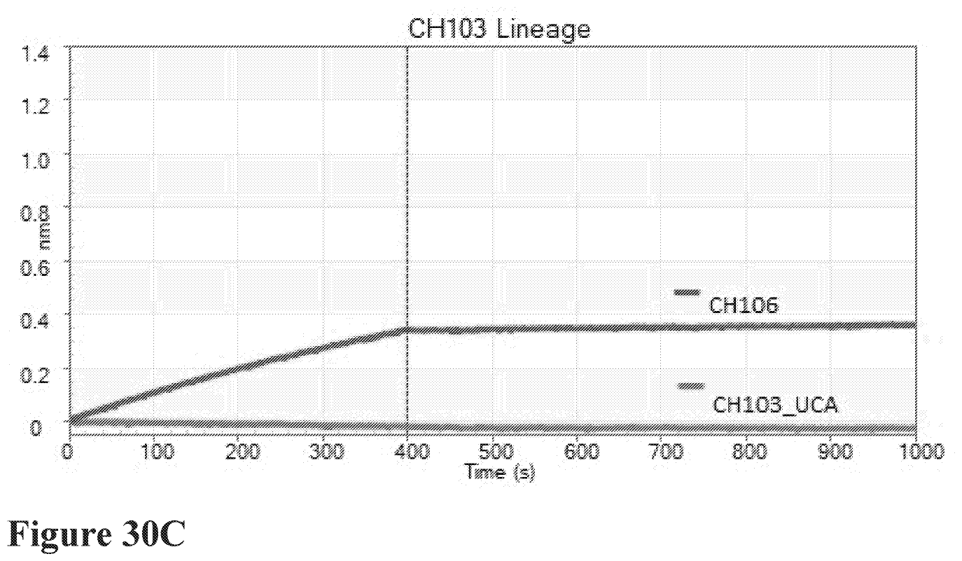

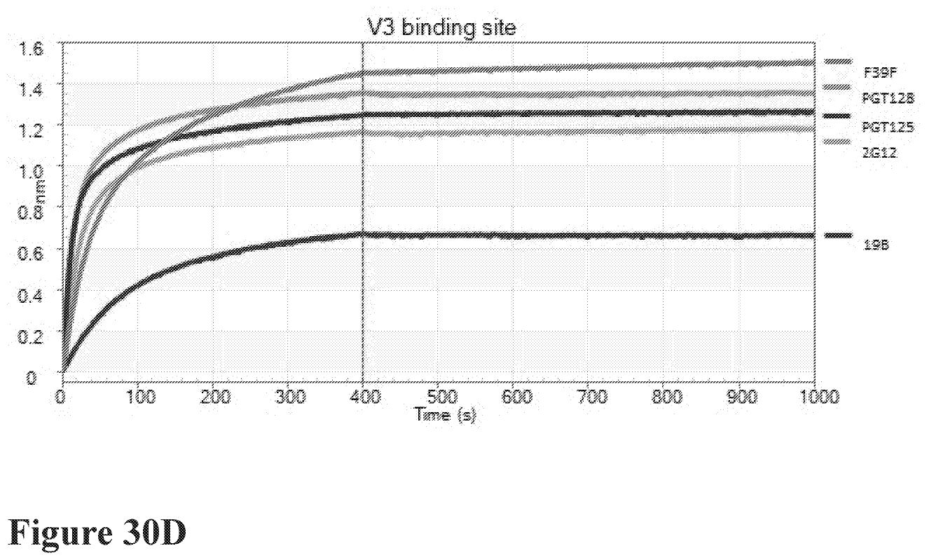

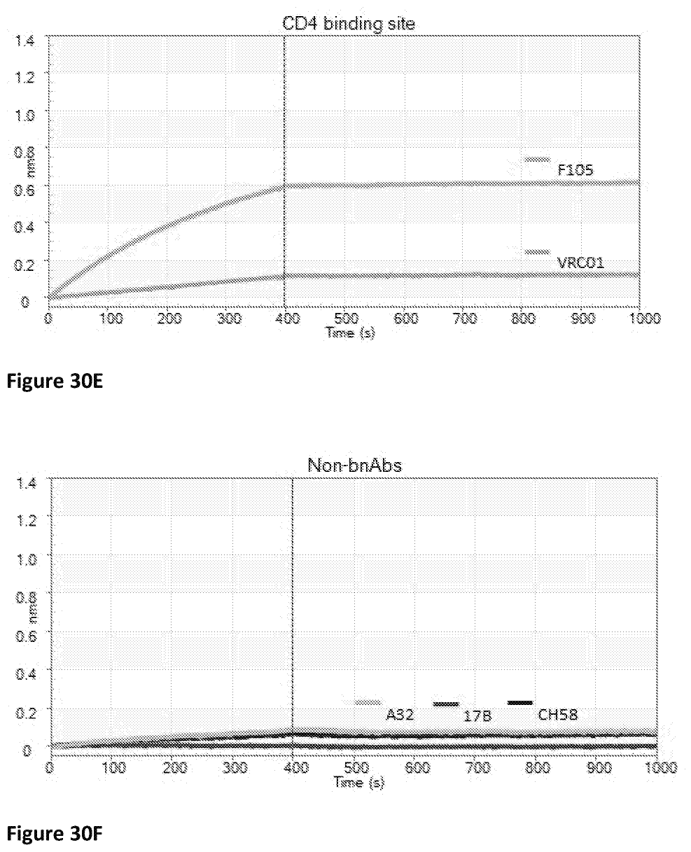

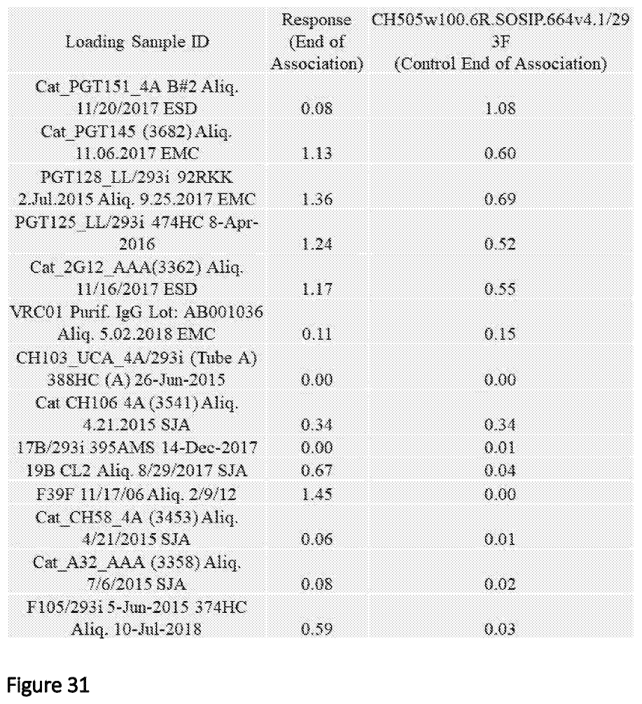

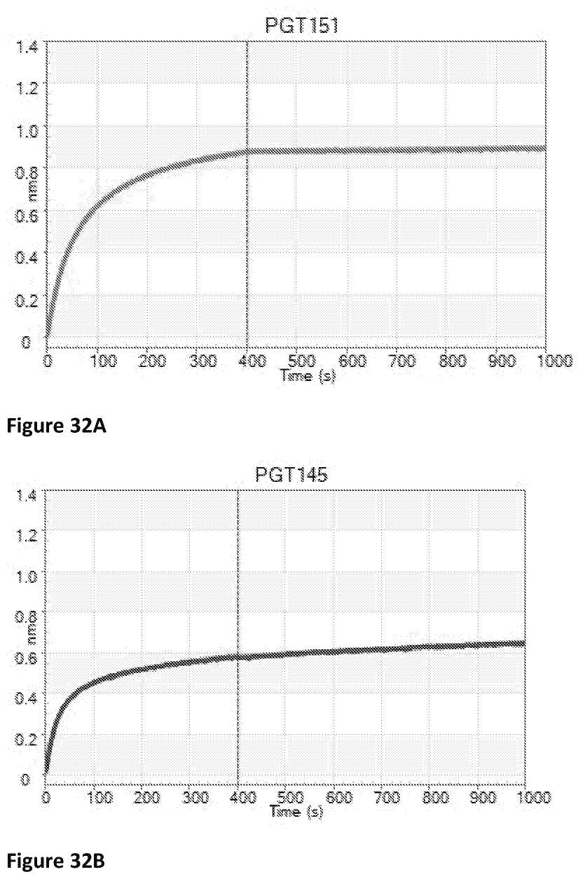

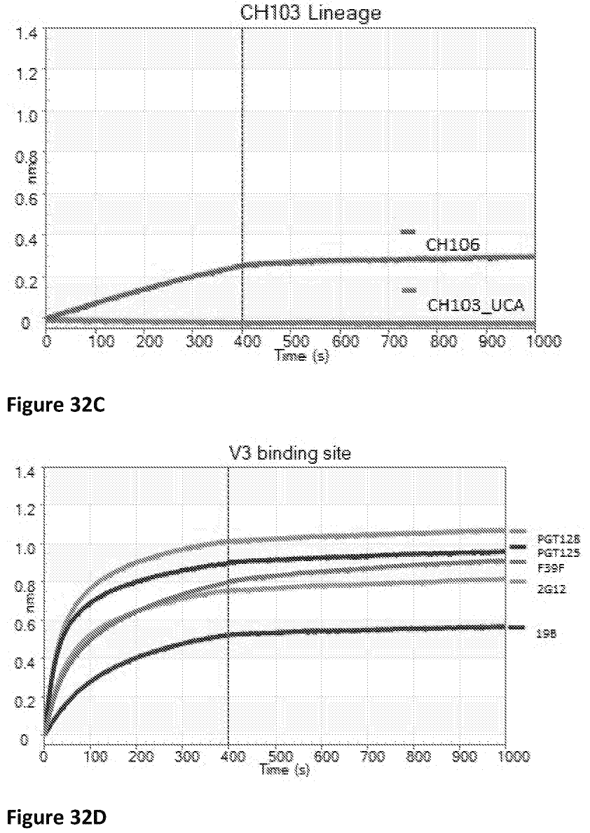

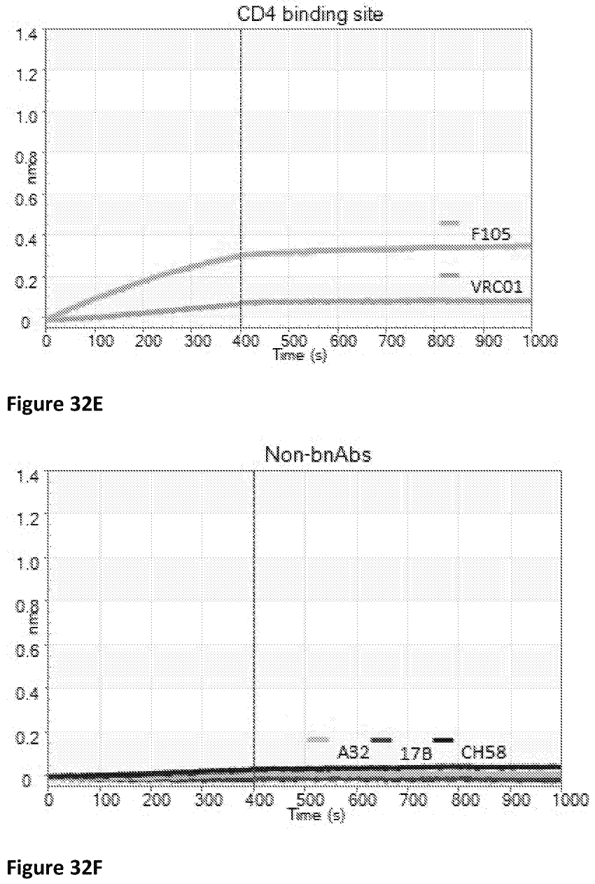

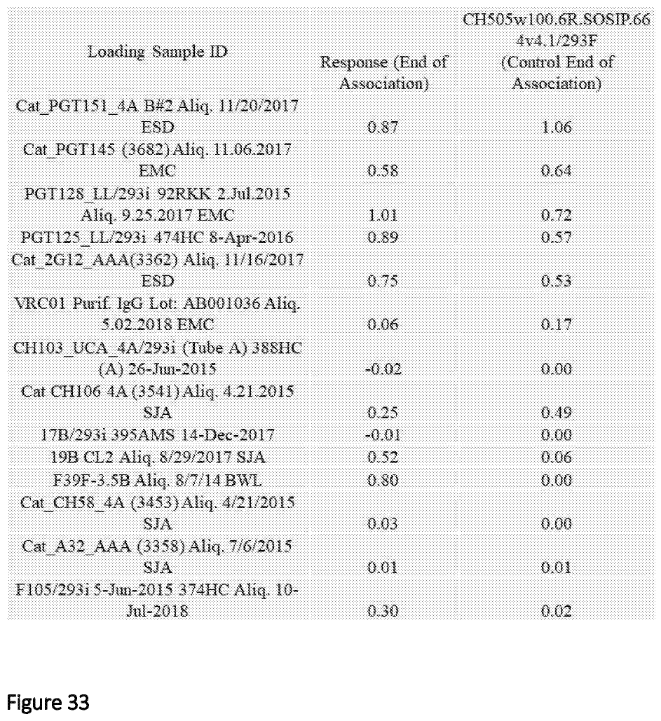

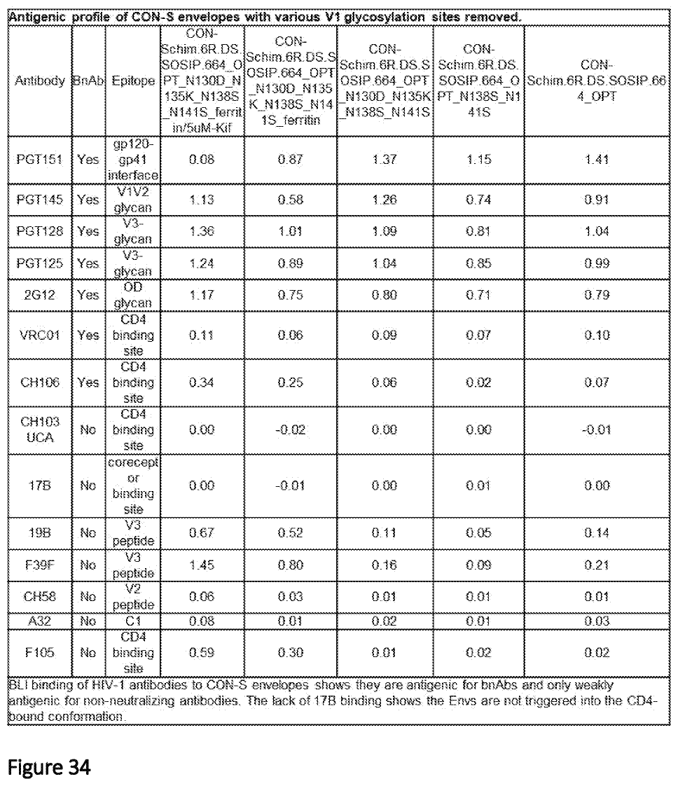

Antigenicity of recombinant ConS envelopes is shown in FIGS. 30-34.

What is claimed is:

1. A recombinant CON-S HIV-1 envelope comprising a V1 region, wherein the envelope lacks glycosylation at positions N138 and N141 (HXB2 numbering and FIG. 16).

2. A recombinant CON-S HIV-1 envelope comprising a V1 region, wherein the envelope lacks glycosylation at positions N130, N135, N138, and N141 (HXB2 numbering and FIG. 16).

3. A recombinant CON-S HIV-1 envelope comprising a 17aa-long V1 region, wherein the envelope lacks glycosylation at positions N138 and N141 (HXB2 numbering).

4. A recombinant CON-S HIV-1 envelope comprising a 17aa-long V1 region, wherein the envelope lacks glycosylation at positions N130, N135, N138, and N141 (HXB2 numbering).

5. The recombinant envelope of claim 1, wherein the envelope comprises all consecutive amino acids after the signal peptide of CON-Schim.6R.DS.SOSIP.664_OPT_N138X_N141X (Table 3).

6. The recombinant envelope of claim 1, wherein the envelope comprises all consecutive amino acids after the signal peptide of CON-Schim.6R.DS.SOSIP.664_OPT_N138S_N141S (Table 3, FIG. 10).

7. The recombinant envelope of claim 2, wherein the envelope comprises all consecutive amino acids after the signal peptide of CON-Schim.6R.DS.SOSIP.664 OPT N130X_N135X_N138X_N141X (Table 3).

8. The recombinant envelope of claim 2, wherein the envelope comprises all consecutive amino acids after the signal peptide of CON-Schim.6R.DS.SOSIP.664_OPT_N130D_N135K_N138S_N141S (Table 3, FIG. 10).

9. The recombinant envelope of claim 2, wherein the envelope comprises all consecutive amino acids after the signal peptide of CON-Schim. 6R.DS. SOSIP.664_OPT_N130X_N135X_N138X_N141X_ferritin (Table 3).

10. The recombinant envelope of claim 2, wherein the envelope comprises all consecutive amino acids after the signal peptide of CON-Schim.6R.DS.SOSIP.664_OPT_N130D_N135K_N138S_N141S_ferritin (Table 3, FIG. 10, FIG. 11B).

11. The recombinant envelope of claim 3, wherein the envelope comprises all consecutive amino acids after the signal peptide of CON-Schim.6R.DS.SOSIP.664_OPT_D1305V1 (Table 1, FIG. 13).

12. The recombinant CON-S envelope of any one of the preceding claims wherein the envelope is a protomer comprised in a stable trimer.

13. The recombinant envelope of any of claims 1-12, wherein the envelope is Man9GlcNAc-enriched.

14. A composition comprising the recombinant CON-S envelopes of any one of claims 1-13.

15. The composition of claim 14, wherein the composition comprises a carrier.

16. A nucleic acid encoding the recombinant envelopes of any one of claim 1-12.

17. A composition comprising the nucleic acid of claim 16 and a carrier.

18. The composition of claim 17, wherein the nucleic acid is a modified mRNA.

19. A composition comprising the recombinant CON-S envelope of any one of claim 1-13, wherein the recombinant CON-S envelope is multimerized and wherein optionally in some embodiments the envelope is comprised in a nanoparticle.

20. The composition of claim 19, wherein the recombinant CON-S envelope is comprised in a nanoparticle which is a ferritin nanoparticle.

21. A method of inducing an immune response in a subject comprising administering an immunogenic composition comprising the recombinant CON-S envelopes of any one of claim 1-13 or the composition of any one of claim 14, 15, or 18.

22. The method of claim 21, wherein the immunogenic composition comprises a first immunogen comprising all consecutive amino acids after the signal peptide of CON-Schim.6R.DS.SOSIP.664_OPT_N130D_N135K_N138S_N141S_ferritin (Table 3, FIG. 10, FIG. 11B), and wherein the immunogen is optionally Man9GlcNAc-enriched, optionally multimerized in a nanoparticle, wherein the nanoparticles is a ferritin nanoparticle.

23. The method of claim 22, further comprising administering a second immunogenic composition comprising a second immunogen comprising all consecutive amino acids after the signal peptide of CON-Schim.6R.DS.SOSIP.664_OPT_N130D_N135K_N138S_N141S (Table 3, FIG. 10, FIG. 11B), and wherein the immunogen is optionally multimerized in a trimer.

24. The method of claim 23, further comprising administering a third immunogenic composition comprising a third immunogen comprising all consecutive amino acids after the signal peptide of CON-Schim.6R.DS.SOSIP.664_OPT_N138S_N141S (Table 3, FIG. 10), wherein the immunogen is optionally Man9GlcNAc-enriched and optionally multimerized in a trimer.

25. The method of claim 24, further comprising administering a fourth immunogenic composition comprising a fourth immunogen comprising all consecutive amino acids after the signal peptide of CON-Schim.6R.DS.SOSIP.664_OPT_N138S_N141S (Table 3, FIG. 10), wherein the immunogen is optionally multimerized in a trimer.

26. The method of claim 25, further comprising administering a fifth immunogenic composition comprising a fifth immunogen comprising all consecutive amino acids after the signal peptide of CON-Schim.6R.DS.SOSIP.664_OPT_ (Table 3, FIG. 10), wherein the immunogen is optionally Man9GlcNAc-enriched and optionally multimerized in a trimer.

27. The method of claim 26, further comprising administering a sixth immunogenic composition comprising a sixth immunogen comprising all consecutive amino acids after the signal peptide of CON-Schim.6R.DS.SOSIP.664_OPT_ (Table 3, FIG. 10), wherein the immunogen is optionally multimerized in a trimer.

28. The method any one of claims 21-27, further comprising administering an adjuvant.

29. The method any one of claims 21-27, wherein the composition is administered as a prime and/or a boost, and optionally wherein the composition comprises nanoparticles.
